(19) United States
(12) Patent Application Publication
(10) Pub. No.: US 2018/0365372 A1
(43) Pub. Date: Dec. 20, 2018

(54) Systems and Methods for the Interpretation of Genetic and Genomic Variants via an Integrated Computational and Experimental Deep Mutational Learning Framework

(71) Applicant: Jungla Inc.

(72) Inventors: Carlos L. Araya, Palo Alto, CA (US); Jason A. Reuter, Palo Alto, CA (US); Samskruthi Reddy Padigepati, Sunnyvale, CA (US); Alexandre Colavin, Menlo Park, CA (US)

(21) Appl. No.: 16/011,753

(22) Filed: Jun. 19, 2018

Related U.S. Application Data
(60) Provisional application No. 62/640,432, filed on Mar. 8, 2018.
(60) Provisional application No. 62/521,759, filed on Jun. 19, 2017.

(51) Int. Cl. G06F 19/12; G06F 19/18; G06F 19/24

(52) U.S. Cl. CPC G06F 19/12; G06F 19/24; G06F 19/18

(57) ABSTRACT

Disclosed herein are system, method, and computer program product embodiments for determining phenotypic impacts of molecular variants identified within a biological sample. Embodiments include receiving molecular variants associated with functional elements within a model system. The embodiments then determine molecular scores associated with the model system. The embodiments then determine molecular signals and population signals associated with the molecular variants based on the molecular scores. The embodiments then determine functional scores for the molecular variants based on statistical learning. The embodiments then derive evidence scores of the molecular variants based on the functional scores. The embodiments then determine phenotypic impacts of the molecular variants based on the functional scores or evidence scores.

### CROSS REFERENCE TO RELATED APPLICATIONS

This application claims priority to U.S. Provisional Patent Application No. 62/521,759, filed on Jun. 19, 2017, now pending, and U.S. Provisional Patent Application No. 62/640,432, filed on Mar. 8, 2018, now pending, both of which are herein incorporated by reference in their entireties.

### OVERVIEW

Understanding the impact of genotypic (e.g., sequence) variants within functional elements in the genome—such as protein coding genes, non-coding genes, and regulatory elements—is critical to a diverse array of life sciences applications. Today, nearly half of all disease-associated genes harbor a higher number of uncharacterized variants in the general population than variants of known clinical significance. This poses significant challenges for both diagnostic and screening tests evaluating genetic and genomic sequences (Landrum et al. 2015; Lek et al. 2016). A high number of novel variants of unknown clinical significance is a feature of nearly all genes (e.g., for both germline and somatic variants in the population) and affects even the most frequently tested genes. For example, tests that evaluate gene-panels for cancer predisposing mutations report finding as many as 95 uncharacterized variants per known disease-causing variant (Maxwell et al. 2016). As such, predicting the phenotypic (e.g., cellular, organismal, clinical, or otherwise) consequences of genotypic variants is a hurdle to leveraging genetic and genomic information in a wide array of clinical settings.

Genotypic (e.g., sequence) variants within genomically-encoded functional elements can affect diverse biophysical processes, altering distinct molecular functions within each element, and resulting in varied clinical and non-clinical phenotypes. For example, in an established tumor suppressor protein coding gene, phosphatase and tensin homolog (PTEN), genotypic variants affecting transcription (f.g. −903G>A, −975G>C, and −1026C>A), protein stability (f.g. C136R), phosphatase catalytic activity (f.g. C124S, H93R), and substrate recognition (f.g. G129E), have all been associated with Cowden Syndrome (CS), presenting high-risks of breast, thyroid, endometrial, kidney, colorectal cancers and melanoma (Heikkinen et al. 2011; He et al. 2013; Myers et al. 1997; Myers et al. 1998). Variants affecting the same biophysical processes and molecular functions can lead to co-morbidities between distinct disorders, as exemplified by PTEN variants affecting phosphatase activity (e.g., H93R) which have been additionally implicated in autism spectrum disorder (ASD) (Johnston and Raines 2015), leading to frequent co-morbidities between ASD and cancers (Markkanen et al. 2016). Moreover, variants affecting distinct biophysical processes and molecular mechanisms within a functional element can present stereotypic, differentiated clinical and non-clinical phenotypes. Mutations in the lamina A/C gene (LMNA) cause a compendium of more than fifteen diseases collectively known as “laminopathies,” which include A-EDMD (autosomal Emery-Dreifuss muscular dystrophy), DCM (dilated cardiomyopathy), LGMD1B (limb-girdle muscular dystrophy 1B), L-CMD (LMNA-related congenital muscular dystrophy), FPLD2 (familial partial lipodystrophy 2), HGPS (Hutchinson-Gilford progeria syndrome), atypical WRN (Werner syndrome), MAD (mandibuloacral dysplasia) and CMT2B (Charcot-Marie-Tooth disorder type 2B) (Scharner et al. 2010). In LMNA, genotypic (e.g., sequence) variants leading to HGPS create a cryptic splice site donor in the lamin A-specific exon 11 that results in a truncated form of lamin A, whereas variants leading to FPLD2 alter surface charge of the Ig-like domain and do not change the crystal structure of the mutant protein (Scharner et al. 2010). Thus, disentangling the complexity of genotype-phenotype relationships across a wide array of variant types, functional elements, and molecular systems, and cellular effects is an outstanding challenge to robust, scalable interpretation of the phenotypic consequences of variants discovered in clinical and non-clinical genetic and genomic tests.

Indeed, assessment of the significance of genotypic (e.g., sequence) variants can be a complex and challenging task. As recently as 2015, a survey of variant classifications demonstrated that as many as 17% (e.g., 2,229/12,895) of variant classifications were inconsistent among classification submitters (Rehm et al. 2015). Between clinical testing laboratories, the concordance in interpretations has been measured to be as low as 34% though specific recommendations can increase inter-laboratory concordance to 71% (Amendola et al. 2016).

With greater than 5,300 genes evaluated by genetic tests (e.g., according to the NCBI Genetic Test Registry) in the market, scalable solutions for interpreting (e.g., classifying) genotypic (e.g., sequence) variants in a broad array of genes, diseases, and contexts (e.g., clinical and non-clinical) are critical to the efforts in the precision medicine and life sciences industries. With greater than 14,000,000 possible (e.g., unique) molecular variants within the subset of molecular variants corresponding to single nucleotide variants (SNVs), within the subset of coding sequences, and within the subset of protein-coding genes in the clinical testing market, effective solutions for molecular variant classification need to be robust and scalable.

While multiple strategies exist for identifying the phenotypic impacts of molecular variants—including but not limited to family segregation, functional assays, and case-control studies—at present, only computational variant impact predictors are able to provide supporting evidence at the required scale. In effect, an analysis of clinical variant classifications from practitioners following the joint guidelines for clinical variant interpretation from the American College of Medical Genetics and Genomics (ACMG) and the Association of Molecular Pathology (AMP) demonstrate that ˜50% of clinical variant classifications rely on the use of computational variant impact predictors. Yet, despite their wide use, benchmarking studies indicate that computational variant impact prediction algorithms—such as SIFT, PolyPhen (v2), GERP++, Condel, CADD, REVEL, and others—have demonstrably low performances, with accuracies (AUC) in the 0.52-0.75 range (Mahmood et al. 2017).

Direct assays of molecular function may provide a basis for the accurate interpretation of the clinical and non-clinical impacts of genotypic (e.g., sequence) variants (Shendure and Fields 2016; Araya and Fowler 2011). To date, a diverse spectrum of assays have been devised to directly assess the impact of variants on a wide array of molecular functions. However, existing methods require a priori knowledge or assumptions of the mechanism of action of variants associated with the clinical (and non-clinical) phenotypes under investigation to define the molecular functions to assay (Shendure and Fields 2016). These methods are often limited to capturing the effects of, and informing on, only variants affecting specific molecular functions assayed, imposing limitations on the types of variants, types of molecular functions, and types of functional elements and genes which can be assayed in large-scale. Thus, while a phosphatase assay, for example, can nominate (e.g., rule-in) potential disease-associations for variants affecting catalytic activity of the PTEN tumor suppressor, such assay may not be able to exclude (e.g., rule-out) potential disease-associations for variants affecting protein stability as these variants may increase risk of developing disease without observable defects in catalytic activity. Conversely, while a protein stability assay, for example, can nominate (e.g., rule-in) potential disease-associations for variants leading to stability defects in the PTEN tumor suppressor, such assay may not be able to exclude (e.g., rule-out) potential disease-associations for variants affecting catalytic activity. The potential need for a priori knowledge or assumptions of the mechanism of action (and hence relevant molecular functions to assay) may limit the application of these methods to well-characterized functional elements (e.g., genes) and phenotypes which may prevent their application to poorly understood disease-associated genes.

Building on the technological foundations of high-throughput DNA sequencing platforms, recently developed large-scale functional assays—such as Deep Mutational Scanning (DMS), HITS-KIN, RNA-MAP, and others—have enabled comprehensive or near-comprehensive coverage of the possible sequence variants of distinct sequence classes, including single-nucleotide variants (SNVs) and non-synonymous variants (NSVs, missense variants) in coding, non-coding, and regulatory elements (Fowler et al. 2010; Araya et al. 2012; Guenther et al. 2013; Buenrostro et al. 2014; Kelsic et al. 2016; Patwardhan et al. 2009). Such methods may serve as the basis for robust, statistically-validated interpretation of the impact of molecular variants—such as genotypic (e.g., sequence) variants—on patient phenotypes (Starita et al. 2015; Majithia et al. 2016), including clinical phenotypes such as lipodystrophy and increased risk of type 2 diabetes (T2D) in patients with variants in PPARG, or increased risk of breast and ovarian cancers in patients with variants in BRCA1. While such methods may provide robust variant interpretation in clinical and non-clinical testing settings, these methods may require significant development and customization to assay each molecular function and each functional element. This may limit their utility as a generalizable, scalable solution to systematically assess the clinical and non-clinical consequences of molecular variants—such as genotypic (e.g., sequence) variants—across diverse types of variants, biophysical processes, molecular functions, functional elements, genes, and ultimately, pathways. Thus, there is a need for a multi-functional platform and methods for variant impact assessment.

### BRIEF DESCRIPTION OF THE DRAWINGS

The accompanying drawings are incorporated herein and form a part of the specification.

FIGS. 1A-1C illustrate integrated functional assay and computational Deep Mutational Learning (DML) processes and systems for determining the phenotypic impact of molecular variants, as well as example (e.g., intermediate) data generated from the application of processes and systems in two genes of the RAS/MAPK family of disorders, according to some embodiments.

FIGS. 2A-2B illustrate the performance of Deep Mutational Learning (DML) processes and systems in the identification (e.g., binary classification) of disease-causing (e.g., pathogenic) and neutral (e.g., benign) molecular variants for germline (e.g., inherited) and somatic disorders in three genes of the RAS/MAPK pathway, HRAS, PTPN11, and MAP2K2, according to some embodiments.

FIGS. 3A-3B illustrate the performance of Deep Mutational Learning (DML) processes and systems in the identification (e.g., binary classification) of cells harboring germline disease-causing (e.g., pathogenic) or neutral (e.g., benign) molecular variants in MAP2K2, according to some embodiments.

FIG. 4 illustrates an architecture of a neural network-based Denoising Autoencoder trained and applied to generate robust, reduced representations of molecular scores, according to some embodiments.

FIG. 5 illustrates normalized ERK pathway activation measured as the fraction of total ERK protein phosphorylated through enzyme-linked immunosorbent assays of cellular extracts from H293 cells harboring control, wildtype, and mutant versions of MAP2K2 and PTPN11, according to some embodiments.

FIG. 6 illustrates an example of a method for reducing the costs of deploying Deep Mutational Learning (DML) to identify the phenotypic impact of molecular variants through the staged optimization and deployment of assays with varying cell-number, read-depth, Dimensionality Reduction Models (m_{DR}), and Functional Models (m_{F}), whereby optimization is first carried out on a (reduced) Truth Set of molecular variants, and deployment includes a Target Set of molecular variants, according to some embodiments.

FIG. 7 illustrates an example of a method for computing phenotype scores, according to some embodiments.

FIG. 8 illustrates an example of a method for computing molecular scores, according to some embodiments.

FIG. 9 illustrates methods for computing molecular signals associated with individual molecular variants, according to some embodiments.

FIG. 10 illustrates methods for computing molecular state-specific independent or disjoint estimates of molecular signals, according to some embodiments.

FIG. 11 illustrates methods for characterizing the distribution of cells with specific molecular variants across molecular states or phenotype scores, and deriving population signals, according to some embodiments.

FIG. 12 illustrates an example of a method for leveraging unsupervised learning techniques for identification of higher-order molecular signals from lower-order molecular signals associated with individual molecular variants, according to some embodiments.

FIG. 13 illustrates an example of a method for deriving functional scores and functional classifications via machine learning to associate molecular, phenotype, or population signals with phenotypic impacts of molecular variants via regression and classification techniques, according to some embodiments.

FIGS. 14A-14B illustrate an example of the performance of methods and systems for the binomial classification of molecular variants with two distinct phenotypic impacts as trained using varying numbers of cells, according to some embodiments.

FIG. 15 illustrates an example of a method that permits inferring sequence-function maps describing the functional scores or functional classifications for all possible non-synonymous variants in a protein coding gene using functional scores and functional classifications from a subset of the possible non-synonymous variants, according to some embodiments.

FIG. 16 illustrates an example of systems and methods for reducing the costs and increasing the scope of DML processes to determine the phenotypic impact of molecular variants through a series of modeling layers, according to some embodiments.

FIG. 17 illustrates an example of a method for generating lower-order Variant Interpretation Engines (VIEs) that can be gene and condition-specific using machine learning techniques, according to some embodiments.

FIG. 18 illustrates an example of a method for identification of Significantly Mutated Regions (SMRs) and Networks (SMNs), according to some embodiments.

FIG. 19 is an example computer system useful for implementing various embodiments.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, apparatus, device, method and/or computer program product embodiments, and/or combinations and sub-combinations thereof, for enabling multi-functional, multi-element, and multi-gene (e.g., pathway-scale) assessment of the phenotypic impact of variants across a wide array of variant types, biophysical processes, molecular functions, and phenotypes.

The present disclosure provides system, apparatus, device, method and/or computer program product embodiments that can leverage high-throughput molecular measurements (e.g., next-generation sequencing), single-cell manipulation, molecular biology, computational modeling, and statistical learning techniques and can enable multi-functional, multi-element, and multi-gene (pathway-scale) assessment of the phenotypic impact of variants across a wide array of variant types, biophysical processes, molecular functions, and phenotypes.=

The present disclosure provides system, apparatus, device, method and/or computer program product embodiments for systematically determining and statistically validating one or more phenotypic (e.g., clinical or non-clinical) impacts (e.g., pathogenicity, functionality, or relative effect) of molecular variants identified—such as genotypic (e.g., sequence) variants—in one or more (e.g., coding or non-coding) functional elements (e.g., protein-coding genes, non-coding genes, molecular domains such as protein or RNA domains, promoters, enhancers, silencers, regulatory binding sites, origins of replication, etc.) in the (e.g., nuclear, mitochondrial, etc.) genome(s), or their derivative molecules—within a biological sample or record thereof of a subject.

The present disclosure provides system, apparatus, device, method and/or computer program product embodiments for the classification (or regression) of likely phenotypic impacts in a subject on the basis of one or more molecular signals, phenotype signals, or population signals measured in in vivo or in vitro functional model systems. The derived regressions or classifications can be referred to as functional scores or functional classifications.

Embodiments herein represent a departure from existing computational or functional evidence support systems for molecular variant classification, as for example utilized in clinical genetic and genomic diagnostics.

First, while existing computational methods and systems for variant classification rely on a wide-array of populational, evolutionary, physico-chemical, structural, and or molecular annotations and properties for the classification of variants, existing computational methods and systems do not employ information pertaining to the impacts of molecular variants on cellular biology. As a consequence, such computational methods are unable to capture phenotypic impacts acting through variation in molecular properties within cells or variation in cellular populations and cellular heterogeneity.

Second, existing large-scale functional assays and solutions that are capable of assaying the activity of thousands of molecular variants provide activity measurements along a single dimension per molecular variant, and often require a priori knowledge or assumptions of the mechanism of action through which molecular variants exert phenotypic impacts.

Owing to these limitations, while conventional computational methods and systems for variant classification can access data across a multiplicity of annotations and parameters, these conventional approaches have demonstrably poor performance in classification (and regression) tasks for the phenotypic impact of molecular variants. Similarly, these conventional approaches require a priori knowledge or assumptions of the mechanism of action (and hence relevant molecular functions to assay), which limits their application to well-characterized functional elements (e.g., genes). This further precludes their application to poorly understood disease-associated genes. Finally, these conventional approaches require significant development and customization to assay each molecular function and each functional element.

In embodiments herein, a technological solution to overcome these technological problems involves data structures providing multi-dimensional characterization of cells and cellular populations harboring specific genotypes (e.g., molecular variants) in one or more functional elements (e.g., genes) and in one or more contexts (e.g., cell-types, drug treatments, genotypic backgrounds). Such data structures enable systems and methods for statistical learning to achieve improved accuracy in the classification tasks pertaining to the phenotypic impacts of genotypes (e.g., molecular variants or combinations thereof).

Embodiments herein enable robust, scalable, multi-dimensional classification of molecular variants (and combinations thereof) across a wide-array of functional elements and phenotypes through the acquisition of hundreds to tens of thousands (˜10²-10⁴) of molecular measurements per model system (e.g., cell), the construction of molecular profiles for tens to thousands (˜10¹-10³) of model systems per molecular variant, thousands (˜10³) of molecular variants per functional element (e.g., genes), and a single or a multiplicity of functional elements in parallel.

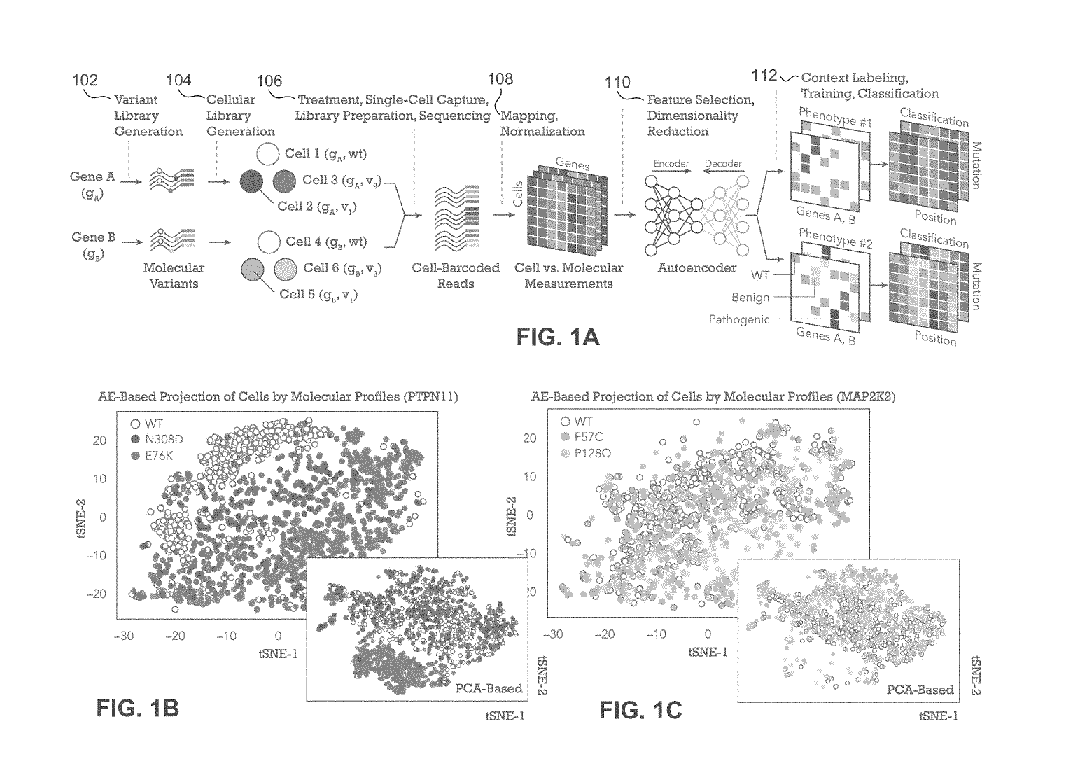

As illustrated in FIG. 1A, an embodiment of the present disclosure integrates Variant Library Generation 102 and Cellular Library Generation 104 methods for high-throughput mutagenesis and cellular engineering techniques to create compendiums of model systems (e.g., cells) harboring distinct molecular variants in target functional elements (e.g., genes). The embodiment provides Treatment, Single-Cell Capture, Library Preparation, Sequencing 106 methods utilizing cellular, molecular biology, and genomics techniques and technologies for treatment and capture of model systems, preparation of libraries of molecular entities, and for measuring diverse molecular entities (e.g., transcripts) within model systems. The embodiment provides Mapping, Normalization 108 bioinformatics, computational biology, and statistical techniques for mapping, quantifying, and normalizing associations between molecular variants, model systems, and molecular entities within each model system. The embodiment provides Feature Selection, Dimensionality Reduction 110 and Context Labeling, Training, Classification 112 statistical (e.g., machine) learning, distributed and high-performance computing, systems biology, population and clinical genomics techniques for label generation, feature selection, dimensionality reduction, training, and classification of molecular variants.

In some embodiments, the present disclosure describes the use of these series of methods and technologies of FIG. 1A to determine the phenotypic impacts of molecular variants identified within a biological sample. In some embodiments, the present disclosure describes the introduction of molecular variants into one or more functional elements within a model system. The model system can include single-cells, cellular compartments, subcellular compartments, or synthetic compartments. In some embodiments, the present disclosure describes the determination of molecular scores or phenotype scores of the single-cells, the cellular compartments, the subcellular compartments, or the synthetic compartments. In some embodiments, the present disclosure describes the identification of molecular variants within the single-cells, the cellular compartments, the subcellular compartments, or the synthetic compartments. As would be appreciated by a person of ordinary skill in the art, various methods can be utilized to identify molecular variants within the single-cells, the cellular compartments, the subcellular compartments, or the synthetic compartments. This may be on the basis of molecular measurements of the single-cells, the cellular compartments, the subcellular compartments, or the synthetic compartments. In some embodiments, the present disclosure describes the determination of molecular signals or phenotype signals associated with individual molecular variants on the basis of molecular scores or phenotype scores, respectively, from the single-cells, the cellular compartments, the subcellular compartments, or the synthetic compartments associated with specific molecular variants. In some embodiments, the present disclosure describes the determination of population signals associated with molecular variants on the basis of molecular scores or phenotype scores of the single-cells, the cellular compartments, subcellular compartments, or the synthetic compartments associated with specific molecular variants.

In some embodiments, the present disclosure describes the determination of functional scores or functional classifications of molecular variants by applying statistical (e.g., machine) learning approaches that associate molecular signals, phenotype signals, or population signals with the phenotypic impacts of the molecular variants. In some embodiments, the present disclosure describes the determination of evidence scores or evidence classifications of the molecular variants based on functional scores, functional classifications, predictor scores, predictor classifications, hotspot scores, or hotspot classifications. In some embodiments, the present disclosure describes the determination of the phenotypic impacts of the molecular variants identified within biological samples on the basis of the functional scores, the functional classifications, the evidence scores, or the evidence classifications of the identified molecular variants.

Embodiments herein integrate methods, techniques, and technologies from a multiplicity of domains. While statistical, machine learning techniques leveraging single-cell molecular measurements have been developed and applied for the classification of model systems (e.g., cells) originating from tens (e.g., less than 10²) of different tissues or developmental stages, the requirements for achieving accurate genotype-specific (e.g. molecular variant-specific) classifications among thousands of cells with subtle differences—such as a single nucleotide difference in a genomic background defined by greater than 3×10⁹ nucleotides—within the same cell-lines, tissues, or developmental stages, can present substantial challenges.

The present disclosure provides Deep Mutational Learning (DML) system, apparatus, device, method and/or computer program product embodiments, and/or combinations and sub-combinations thereof for overcoming challenges in the identification (e.g., classification) of the phenotypic impact of molecular variants identified in subjects on the basis of biological signals assayed in single and populations of model systems (e.g., cells).

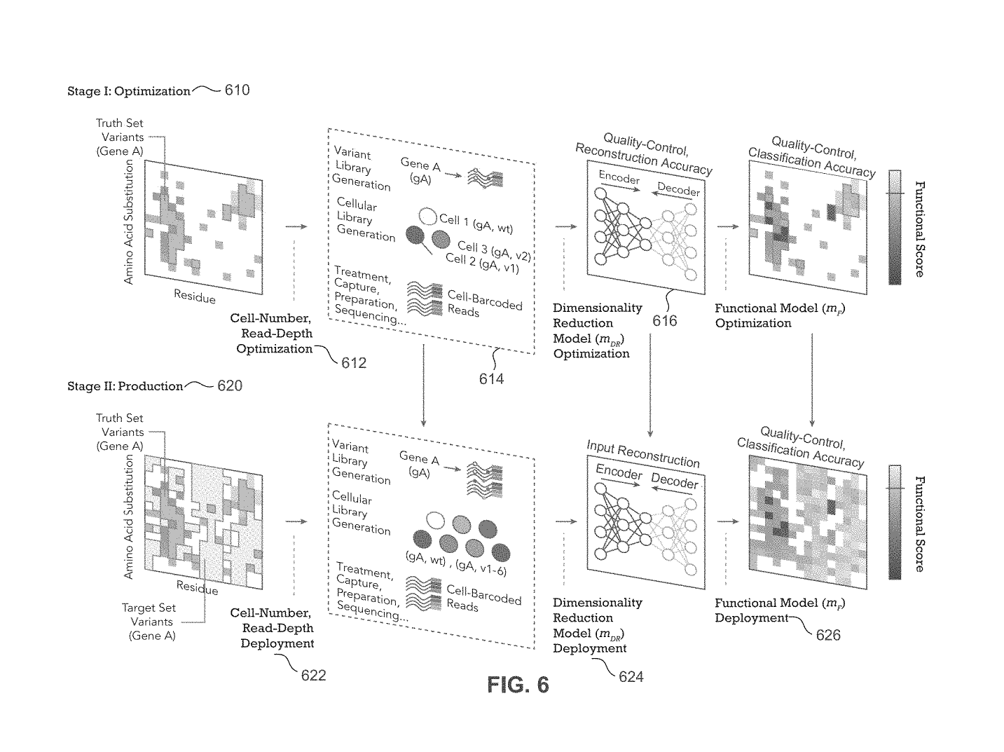

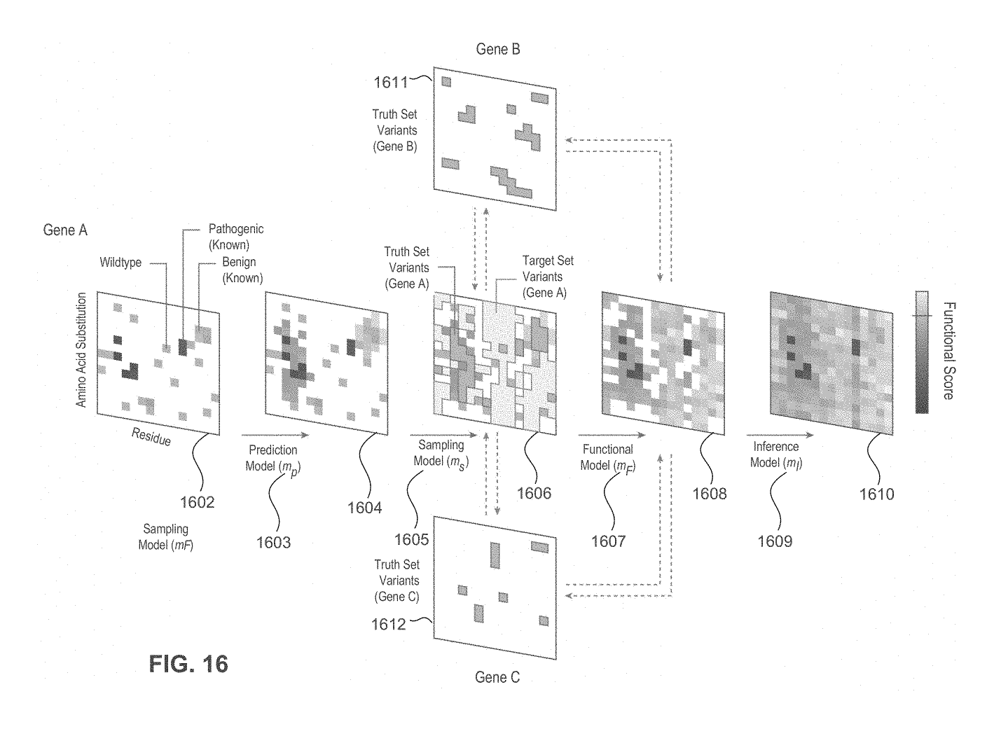

The present disclosure provides system, apparatus, device, method and/or computer program product embodiments, and/or combinations and sub-combinations thereof that improve cost-efficiency in the classification of molecular variants through (i) the directed deployment of DML processes and systems with lower-cost prediction models (see FIG. 16), and (ii) tiered deployment of DML processes and systems that allow robust reconstruction of molecular signals at reduced costs (see FIG. 6).

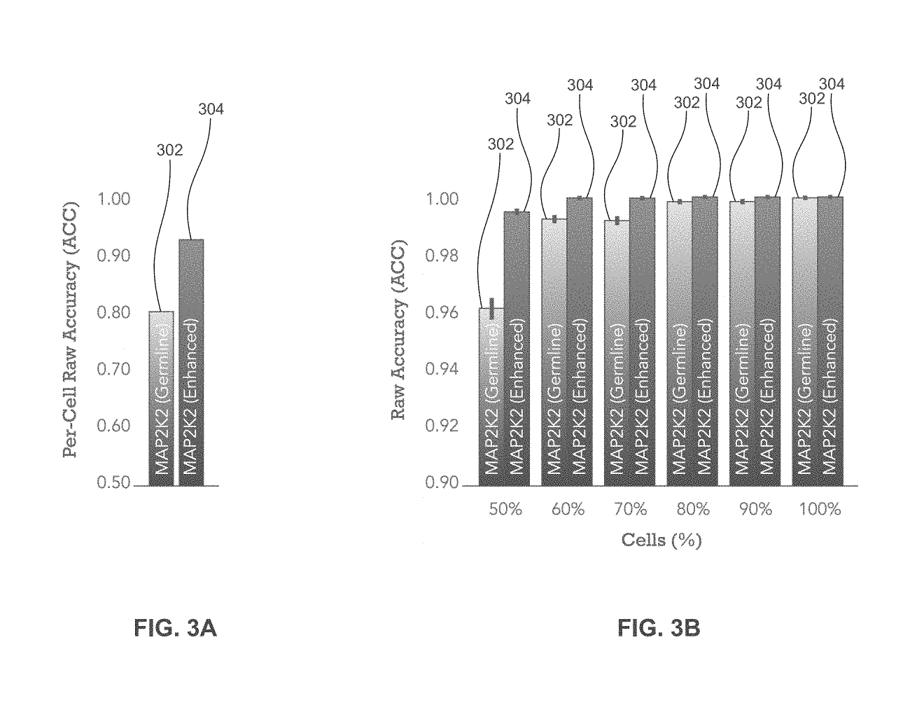

The present disclosure provides system, apparatus, device, method and/or computer program product embodiments, and/or combinations and sub-combinations thereof that improve the scalability and performance across functional elements (e.g., genes) through DML processes and systems that leverage information between functional elements (see FIGS. 3A and 3B).

The present disclosure provides system, apparatus, device, method and/or computer program product embodiments, and/or combinations and sub-combinations thereof for assessing the phenotypic impacts (e.g., pathogenicity, functionality, or relative effect) of one or more molecular (e.g., genotypic) variants in one or more (e.g., coding or non-coding) functional elements (e.g., protein-coding genes, non-coding genes, molecular domains such as protein or RNA domains, promoters, enhancers, silencers, regulatory binding sites, origins of replication, etc.) in the (e.g., nuclear, mitochondrial, etc.) genome(s), or their derivative molecules. As would be appreciated by a person of ordinary skill in the art, a molecular variant may be a genotypic (e.g., sequence) variant such as a single-nucleotide variant (SNV), a copy-number variant (CNV), or an insertion or deletion affecting a coding or non-coding sequence (or both) in the nuclear, mitochondrial, or episomal genome-natural or synthetic. As would be appreciated by a person of ordinary skill in the art, a molecular variant may also be a single-amino acid substitution in a protein molecule, a single-nucleotide substitution in a RNA molecule, a single-nucleotide substitution in a DNA molecule, or any other molecular alteration to the cognate sequence of a polymeric biological molecule.

In some embodiments, the classification (or regression) may relate to (e.g., likely) disease-causing (e.g., pathogenic) and neutral (e.g., benign) variants for disorders with genetic components, or predictions of the severity thereof, on the basis of the molecular variants identified within a biological sample or record thereof of a subject. In some other embodiments, the classification (or regression) may relate to molecular impacts (e.g., loss-of-function, gain-of-function or neutral) on the basis of molecular variants of probable molecular consequence (e.g., nonsense or insertion and deletion mutations) and probable molecular neutrality (e.g., synonymous). In some other embodiments, the classification (or regression) may relate to variation in the response to therapeutic treatments (e.g., chemical, biochemical, physical, behavioral, digital, or otherwise) on the basis of molecular variants identified within a biological sample or record thereof of a subject. In some embodiments, phenotypic impacts may refer to phenotype classes (e.g., neutral, pathogenic, benign, high-risk, low-risk, positive response variants, negative response variants) and phenotype scores (e.g., a probability of developing specific clinical and non-clinical phenotypes, the levels of metabolites in blood, and the rate at which specific compounds are absorbed or metabolized).

In some embodiments, the present disclosure provides systems and methods for modeling the diversity and prevalence of phenotypic properties within a population on the basis of the diversity and prevalence of molecular variants in representative populations. In some embodiments, the present disclosure provides systems and methods for modeling the diversity and prevalence of phenotypic properties within a population on the basis of the phenotypic impacts of molecular variants—with known or expected diversity and prevalence—where the phenotypic impacts may be modeled from one or more molecular signals, phenotype signals, or population signals, previously associated with variants in an in vivo or in vitro functional model system. In some embodiments, such modeling may be used to inform on the diversity and prevalence of mechanisms of drug-resistance in a population.

In some embodiments, the present disclosure describes the use of models of the diversity and prevalence of phenotypic properties within a population of individuals (e.g., as informed by the phenotypic impacts of molecular variants modeled from one or more molecular signals, phenotype signals, or populations signals in a functional model system) to construct cohorts of subjects (e.g., patients) and to investigate the efficacy of therapeutic and non-therapeutic interventions.

In some embodiments, the present disclosure provides systems and methods for the classification (or regression) of the phenotypic impact of molecular variants on the basis of functional scores or functional classifications derived from one or more molecular signals, phenotype signals, or population signals associated with variants as assayed in a functional model system. In some embodiments, molecular variants may be functionally modeled within cells, cellular compartments or synthetic compartments as in vivo or in vitro model systems.

In some embodiments, the molecular variants modeled (e.g., in vivo or in vitro) may be identified directly within the nucleic acid sequence of the functional elements modeled via library preparation, sequencing, and characterization of nucleic acids or nucleic acid fragments within single-cells, cellular compartments, subcellular compartments, or synthetic compartments (e.g., collectively termed model systems). In some other embodiments, the molecular variants modeled (e.g., in vivo or in vitro) may be inferred from barcode sequences associated with individual variants in the functional elements via library preparation, sequencing, and characterization of nucleic acids or nucleic acid fragments within model systems (e.g., single-cells, cellular compartments, subcellular compartments, or synthetic compartments), using a pre-assembled database of associated barcodes and variants. As would be appreciated by a person of ordinary skill in the art, molecular variants may be produced via a diversity of techniques, such as direct (e.g., chemical) synthesis, error-prone PCR, oligonucleotide-directed mutagenesis, nicking mutagenesis, or Saturation Genome Editing (SGE), among others (Firnberg et al. 2012; Kitzman et al. 2014; Wrenbeck et al. 2016; and Findlay et al. 2014). As would be appreciated by a person of ordinary skill in the art, variant libraries can be then introduced (e.g., added) into model systems (e.g., cells, cellular compartments, subcellular compartments, or synthetic compartments) using a variety of approaches, such as but not limited to homologous recombination (e.g., Cas9-mediated or Adenovirus-mediated), site-specific recombination (e.g., Flp-mediated), or viral transduction (eg., lentiviral-mediated) (Findlay et al. 2018; Wissink et al. 2016; and Macosko et al. 2015).

In some embodiments, functional scores and functional classifications associated with individual molecular variants may be derived from measurements of molecules and or chemical modifications present within in vivo or in vitro model systems harboring the variant within the functional element, including but not limited to DNA, RNA, and protein molecules or modifications thereof. For example, in some embodiments, measurements or models of molecular signals, cellular signals, or population signals may be made and used to learn the functional scores and or functional classifications. In some embodiments, the functional scores and functional classifications may be derived from molecular measurements obtained via nucleic acid barcoding, isolation, enrichment library preparation, sequencing, and characterization of a plurality of nucleic acids or nucleic acid fragments within single-cells, cellular compartments, subcellular compartments, or synthetic compartments including, but not limited to, RNA molecules, genomic DNA, chromatin-associated DNA, protein-associated DNA, accessible DNA fragments, or chemically-modified nucleic acids. In some embodiments, these procedures may utilize molecular barcoding techniques to uniquely identify or associate nucleic acids, nucleic acid fragments, or nucleic acid sequences stemming from individual single-cells, cellular compartments, subcellular compartments, or synthetic compartments (Macosko et al. 2015; Buenrostro et al. 2015; Cusanovich et al. 2015; Dixit et al. 2016; Adamson et al. 2016; Jaitin et al. 2016; Datlinger et al. 2017; Zheng et al. 2017; Cao et al. 2017). These methods may build on developments from the field of single-cell genomics (Schwartzman and Tanay 2015; Tanay and Regev 2017; Gawad et al. 2016). In some embodiments, the systems and methods of the present disclosure may apply methods for single-cell RNA sequencing to derive molecular measurements from single-cells, cellular compartments, subcellular compartments, or synthetics compartments. These methods include but are not limited to single-cell sequencing library generation, high-throughput nucleic acid sequencing, sequencing read quality control, barcode identification (e.g., of single-cell, cellular compartment, subcellular compartment, or synthetic compartment) and quality control, sequencing read unique molecular barcode identification and quality control, sequencing read alignments, as well as read alignment filtering and quality control. In some embodiments, molecular measurements may correspond to locus-specific measurements of gene expression (e.g., RNA transcript abundance), protein abundance or modifications (e.g., phospho-protein abundance), chromatin accessibility (e.g., nucleosome occupancy), epigenetic modification (e.g., DNA methylation), regulatory activity (e.g., transcription factor binding), post-transcriptional processing (e.g., splicing), post-translational modification (e.g., ubiquitination), mutation burden (e.g., count), mutation rate (e.g., frequency), mutation signatures (e.g., count or frequency per type of mutation), or various other types of measurements of molecules within single-cells, cellular compartments, subcellular compartments, or synthetic compartments as would be appreciated by a person of ordinary skill in the art. In some embodiments, the present disclosure describes systems and methods for augmenting the quality of the molecular measurements for specific target genes and functional elements via the use targeted enrichment or targeted capture techniques—via hybridization- or amplicon-based techniques and probes—either before, during or after single-cell RNA library processing.

In some embodiments, molecular measurements from single-cells, cellular (or subcellular) compartments or synthetic compartments may be utilized to derive multi-locus measurements of molecular processes. For example, these measurements of molecular processes may include multi-locus measurements of gene expression, chromatin accessibility, epigenetic modification, regulatory activity, transcriptional activity, translational activity, signaling activity, signaling activity, pathway activity, mutation burden, mutation rate, mutation signatures, and various other measurements as would be appreciated by a person of ordinary skill in the art.

In some embodiments, molecular measurements and molecular processes from single-cells, cellular (or subcellular) compartments or synthetic compartments may be utilized to derive global (e.g., pan-locus or locus-independent) measurements of molecular features. For example, these measurements of molecular features may include global measurements of gene expression, chromatin accessibility, epigenetic modification, regulatory activity, transcriptional activity, translational activity, signaling activity, signaling activity, pathway activity, mutation burden, mutation rate, mutation signatures, and various other measurements as would be appreciated by a person of ordinary skill in the art.

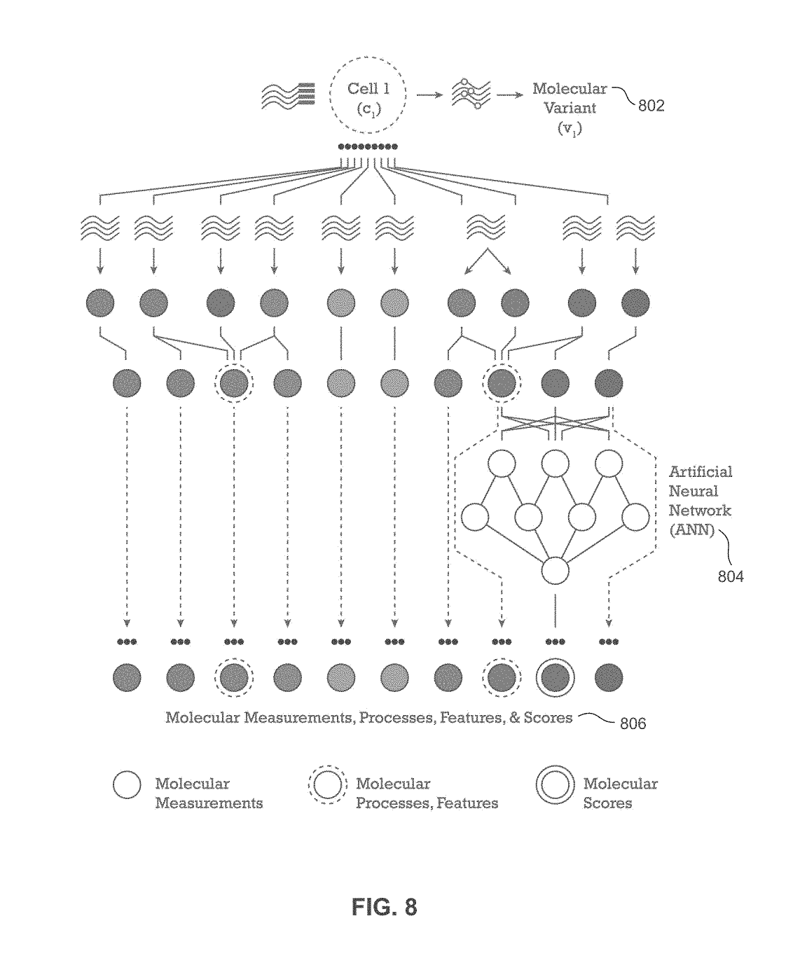

In some embodiments, molecular measurements, molecular processes, or molecular features of single-cells, cellular compartments, subcellular compartments, or synthetic compartments may serve directly as (e.g., lower-order) molecular scores. In some embodiments, a (e.g., higher-order) molecular score may be derived by applying pre-existing models that associate multiple lower-order (e.g., lower-order) molecular scores (e.g., molecular measurements, molecular processes, or molecular features) to regulatory, signaling, pathway, processing, cell-cycle activities, alterations, defects, or states. In some embodiments, such methods may apply gene set enrichment analysis or other derivative methods as would be appreciated by a person of ordinary skill in the art. In some embodiments, as illustrated in FIG. 8, the molecular measurements, molecular processes, molecular features, or (e.g., lower-order) molecular scores 806 from single-cells, cellular compartments, subcellular compartments, or synthetic compartments harboring the same molecular variants 802 may be fed through a series of artificial neuron layers (e.g., convolutional or perceptron layers) in an Artificial Neural Network 804 (ANN) to derive increasingly complex (e.g., higher-order) molecular scores 806, and generate autoencoders with learned features. In some embodiments, methods for computing molecular scores, such as pathway level analyses, may be used to preserve information of biological function while allowing for dimensionality reduction.

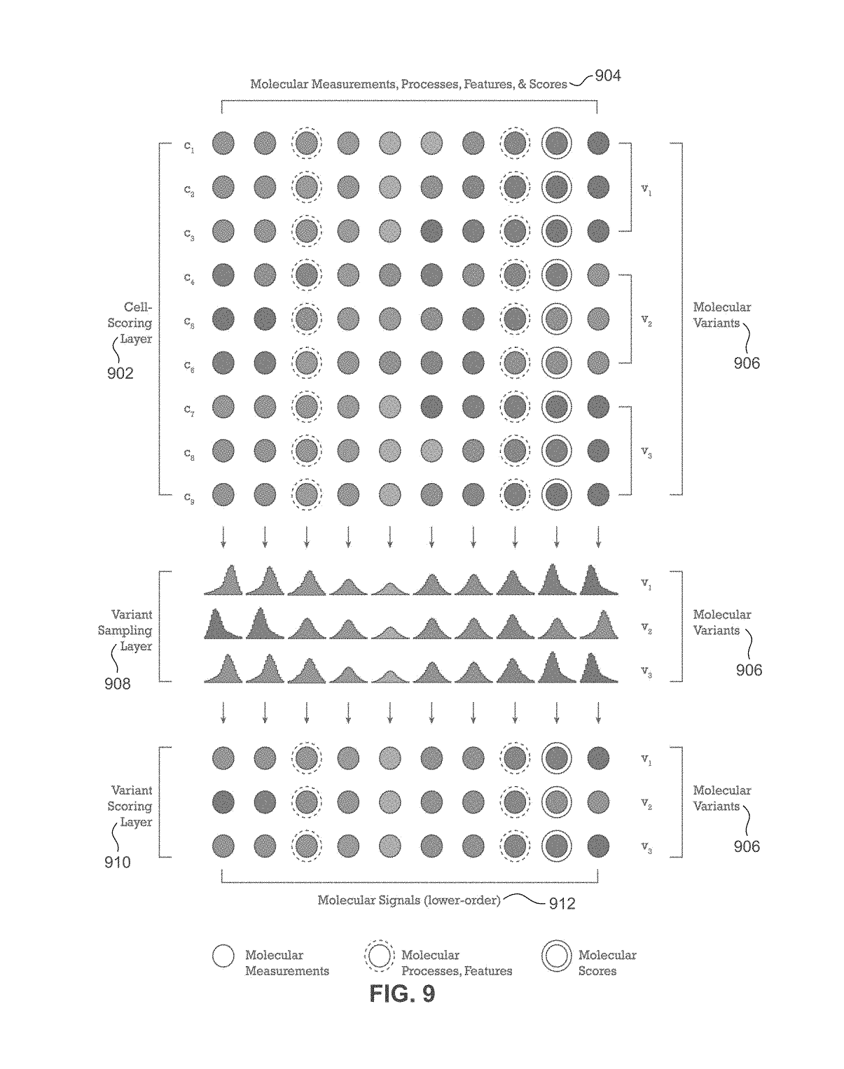

In some embodiments, as illustrated in FIG. 9, a database of molecular scores may be constructed via a cell scoring layer 902 from a plurality of individual single-cells, cellular compartments, subcellular compartments, or synthetic compartments. In some embodiments, the molecular scores from a plurality of single-cells, cellular compartments, subcellular compartments, or synthetic compartments, harboring the same molecular variants 906 (e.g., v₁, v₂, and v₃) may be accessed with a variant sampling layer 908 and analyzed in a variant scoring layer 910 to derive (e.g., directly measure or model) summary statistics relating to the tendency (e.g., mean, median, mode), dispersion (e.g., variance, standard deviation), shape (e.g., skewness, kurtosis), probability (e.g., quantiles), range (e.g., confidence interval, minimum, maximum), error (e.g., standard error), or covariation (e.g., covariance) of molecular scores associated with individual molecular variants. In some embodiments, as illustrated in FIG. 9, summary statistics relating to the tendency, dispersion, shape, range, or error of molecular scores may be used to create a database of (e.g., quality-controlled) molecular signals 912 associated with individual molecular variants 906. In some embodiments, molecular measurements, molecular processes, molecular features, and molecular scores 904 may be properties of individual single-cells, cellular compartments, subcellular compartments, or synthetic compartments. In some embodiments, molecular signals may be a property of molecular variants.

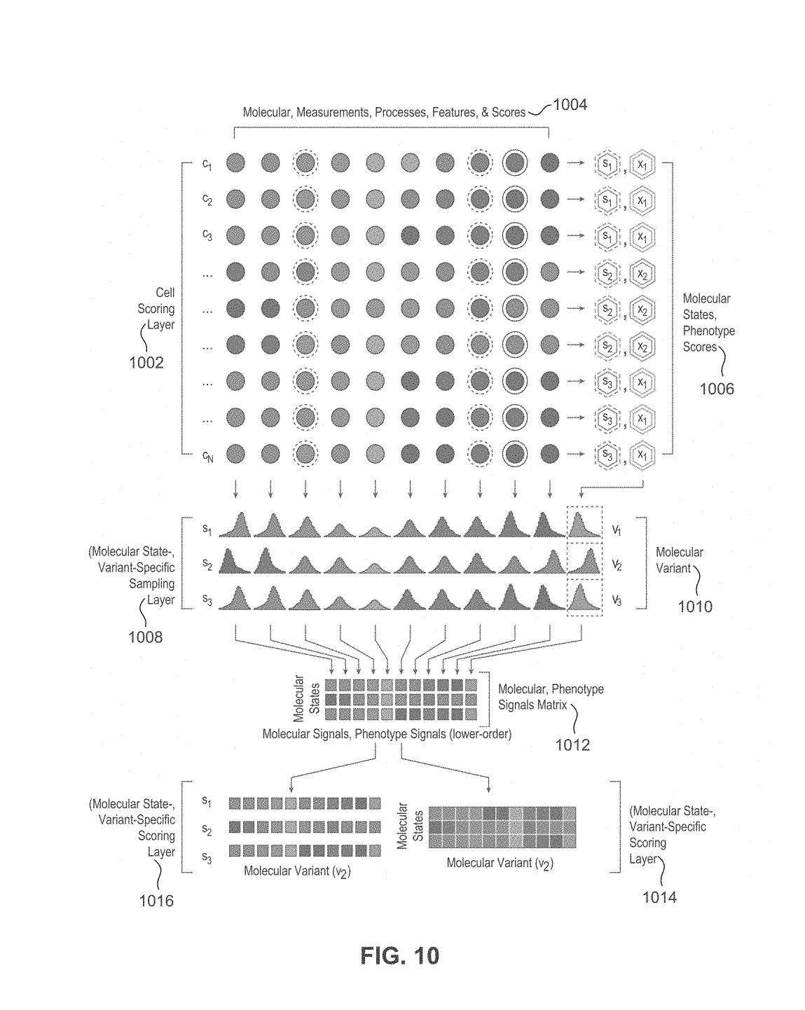

As would be appreciated by a person of ordinary skill in the art, the molecular measurements, processes, features, and scores from model systems (e.g., single-cells, cellular compartments, subcellular compartments, or synthetic compartments) may define or correspond to distinct molecular states or specific subpopulations of model systems (e.g., single-cells, cellular compartments, subcellular compartments or synthetic compartments) with similar molecular properties. As would be appreciated by a person of ordinary skill in the art and as shown in FIG. 10, a cell scoring layer 1002 can be applied to determine the molecular states, phenotype scores 1006 (e.g., s₁, s₂, s₃) of model systems on the basis of a variety of methods.

For example, the molecular states of model systems can be identified on the basis of cell-cycle signatures derived from gene-expression molecular scores (Macosko et al. 2015). As would be appreciated by a person of ordinary skill in the art, molecular states can be derived via scoring using previously-derived models—for example, scoring gene-expression signatures of previously characterized molecular states such as gene-expression signatures reflecting distinct phases of the cell-cycle previously characterized in chemically synchronized cells (Whitfield et al. 2002). As would be appreciated by a person of ordinary skill in the art, molecular states may also be derived via scoring using internally-derived models from partitions of model systems within which characteristic correlations between molecular signals can be detected or expected (e.g., as is the case with gene expression variation throughout distinct stages of cell-cycle). As would be appreciated by a person of ordinary skill in the art, the internally-derived models may be generated using a variety of statistical techniques (e.g., machine learning techniques).

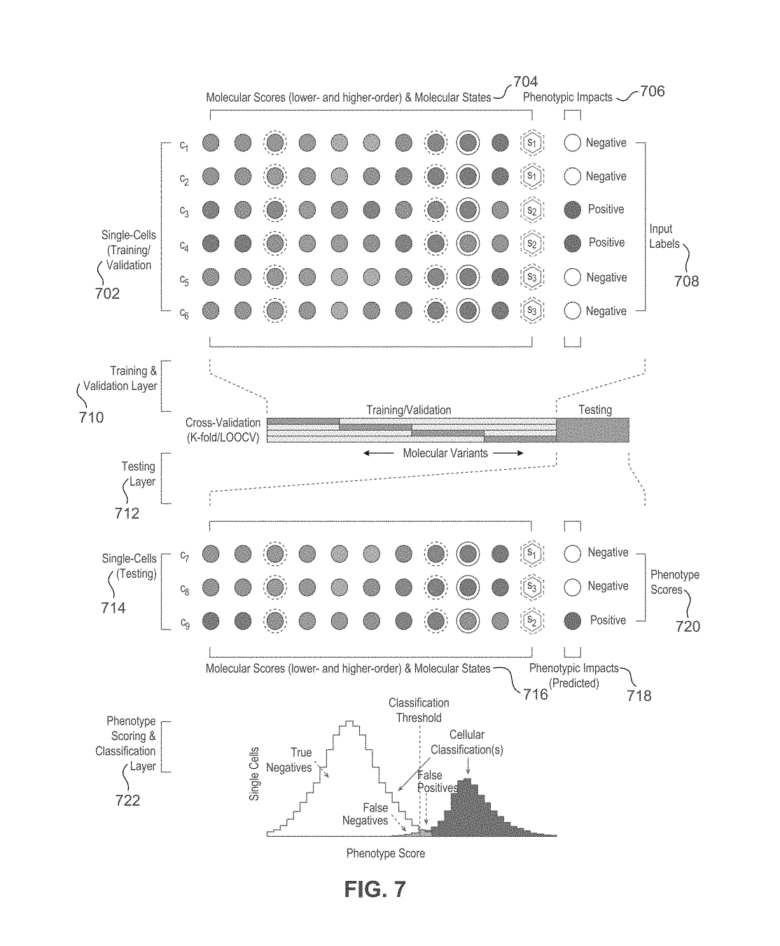

In some embodiments, as illustrated in FIG. 7, the present disclosure provides systems and methods to generate a Phenotype Model (m_{P}) for deriving phenotype scores through the use of statistical techniques (e.g., machine learning techniques) that associate molecular scores and molecular states of model systems (e.g., single-cells, cellular compartments, subcellular compartments or synthetic compartments) with the phenotypic impacts of molecular variants within each model system. Whereas molecular scores can relate directly to molecular, biological, or physical properties within individual model systems, phenotype scores can describe the (e.g., likely) phenotypic associations of molecular variants. In some embodiments, the phenotype scores are derived by applying supervised learning techniques to associate the phenotypic impacts (e.g., labels) of molecular variants within model systems with the molecular scores or molecular states (e.g., features) of model systems.

In some embodiments, a Phenotype Model (m_{P}) and database of phenotype scores (or phenotype classifications) is generated by accessing a database of features describing (e.g., lower- and higher-order) molecular scores and molecular states 704 of single-cells 702, and input labels 708 (e.g., a database) describing the phenotypic impact 706 of molecular variants identified within single-cells 702. In some embodiments, a training/validation layer 710 generates and quality-controls Phenotype Models (m_{P}) that can predict the phenotypic impact 706 of individual single-cells 702. In some embodiments, a database of features describing the molecular scores and molecular states 716 of single-cells (testing) 714 are provided to the generated Phenotype Models (m_{P}) to calculate and create a database of phenotype scores 720 describing the predicted phenotypic impact 718 of molecular variants in single-cells (testing) 714. As would be appreciated by a person of ordinary skill in the art, the performance (e.g. accuracy) of the predicted phenotypic impacts 718 in each cell (e.g., phenotype scores 720) can be determined against the known phenotypic impact of molecular variants in single-cells (testing) 714 within a testing layer 712. As would be appreciated by a person of ordinary skill in the art, the Phenotype Models (m_{P}) can be applied to pre-compute or compute, on demand, the phenotype scores of single cells not included in training, validation, or testing. In some embodiments, such scoring and evaluation can occur in a phenotype scoring and classification layer 722. Phenotype scoring and classification layer 722 can examine the phenotype impact classification accuracy permitted on the basis of phenotype scores 720.

In some embodiments, summary statistics relating to the tendency, dispersion, shape, range, or error of phenotype scores may be used to create a database of (e.g., quality-controlled) phenotype signals associated with individual molecular variants.

In some embodiments, and as illustrated in FIG. 10, the present disclosure describes the use of molecular state-specific molecular signals for subsequent rounds of unsupervised and supervised learning, in either the generation of molecular state-specific models or multi-state models. In some embodiments and as illustrated in FIG. 10, the present disclosure describes the use of a molecular state-, variant-specific sampling layer 1008 to access the molecular measurements, processes, features, and scores 1004 and the molecular states, phenotype scores 1006 of model systems with specific molecular variants 1010 (e.g., v₁, v₂, v₃) and in specific molecular states, with characteristic phenotype scores, or combinations thereof. In some embodiments, the molecular measurements, processes, features, and scores 1004 or the molecular states, phenotype scores 1006 may be pre-computed or computed on demand by a cell scoring layer 1002. In some embodiments, data, summary statistics, descriptive statistics (e.g., univariate, bivariate, or multivariate analysis), inferential statistics, Bayesian inference models (e.g., variational Bayesian inference models), Dirichlet processes, or other models of the data accessed by the molecular state-, variant-specific sampling layer 1008 are used to construct a molecular, phenotype signals matrix 1012, describing molecular signals and phenotype signals in each molecular state for each molecular variant.

In some embodiments, the molecular, phenotype signals matrix 1012 may be pre-computed or computed on demand. In some embodiments, the molecular, phenotype signals matrix 1012 may be pre-computed or computed on demand by a molecular state, variant-specific scoring layer 1016 yielding matrices that are molecular state-specific. In some embodiments, the molecular, phenotype signals matrix 1012 may be pre-computed or computed on demand by a multi-state, variant-specific scoring layer 1014, yielding matrices that contain data from multiple molecular states.

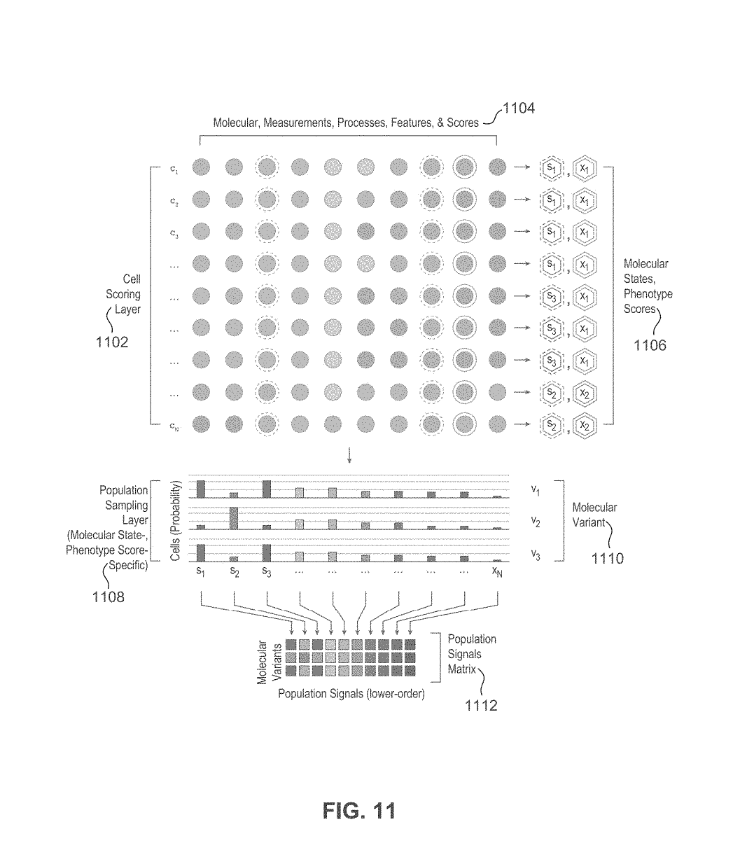

In some embodiments, as illustrated in FIG. 11, the present disclosure provides methods for characterizing the distribution of cells with specific molecular variants across molecular states (e.g., sub-populations) or phenotype scores 1106, as produced by a cell scoring layer 1102 using molecular measurements, processes, features and scores 1104 as inputs. These molecular states (e.g., sub-populations) or phenotype scores may be associated with, but not limited to, subpopulations of cells defined by (a) characteristic levels of or correlations between molecular signals (e.g., cyclin dependent kinases during the cell-cycle stage), whether determined by the application of pre-existing or internally-derived models, (b) characteristic levels of or correlations between phenotype scores, or (c) unsupervised or supervised machine learning methods, including but not limited to dimensionality reduction techniques, examples of which include but are not limited to Principal Component Analysis (PCA), Independent Component Analysis (ICA), and t-Stochastic Neighbor Embedding (tSNE). In some embodiments, as illustrated in FIG. 11, for each individual molecular variant 1110, a population sampling layer 1108 produces metrics of the relative representation (e.g., distribution, probability, etc.) of cells across molecular states (e.g., the proportion or the probability of variant-harboring cells residing in a molecular state) or phenotype scores (e.g., the proportion or the probability of variant-harboring cells having a particular score), and may serve to provide a population signals matrix 1112 describing how molecular variants affect cells at the population-level. The population signals matrix 1112 may contain a plurality of population signals for a plurality of molecular variants.

In some embodiments, subsampling of molecular measurements, molecular processes, molecular features, molecular scores, or phenotype scores from model systems (e.g., single-cells, cellular compartments, subcellular compartments, or synthetic compartments) harboring the same molecular variant may be applied to generate independent or disjoint estimates of summary statistics relating to the tendency, dispersion, shape, probability, range, covariation, or error of molecular measurements, molecular processes, molecular features, or molecular scores or phenotype scores associated with individual molecular variants.

In some embodiments, independent or disjoint estimates of summary statistics relating to the tendency, dispersion, shape, probability, range, covariation, or error of molecular measurements, molecular processes, molecular features, molecular scores or phenotype scores may be used to create a database of (quality-controlled) independent or disjoint estimates of molecular signals or phenotype signals associated with individual molecular variants. As would be appreciated by a person of ordinary skill in the art, independent or disjoint estimates of molecular signals or phenotype signals can be used to create a database of (quality-controlled) molecular or phenotype signals associated with individual molecular variants.

In some embodiments, the present disclosure describes systems and methods for deriving independent or disjoint estimates of summary statistics relating to the tendency, dispersion, shape, probability, range, covariation, or error of molecular measurements, molecular processes, molecular features, or molecular scores or phenotype scores associated with individual molecular variants within subpopulations of model systems (e.g., single-cells, cellular compartments, subcellular compartments, or synthetic compartments) from specific molecular states. As would be appreciated by a person of ordinary skill in the art, these methods may leverage a plurality of statistical techniques (e.g., machine learning techniques).

In some embodiments, molecular state-specific independent or disjoint estimates of summary statistics relating to the tendency, dispersion, shape, probability, range, covariation, or error of molecular measurements, molecular processes, molecular features, molecular scores or phenotype scores may be used to create a database of (e.g., quality-controlled) molecular state-specific, independent and disjoint estimates of molecular signals and phenotype signals associated with individual molecular variants in specific molecular states.

In some embodiments, independent or disjoint estimates of summary statistics relating to the tendency, dispersion, shape, probability, range, covariation, or error of population signals associated with individual molecular variants may be used to create a database of (e.g., quality-controlled) population signals associated with individual molecular variants.

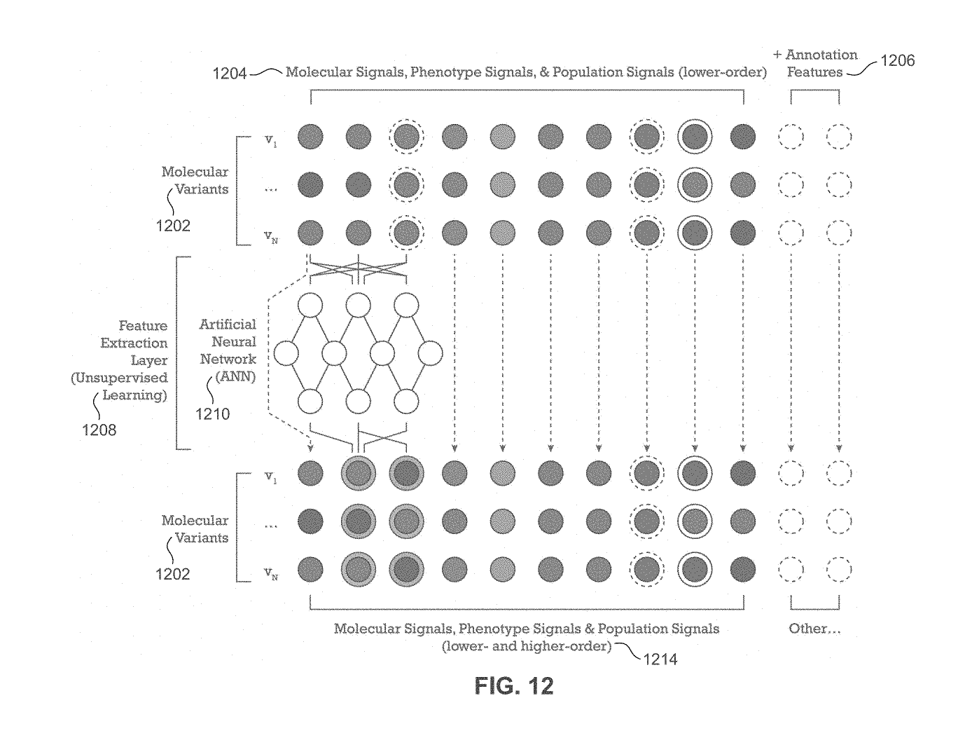

In some embodiments, as illustrated in FIG. 12, the present disclosure provides systems and methods leveraging a feature extraction layer 1208 (e.g., unsupervised learning techniques) for the identification of higher-order molecular signals, phenotype signals, or population signals from lower-order molecular signals, phenotype signals, or population signals 1204 associated with individual molecular variants 1202, including but not limited to feature learning (or representation learning) techniques deploying Artificial Neural Networks (ANNs) 1210 to generate auto-encoders capable of leveraging subjacent associations to yield higher-order representations of lower-order molecular, phenotype, or population signals. In some embodiments, these methods allow the construction of databases lower- and higher-order molecular signals, phenotype signals, and population signals 1214. In some embodiments, the feature extraction layer 1208 may access or receive data from annotation features 1206, in addition to the lower-order molecular signal, phenotype signals, or population signals 1204. In some embodiments, the annotation features 1206 may encompass a plurality of independent (e.g., non-assayed) features (e.g., evolutionary, population, functional (e.g., annotation-based), structural, dynamical, and physicochemical features associated with variants, genomic coordinates, transcript (e.g., RNA) coordinates, translated (e.g., protein) coordinates, amino acids, and various others as would be appreciated by a person of ordinary skill in the art), describing changes associated with the changes in genotype (e.g., sequence, molecular variants, etc.).

In some embodiments, the present disclosure describes the use of molecular state-specific, lower-order molecular signals or phenotype signals for the derivation of molecular state-specific higher-order molecular signals or phenotype signals. In some embodiments, the present disclosure describes the use of multi-state matrices of lower-order molecular, phenotype, or population signals to derive multi-state higher-order molecular, phenotype, or population signals, leveraging structured relationships between molecular signals across molecular states, such as structured gene expression patterns (e.g., molecular signals) across cell-cycle stages (e.g., molecular states). In some embodiments, the present disclosure describes the use of Convolutional Neural Networks (CNNs) to learn patterned-associations in molecular, phenotype, or population signals (and annotation features) across molecular states.

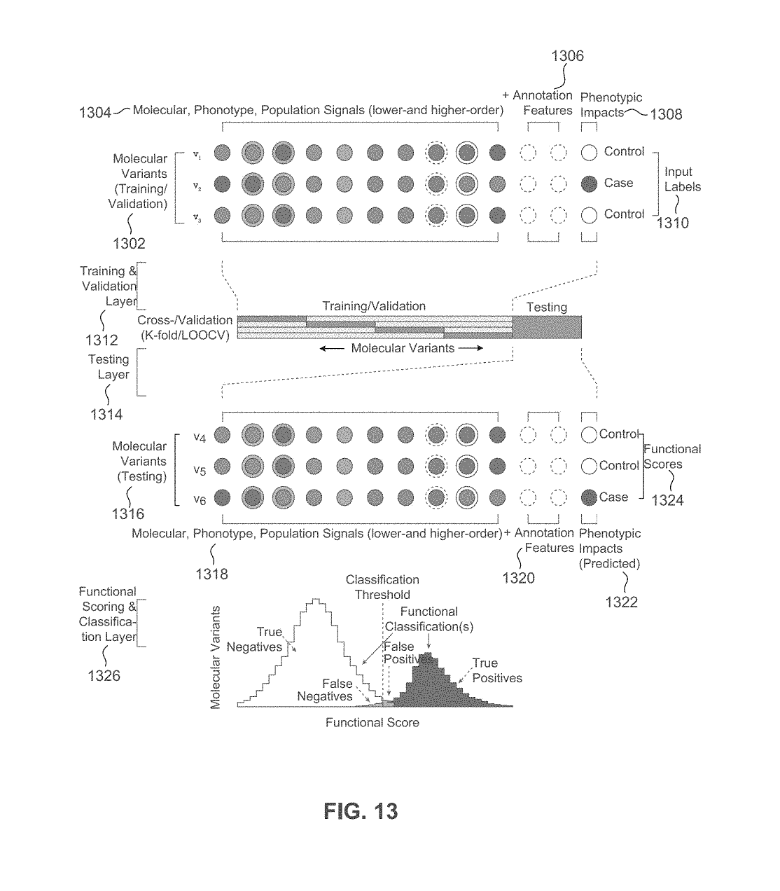

In some embodiments, and as illustrated in FIG. 13, the present disclosure provides systems and methods for deriving functional scores and functional classifications via statistical (e.g., machine) learning to generate a Functional Model (m_{F}) that associates molecular, phenotype, or population signals (e.g., features)—a single or plurality of molecular measurements, molecular processes, molecular features, and molecular scores—with phenotypic impacts (e.g., labels) of molecular variants via regression and classification techniques, respectively.

In some embodiments, a Functional Model (m_{F}) and a database of functional scores (or functional classifications) is generated by accessing a database of features describing molecular (e.g., lower-order or higher-order), phenotype, or population signals 1304 of molecular variants 1302 for training/validation, and a set of input labels 1310 (e.g., a database) describing the phenotypic impacts 1308 of molecular variants 1302. The generating is further performed by applying statistical (e.g., machine) learning techniques to associate molecular, phenotype, or population signals 1304 (e.g., features) to phenotypic impacts (e.g., labels).

In some embodiments, a training/validation layer 1312 performs training and validation to generate quality-control Functional Models (m_{F}) that can predict the phenotypic impacts 1308 of molecular variants 1302. In some embodiments, training/validation layer 1312 can deploy cross-validation techniques, such as, but not limited to, K-fold or Leave-One-Out Cross-Validation (LOOCV). In some embodiments, a database of features describing the molecular, phenotype, or population signals 1318 of molecular variants (testing) 1316 can be provided to the generated Functional Models (m_{F}) to calculate and create a database of functional scores 1324 describing the predicted phenotypic impact 1322 of molecular variants (testing) 1316. As would be appreciated by a person of ordinary skill in the art, the performance (e.g. accuracy) of the predicted phenotypic impacts 1322 (e.g., functional score 1324) of molecular variants can be determined against known phenotypic impacts of molecular variants, such as testing molecular variants 1316. As would be appreciated by a person of ordinary skill in the art, the Functional Models (m_{F}) can be applied to pre-compute, or compute on demand, the functional scores of molecular variants not included in training, validation, or testing phases within a testing layer 1314. In some embodiments, such scoring and evaluation can occur in a functional scoring and classification layer 1326 to, for example, examine the phenotype impact classification accuracy permitted on the basis of functional scores 1324.

In some embodiments, additional annotation features 1306, 1320 may be provided during training and testing (prediction generation) of Functional Models (m_{F}). In some embodiments, the annotation features 1306 and 1320 may encompass a plurality of independent (e.g., non-assayed) features (e.g., evolutionary, population, functional (e.g., annotation-based), structural, dynamical, and physicochemical features associated with variants, genomic coordinates, transcript (e.g., RNA) coordinates, translated (e.g., protein) coordinates, amino acids, and various others as would be appreciated by a person of ordinary skill in the art), describing changes associated with the changes in genotype (e.g., sequence, molecular variants).

As would be appreciated by a person of ordinary skill in the art, a diverse array of sources for phenotypic impacts (e.g., labels) of molecular variants can be used to define Truth Sets, including (e.g., public and or private) clinical and non-clinical variant databases (e.g., ClinVar, HumVar, VariBench, SwissVar, PhenCode, PharmGKB, or locus-specific databases), and outcome databases.

In some other embodiments, the present disclosure provides systems and methods for deriving functional scores and functional classifications via statistical (e.g., machine) learning to generate a Functional Model (m_{F}) that associates molecular, phenotype, or population signals (e.g., features)—derived from one or more molecular measurements, molecular processes, molecular features, and/or molecular scores—with phenotypic impacts (e.g., labels) of molecular variants computed directly from distinct molecular, phenotype, or population signals, via regression and classification techniques. In some embodiments, this approach may permit, for example, deriving functional scores and functional classifications that predict the relative mutation burden, mutation rate, or mutation signatures of samples from subjects harboring specific molecular variants. In some embodiments, functional scores or functional classifications from such assays may permit informing on the lifetime risk of developing cancer in test subjects.

As would be appreciated by a person of ordinary skill in the art, regression and classification to generate Functional Models (m_{F}'s) may rely on various statistical (e.g., machine) learning techniques for semi-supervised or supervised learning, including, but not limited to, Random Forests (RFs), Gradient Boosted Trees (GBTs), Zero Rules (ZRs), Naive Bayesian (NBs), Simple Logistic Regression (LRs), Support Vector Machines (SVMs), k-Nearest Neighbors (kNNs), and approaches deploying a wide-array of Artificial Neural Network (ANN) architectures and techniques. In some embodiments, the present disclosure describes the use of molecular state-specific, molecular signals for the derivation of molecular state-specific functional scores or functional classifications. In some other embodiments, the present disclosure describes the use of multi-state matrices of molecular signals for the derivation of molecular state-aware functional scores or functional classifications. In some embodiments, the present disclosure describes the use of Convolutional Neural Networks (CNNs) to learn patterned-associations between functional scores or functional classifications and molecular signals distributed across molecular states.

FIG. 1A illustrates the application of DML processes and systems in genes of the RAS/MAPK pathway, according to some embodiments. The RAS/mitogen-activated protein kinase (MAPK) pathway can play a role in cellular proliferation, differentiation, survival and death, and somatic mutations in RAS/MAPK genes can have a role in the development, progression, and therapeutic response of diverse cancer types through the activation and disregulation of MAPK/ERK signaling. In addition, inherited (e.g., germline) mutations in RAS/MAPK genes have been associated with multiple autosomal dominant congenital syndromes, including but not limited to Noonan syndrome (NS), Costello syndrome (CS), and cardio-facio-cutaneous (CFC) syndrome, and LEOPARD syndrome (LS), which present in patients with characteristic facial appearances, heart defects, musculocutaneous abnormalities, and mental retardation, as well as abnormalities of the skin, inner ears and genitalia (Aoki et al. 2008). For example, mutations in the protein tyrosine phosphatase, non-receptor type 11 (PTPN11) and the dual specificity mitogen-activated protein kinase kinase 1/2 genes (MAP2K1, MAP2K2) have been recurrently observed in Noonan and CFC patients, with PTPN11 mutations present in as many as 50% of Noonan patients (Aoki et al. 2008).

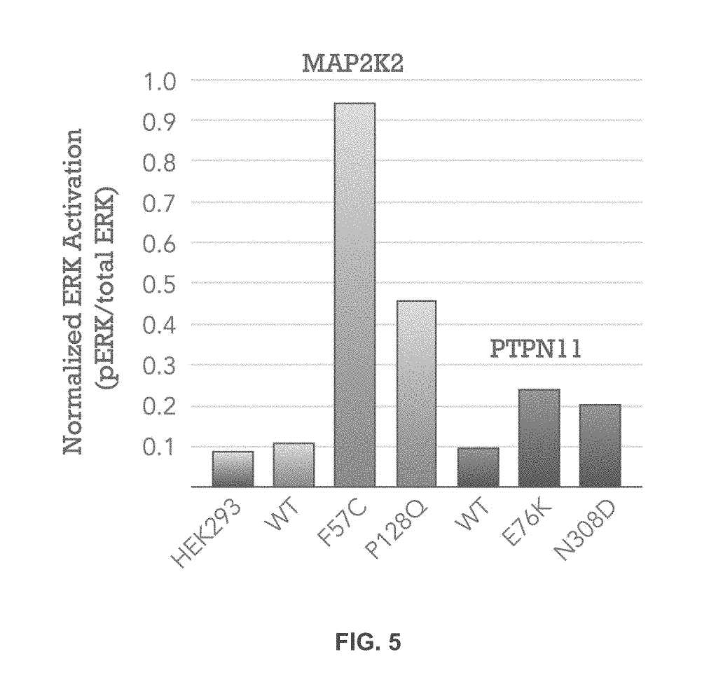

Embodiments can use wildtype, somatic, and germline molecular variants of key RAS/MAPK pathway constituents, such as HRAS (e.g., G12V), PTPN11 (e.g., E76K and N308D), and MAP2K2 (e.g., F57C and P128Q), that are constructed and overexpressed in HEK293 cells. Embodiments can select cells with 1 mg/ml puromycin to ensure expression of the exogenously introduced functional elements (e.g., genes), and RAS/MAPK pathway activation can be verified using an enzyme-linked immunosorbent assays (ELISA) for phospho-ERK protein and total ERK protein abundances (see FIG. 5). To generate single-cell RNA-seq data, embodiments can target for capture 500 cells for each molecular variant using a 10× Genomics Chromium system. Capture and subsequent single-cell library generation can be performed according to manufacturer's recommendations. The resultant libraries for each functional element (e.g., gene) can be pooled and sequenced on an Illumina MiniSeq sequencer until the average reads per cell for each genotype exceeds 30,000 reads/cell. Single-cell RNA-seq processing (e.g., single cell quality control, normalizations, transcriptome counts, etc.) can be performed using the 10× Genomics Cell Ranger 2.1.0 pipeline and default settings.

FIGS. 1B and 1C, illustrate the projection of mammalian cells (e.g., HEK293) harboring wildtype and mutant PTPN11 and MAP2K2, for molecular variants associated with germline disorders (F57C, P128Q, and N308D) as well as somatic disorders (E76K), according to some embodiments. Cells can be projected on a two-dimensional plane derived by t-Stochastic Neighbor Embedding (tSNE) on the basis of molecular scores (e.g., lower-order) determined from scaled, normalized unique molecular identifier (UMI) counts of single-cell gene expression, according to some embodiments. For each gene, tSNE projections are shown based on higher-order molecular scores derived via application of broad, generalized algorithms standard in the field (e.g., Principal Component Analysis, PCA) and custom-developed solutions, including cell-type, gene- or pathway-specific Autoencoders (AE) trained for robust, compressed representation of lower-order molecular scores. In some embodiments, the Autoencoder can be constructed as a neural network with fully connected layers, containing symmetric numbers of neurons (e.g., across layers) around the middle layer, and with rectified linear-units (ReLu) for activation. In some embodiments, the Autoencoder can be trained using an Adam optimizer and optimized against a mean-squared error (MSE) loss function.

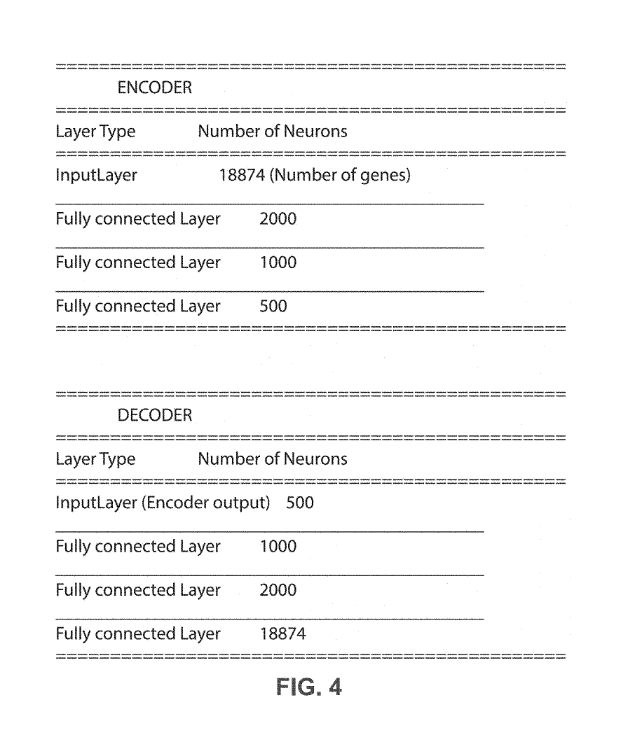

As illustrated in FIGS. 1B and 1C, cellular projections from customized, cell-type and pathway-specific Autoencoders (AEs) can improve the hyperdimensional separation between model systems (e.g., cells) harboring neutral (e.g., wildtype) and disease-associated molecular variants (e.g., N308D, E76K), relative to generalized dimensionality reduction algorithms. A Denoising Autoencoder (AE) was trained on 8.3 Million lower-order molecular scores from greater than 18,800 genes detected in 3,495 single HEK293 cells harboring wildtype and mutant versions of RAS/MAPK genes. Training was performed in 30 epochs with a mini-batch size of 10, with noise simulations following a randomized 5% reduction in the sampling of UMI counts between epochs. The architecture of the utilized fully-connected, symmetric Autoencoder is shown in FIG. 4. Whereas conventional approaches in the domain for the scaling, normalization, and dimensionality reduction of lower-order molecular scores can fail to separate the tSNE-projections of cells harboring Noonan syndrome (NS; N308D) molecular variants and wildtype PTPN11, customized cell-type and pathway-specific Autoencoders can show a robust separation of cells harboring somatic (E76K) and germline (N308D) disorder molecular variants from wildtype cells in PTPN11.

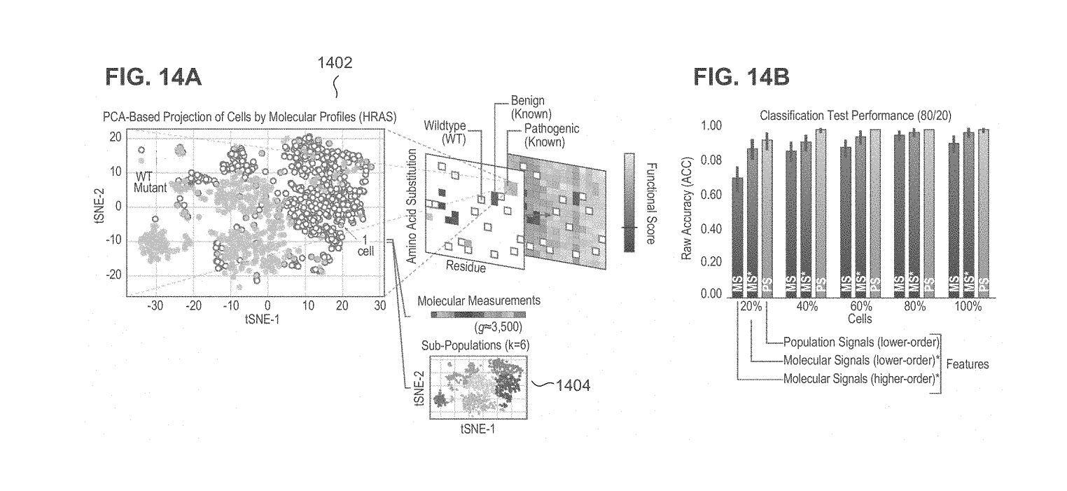

According to some embodiments, FIGS. 14A and 14B illustrates the performance of systems and methods for the binomial classification of molecular variants with two distinct phenotypic impacts as determined in mammalian cells harboring either disease-associated (e.g., pathogenic) genotypic (e.g., sequence) variants (e.g., G12V) and a wild-type (e.g., benign) genotypic (e.g., sequence) version of the human HRAS gene, or a third member of the RAS/MAPK pathway which encodes the onco-protein h-Ras (also known as transforming protein p21). A small G protein in the Ras subfamily of the Ras superfamily of small GTPases, h-Ras—once bound to guanosine triphosphate—can activate RAF-family kinases (e.g., c-Raf), leading to cellular activation of the MAPK/ERK pathway.

FIG. 14A illustrates the projection 1402 of wildtype and mutant mammalian cells (HEK293) on the two-dimensional plane derived by t-Stochastic Neighbor Embedding (tSNE) of cells on the basis of their normalized, single-cell gene expression measurements. As indicated in FIG. 14A, lower-order molecular scores can be derived from the molecular measurements of greater than 33,500 genes, with an average of ˜3,500 molecular measurements made per cell. Principal Component Analysis (PCA) can be applied to derive higher-order molecular scores that reduce the dimensionality of the lower-order molecular scores. Gaussian Mixture Models (GMMs) can be applied to assign the projected cells to molecular states 1404, defining, for example, N=6 sub-populations of cells on the basis of the lower-order molecular scores derived from their normalized, single-cell gene expression measurements (e.g., UMI counts). Pseudo disease-associated genotypes and benign genotypes can be generated by randomly assigning mutant and wildtype cells to, for example, k_{P}=15 disease-associated and k_{B}=15 benign pseudo-populations, respectively. To train and test a machine learning Functional Model (m_{F}) capable of discriminating between disease-associated and benign genotypes, pseudo-populations (k_{P}1-15, k_{B}1-15) can be divided into training and testing sets applying, for example, an 80/20 cross-validation scheme, resulting in, for example, k_{TRAIN}=12 training and k_{TEST}=3 testing genotypes of each class label (e.g., disease-associated and benign), collectively termed a Truth Set. This procedure can be repeated, for example, 1=25 iterations in each of f=5 folds, wherein within each fold the cells within the pseudo-population (e.g., k_{P}1-15, k_{B}1-15) can be sampled with replacement to retain, for example, 20%, 40%, 60%, 80%, or 100% of the cells. In each iteration, fold, and sampling, lower-order molecular signals and higher-order molecular signals for disease-associated and benign genotypes can be computed as the mean of the lower-order molecular scores and higher-order scores, respectively. In each iteration, fold, and sampling, population signals for disease-associated and benign genotypes can be determined as the fraction of cells corresponding to each of the, for example, N=6 sub-populations. In each iteration, fold, and sampling, a machine learning Functional Model (m_{F}) can partition disease-associated and benign genotypes from the Truth Set on the basis of the lower-order molecular signals, higher-order molecular signals, or population signals observed in the k_{TRAIN} data. This Functional Model (m_{F}) can be trained utilizing a 10× cross-validation strategy as well as a Random Forest estimator to partition variants. In each iteration, fold, and sampling, the trained Functional Model (m_{F}) can predict the class label (e.g., disease-associated or benign) of the k_{TEST} pseudo-populations on the basis of their lower-order molecular signals, higher-order molecular signals, or population signals. As illustrated in FIG. 14B, this approach can result in robust discrimination between disease-associated and benign genotypes on the basis of the lower-order molecular signals, higher-order molecular signals, and population signals determined within populations of mutant and wildtype cells.

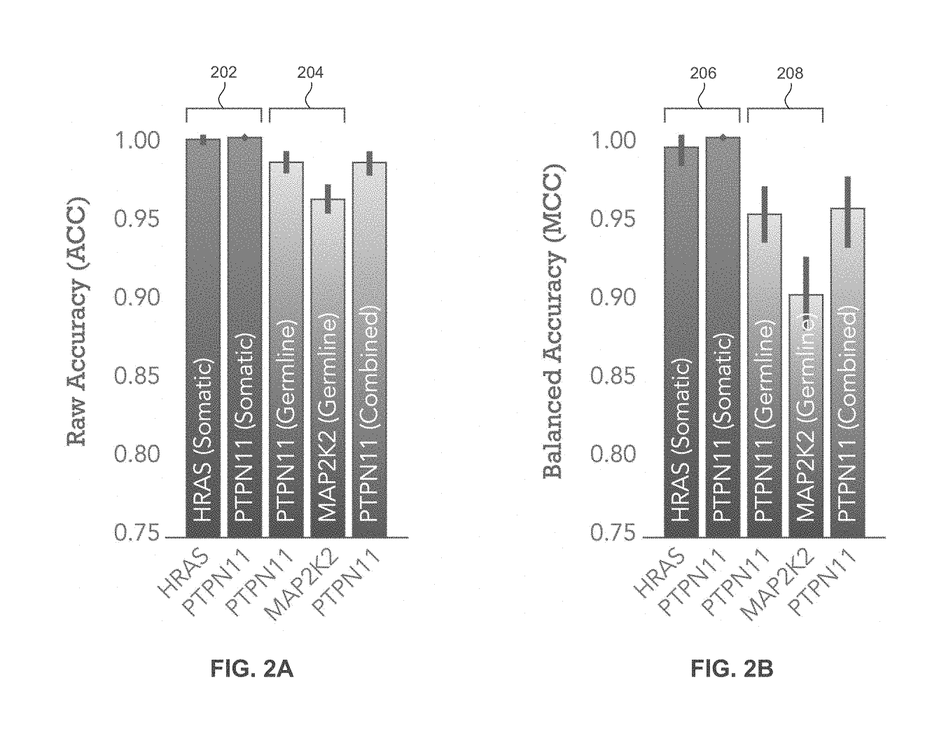

To evaluate the performance of DML processes and systems as a scalable solution for the accurate identification of disease-associated (e.g., pathogenic) molecular variants across multiple genes and disorders, a uniform, distributed DML processing pipeline can be deployed for the pre-processing, scaling, normalization, dimensionality reduction, and computation of molecular and population signals on, for example, three genes of the RAS/MAPK pathway, HRAS, PTPN11, and MAP2K2. Applying a similar training/testing schema for the evaluation of classification accuracies as above, the DML processes can achieve (e.g., median) raw classification accuracies 202 of ˜99.9% and ˜100% in the analysis of somatic cancer-driving molecular variants in HRAS (e.g., G12V) and PTPN11 (e.g., E76K), respectively, and (e.g., median) raw classification accuracies 204 of ˜98.5% and ˜96.1% in the analysis of molecular variants form germline (e.g., inherited) disorders in PTPN11 (e.g., N308D) and MAP2K2 (e.g., F57C, P128Q), respectively, as demonstrated in FIG. 2A. The balanced accuracies 206, 208 (e.g., Matthews Correlation Coefficient, MCC) in the classification of molecular variants known to cause somatic disorders in HRAS, somatic disorders in PTPN11, germline disorders in PTPN11, and germline disorders in MAP2K2, can be ˜99.4%, ˜100%, ˜95.2%, and ˜90.1%, respectively, as shown in FIG. 2B. The raw classification accuracies (e.g., ACC) and balanced classification accuracies (e.g., MCC) in the analysis of disease-associated (e.g., somatic and germline, combined) molecular variants can be ˜98.4% and ˜95.6%, respectively, on the basis of the herein described molecular and population signals.

In some embodiments, the present disclosure provides systems and methods for the derivation of model system-level (e.g., cell-level) phenotypic scores through application of statistical machine learning models to associate lower-order and higher-order molecular scores with the known phenotypic impacts of variants harbored within model systems (e.g., cells). FIGS. 3A and 3B illustrates the cell-level raw classification accuracy of machine learning models trained to derive phenotypic scores in cells harboring wildtype and mutant versions of MAP2K2, according to some embodiments.

In FIG. 3A, germline and enhanced bars can indicate the average classification accuracy of test cells harboring MAP2K2 germline-disorder molecular variants excluded from training, on the basis of cell phenotype scores, where training was exclusively based on MAP2K2 neutral and germline-disorder molecular variants (e.g., germline 302) or included data from PTPN11 germline-disorder molecular variants (e.g., enhanced 304). Germline 302 and enhanced 304 bars in FIG. 3B indicate the average classification accuracy of test MAP2K2 germline-disorder molecular variants excluded from training, as determined on the basis of the predominant cell phenotype scores for populations of cells with varying numbers of cells. As in FIG. 3A, germline and enhanced bars can correspond to the raw accuracies in classification of test molecular variants where training was exclusively based on MAP2K2 neutral and germline-disorder molecular variants (e.g., germline) or included data from PTPN11 germline-disorder molecular (e.g., enhanced).

FIGS. 3A and 3B illustrates data obtained with a logistic regression (LR) classifier trained for binary classification of cells harboring disease-associated molecular variants and cells harboring wildtype MAP2K2, on the basis of higher-order molecular scores computed as the top 100 principal components from (e.g., scaled and or normalized) lower-order molecular scores. Sets of cells for training and testing can be created by partitioning molecular variants into training and testing bins, and partitioning cells into corresponding training and testing sets on the molecular variant genotypes, such that specific sets of cells with specific disease-associated molecular variant are excluded from training. As such, classification test performance can be computed on complete populations of cells harboring variants excluded from training. As shown in FIGS. 3A and 3B, the average per-cell classification accuracy across molecular variants associated with germline (e.g., inherited) disorders in MAP2K2 can be ˜80.3%.

In some embodiments, the present disclosure describes the learning and prediction of the phenotypic consequences of molecular variants on the basis of molecular, phenotype, or population signals assayed in multiple genes, molecular elements, within the same, related, or interacting pathways. As shown in FIGS. 3A and 3B, inclusion of data from PTPN11 molecular variants associated with germline (e.g., inherited) disorders can increase the average per-cell classification accuracy across germline-disorder molecular variants in MAP2K2 from ˜80.3% (e.g., germline 302) to ˜92.8% (e.g., enhanced 304), thereby demonstrating the ability of the disclosed DML processes and systems to identify and leverage coherent cellular properties for accurate classification of the phenotypic impacts of molecular variants across multiple functional elements. As shown in FIGS. 3A and 3B, the increased performance in per-cell classification can result in increases in classification of molecular variants on the basis of the majority-type classification from populations of cells harboring molecular variants.

In some embodiments, the present disclosure provides systems and methods for deriving functional scores and functional classifications for individual functional elements (e.g., individual genes). In some embodiments, the present disclosure provides methods for deriving functional scores and functional classifications across a multitude of functional elements leveraging concordant molecular signals across molecular variants within a plurality of functional elements. In some embodiments, the present disclosure describes systems and methods combining the use of mutagenesis, molecular barcoding, molecular cloning, and cellular pooling techniques to generate populations of cells in which molecular variants in distinct functional elements are uniquely created, barcoded, or both.

In some embodiments, independent or disjoint estimates of molecular, phenotype, or population signals (e.g., features) may be used to derive independent or disjoint functional scores and functional classifications via statistical (e.g., machine) learning to associate molecular signals (e.g., features) with phenotypic impacts (e.g., labels) of molecular variants via regression and classification techniques, respectively.

In some embodiments, feature weights from statistical (e.g., machine) learning models generated using independent or disjoint estimates of each molecular, phenotype, or population signal are computed, collected and utilized for robust feature selection using techniques as would be appreciated by a person of ordinary skill in the art. In some embodiments, the present disclosure provides methods for deriving functional scores and functional classifications via statistical (e.g., machine) learning to associate the identified robust molecular, phenotype, or population signals (e.g., robust features) with phenotypic impacts (e.g., labels) of molecular variants via regression and classification techniques, respectively.

In some embodiments, the present disclosure describes systems and methods for deriving functional scores and functional classifications from a plurality of statistical (e.g., machine) learning models generated using independent or disjoint estimates of molecular signals, applying either model selection or model combination (e.g., mixing) techniques (Pan et al. 2006).

In some embodiments applying model selection techniques, a model selection criterion measuring the predictive performance of a model or the probability of it being the true model may be used to compare the models and selection can be applied to maximize an estimate of the selection criterion. As would be appreciated by a person of ordinary skill in the art, a diversity of model selection criteria can be applied, including (but not limited to) the Akaike Information Criterion (AIC), Bayesian Information Criterion (BIC), Cross-Validation (CV), Bootstrap (Efron 1983; Efron 1986; Efron and Tibshirani 1997), or adaptive model selection criteria (George and Foster 2000; Shen and Ye 2002; Shen et al. 2004) computed on the training data or input test data, as exemplified by test input-dependent weights (IDWs). The IDW for a candidate model may be defined as the probability of the model giving a correct prediction for a given input or a reasonable measure to quantify the predictive performance of the model for the input test data (Pan et al. 2006).

In some other embodiments applying model combination techniques, a combined model can be generated by applying ensemble methods, by taking an equally or unequally weighted average of the outputs from individual models (Ripley 2008; Hastie et al. 2001). For example, ensemble methods can include but are not limited to Bayesian model averaging, stacking, bagging, random forests, boosting, ARM, and using performance metrics (e.g., AIC and BIC) as weights computed on training data (Burnham and Anderson 2003; Hastie et al. 2001) or computed on input test data (Pan et al. 2006). In some other embodiments applying model combination techniques, a combined model can be generated applying an Artificial Neural Network (ANN) architecture. In some embodiments, the present disclosure describes systems and methods for deriving functional scores and functional classifications from a plurality of statistical (e.g., machine) learning models generated using independent or disjoint estimates of molecular signals that involve applying various noise-control techniques (e.g., a Bootstrap Ensemble with Noise Algorithm (Yuval Raviv 1996)).

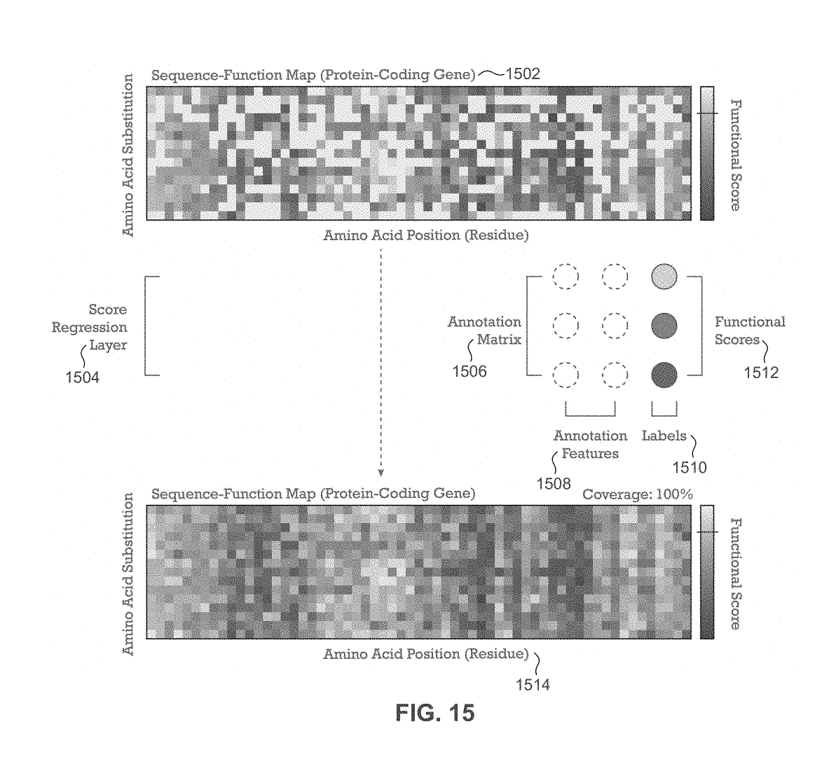

In some embodiments, the present disclosure describes systems and methods for estimating functional scores and functional classifications for molecular variants applying statistical (e.g., machine) learning techniques to generate an Inference Model (m_{I}) that models the relationship between (e.g., assay end-points) functional scores or functional classifications and a plurality of dependent (e.g., assayed) features (e.g., molecular, phenotype, or population signals) or independent (e.g., non-assay) features (e.g., evolutionary, population, functional (e.g., annotation-based), structural, dynamical, and physicochemical features associated with variants, genomic coordinates, transcript (e.g., RNA) coordinates, translated (e.g., protein) coordinates, amino acids, and various others as would be appreciated by a person of ordinary skill in the art). As would be appreciate by a person of ordinary skill in the art, such Inference Model (m_{I}) may permit estimating functional scores and functional classifications for molecular variants with or without the explicit use of molecular, phenotype, or population signals, molecular measurements, molecular processes, molecular features, or molecular scores. In some embodiments, such methods may permit inferring sequence-function maps describing functional scores and functional classifications for molecular variants beyond those for which the functional scores and functional classifications were directly assayed. In some embodiments, as illustrated in FIG. 15, such systems and methods may permit inferring a sequence-function map 1514 describing the functional scores or functional classifications for all possible non-synonymous variants in a protein coding gene using functional scores and functional classifications from a sequence function map 1502, representing a subset of the possible non-synonymous variants. In some embodiments, this inference can utilize a score regression layer 1504 that accesses an annotation matrix 1506, consisting of annotation features 1508, labels 1510, and functional scores 1512 as inputs. As would be appreciated by a person of ordinary skill in the art, a multiplicity of statistical validation and cross-validation techniques can be applied to monitor and ensure the accuracy of estimated functional scores and functional classifications.

In some embodiments, and as illustrated in FIG. 16, the present disclosure describes systems and methods for determining the phenotypic impact (e.g., pathogenicity, functionality, or relative effect) of molecular variants through a series of modeling layers that (a) collect or generate existing knowledge or reliable predictions of the phenotypic impacts of molecular variants, (b) enlarge the set of molecular variants with known or predicted phenotypic impacts through functional modeling (e.g., performed via a Functional Modeling Engine (FME)) of sampled molecular variants of known, high-confidence predicted, and unknown phenotypic impacts, and (c) further complete the set of molecular variants with known or predicted phenotypic impacts through inference modeling. In combination, these layers can expand (or optimize) the scope of the Truth Sets available for Functional Model (m_{F}) 1607 generation and reduce (or optimize) the required scope of Functional Model (m_{F}) 1607 generated support for Inference Model (m_{I}) 1609 generation. In some embodiments, these systems and methods can overcome limitations in training, validation, and testing for functional elements (e.g., genes) and contexts with limited availability of molecular variants of known phenotypic impact (e.g., pathogenicity, functionality, or relative effect). Such systems and methods thereby enable elucidating the phenotypic impacts of molecular variants for functional elements (e.g., genes) with otherwise limited data for model generation and can reduce overall costs.

In some embodiments, and as illustrated in FIG. 16, such systems and methods may combine one or more of the following modeling layers to achieve this: (1) a Prediction Model (m_{P}) 1603, (2) a Sampling Model (m_{S}) 1605, (3) a Functional Model (m_{F}) 1607, and (4) an Inference Model (m_{I}) 1609. In some embodiments, the present disclosure describes systems and methods that access molecular variants with known phenotypic impacts (e.g., pathogenic or benign) from pre-existing sources to populate a sequence-function map 1602 describing the phenotypic impacts of molecular variants in a gene/functional element. In some embodiments, a well-characterized Prediction Model (m_{P}) 1603 can be used to generate an enhanced sequence-function map 1604, incorporating the phenotypic impacts of molecular variants with high-confidence predictions. In some embodiments, a Sampling Model (m_{S}) 1605 is applied to generate a set of genotypes (e.g. molecular variants) 1606 containing (a) a Truth Set by selecting or sub-sampling molecular variants with known or high-confidence, predicted phenotypic impacts, and (b) a Target Set of molecular variants of unknown phenotypic impacts.

In some embodiments, the present disclosure describes the use of statistical (e.g., machine) learning to generate a Functional Model (m_{F}) 1607 that associates molecular, phenotype, or population signals and functional scores and functional classifications as learned from molecular variants in the Truth Set (e.g., from genotypes 1606) to predict the functional scores and functional classifications of molecular variants in the Target Set (e.g., from genotypes 1606), thereby yielding a sequence-function map of functional scores 1608.

In some embodiments, as illustrated in FIG. 16, the Functional Model (m_{F}) 1607 accesses enhanced Truth Sets 1611 and 1612 that include molecular and population signals from a plurality of functional elements (e.g., genes) in the same, related, or interacting pathways. This capability can allow the system to generate a Functional Model (mF) 1607 for functional elements (e.g., genes) with limited availability—or devoid—of molecular variants with known or high-confidence, predicted phenotypic impacts, on the basis of molecular, phenotype, or population signals from functional elements (e.g., genes) with coherent mechanisms of action. FIGS. 3A and 3B illustrates an example of this.

In some embodiments, the phenotypic impacts of known molecular variants, high-confidence predicted molecular variants, and functionally-modeled molecular variants can be leveraged by an Inference Model (mI) 1609 that models the relationship between phenotypic impacts and a plurality of dependent (e.g., assayed) features (e.g., molecular, phenotype, or population signals) or independent (e.g., non-assay) features (e.g., evolutionary, population, functional (e.g., annotation-based), structural, dynamical, and physicochemical features associated with variants, genomic coordinates, transcript (e.g., RNA) coordinates, translated (e.g., protein) coordinates, amino acids, and various others, as would be appreciated by a person of ordinary skill in the art) to yield an augmented sequence-function of functional scores 1610. As would be appreciate by a person of ordinary skill in the art, such Inference Model (m_{I}) 1609 may permit estimating the phenotypic impacts of molecular variants with or without the explicit use of molecular, phenotype, or population signals.

In some embodiments, the present disclosure describes systems and methods for the optimization of cost-efficiency of molecular variant classification through the staged deployment of Deep Mutational Learning (DML) processes and systems on Truth and Target (Query) Sets of molecular variants. Some embodiments include a Stage I Optimization 610 step as illustrated in, for example, FIG. 6), where model systems (e.g., cells) harboring Truth Set variants are assayed at high model system (e.g., cell) number and read-depth—in Cell Number, Read-Depth Optimization 612—to generate high-quality data for Dimensionality Reduction Model (m_{DR}) 614—such as an Autoencoder (m_{AE})—and Functional Model (m_{F}) 616 optimizations. In this first stage, dimensionality reduction and classification accuracies for the target phenotypic impacts of molecular variants can be optimized to identify combinations of Dimensionality Reduction Models (614), Functional Models (616), and Cell-Numbers, Read-Depths (612) that guarantee robust target performance. In some embodiments, subsampling and noise simulations can be utilized to train and model performance of Dimensionality Reduction Models and Functional Models. As illustrated in FIG. 6, some embodiments include a Stage II Production 620 step, where model systems (e.g., cells) harboring Target Set variants—and, optionally, Truth Set variants can be assayed in deployments with (e.g., optimal or minimal) Cell-Numbers and/or Read-Depths 622 identified as robust when specific Dimensionality Reduction Models 624 and Functional Models 626 are deployed.

In some embodiments, the present disclosure describes systems and methods for determining the phenotypic impact (e.g., pathogenicity, functionality, or relative effect) of molecular variants identified within a biological sample or record of a subject on the basis of the functional scores and functional classifications determined as described above. In some embodiments, time-stamped records of incorporation of functional scores and functional classifications for a set of (e.g., a plurality of unique) molecular variants may be created, evaluated, validated, selected, and applied to determine the phenotypic impact of molecular variants identified within a biological sample or record of a subject.

In some embodiments, the present disclosure describes systems and methods for determining the phenotypic impact (e.g., pathogenicity, functionality, or relative effect) of molecular variants identified within a biological sample or record of a subject on the basis of the predictor scores or predictor classifications from computational predictors generated by applying statistical (e.g., machine) learning methods to leverage the functional scores and functional classifications.

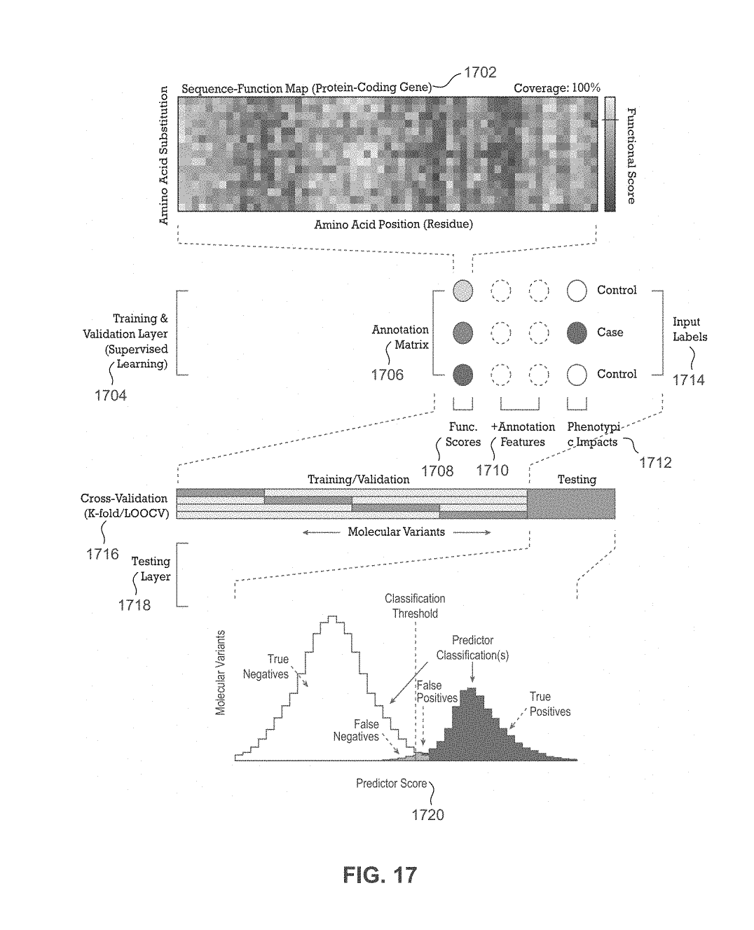

In some embodiments, and as illustrated in FIG. 17, the present disclosure describes methods for generating (e.g., lower-order) Variant Interpretation Engines (VIEs) that can be gene- and condition-specific, through statistical (e.g., machine) learning techniques that model the phenotypic impacts 1712 of molecular variants on the basis of input labels 1714 and an annotation matrix 1706 comprising their functional scores 1702, 1708 (or functional classifications) and other annotation features 1710, including commonly used features in the creation of the computational predictors, including but not limited to evolutionary, population, functional (e.g., annotation-based), structural, dynamical, and physicochemical features associated with variants and residues of functional elements. In some embodiments, the training and validation layer 1704 may employ cross-validation techniques 1716 (e.g., K-fold or LOOCV) to train and quality control VIEs that are subsequently evaluated by a testing layer 1718 to derive predictor scores 1720 used in molecular variant classification.

In some embodiments, the present disclosure further describes systems and methods for generating pathway- and condition-specific (higher-order) Variant Interpretation Engines (VIEs) applying model combination techniques that integrate (lower-order) gene- and condition-specific Variant Interpretation Engines (VIEs) from a plurality of genes in target pathways of interest. In other embodiments, the present disclosure further describes systems and methods for generating pathway- and condition-specific (higher-order) Variant Interpretation Engines (VIEs) through statistical (e.g., machine) learning techniques that model the phenotypic impacts of molecular variants on the basis of their functional scores, functional classifications, and other features commonly used in the creation of the computational predictors, including but not limited to evolutionary, population, functional (annotation-based), structural, dynamical, and physicochemical features associated with variants and residues of functional elements.

In some embodiments, the present disclosure describes systems and methods for determining the phenotypic impact (e.g., pathogenicity, functionality, or relative effect) of molecular variants identified within a biological sample or record thereof of a subject on the basis of the hotspot scores and hotspot classifications from mutational hotspots computed by applying spatial clustering techniques to identify networks of residues with specific phenotypic impacts leveraging the herein-described and enabled functional scores, functional classifications, and molecular signals associated with molecular variants and residues.

In some embodiments, the present disclosure describes systems and methods for deriving a matrix of functional distances between molecular variants or their corresponding residues by (1) computing a distance metric between molecular variants projected in the N-dimensional space (1≤N≤M) defined by a set of M of functional scores, functional classifications, and molecular signals (as described above), where N<M when dimensionality-reduction techniques are applied to reduce the feature-space of molecular variants. As would be appreciated by a person of ordinary skill in the art, various dimensionality-reduction techniques may be applied including but not limited to techniques reliant on linear transformations—as in principal component analysis (PCA)—or non-linear transformations—as in the manifold learning techniques (e.g., t-distributed stochastic neighbor embedding (tSNE) and kernel principal component analysis (kPCA)). As would be appreciated by a person of ordinary skill in the art, various distance metrics can be utilized, including but not limited to, the Euclidean distance, Manhattan distance (e.g., City-Block), Mahalanobis distance, or Chebychev distance, and various others.

In some embodiments, the present disclosure describes systems and methods for the identification of Significantly Mutated Regions (SMRs) and Networks (SMNs) by measuring and scoring the phenotype-associated mutation density (e.g., number of observed phenotype-associated variants per residue) within spatially-proximal residues of functional elements (e.g., protein-coding genes) through the application of spatial clustering techniques across a plurality of spatial distance metrics, including the herein described and enabled functional distances, sequence distances, structure distances, (co)evolutionary distances, and combinations thereof.

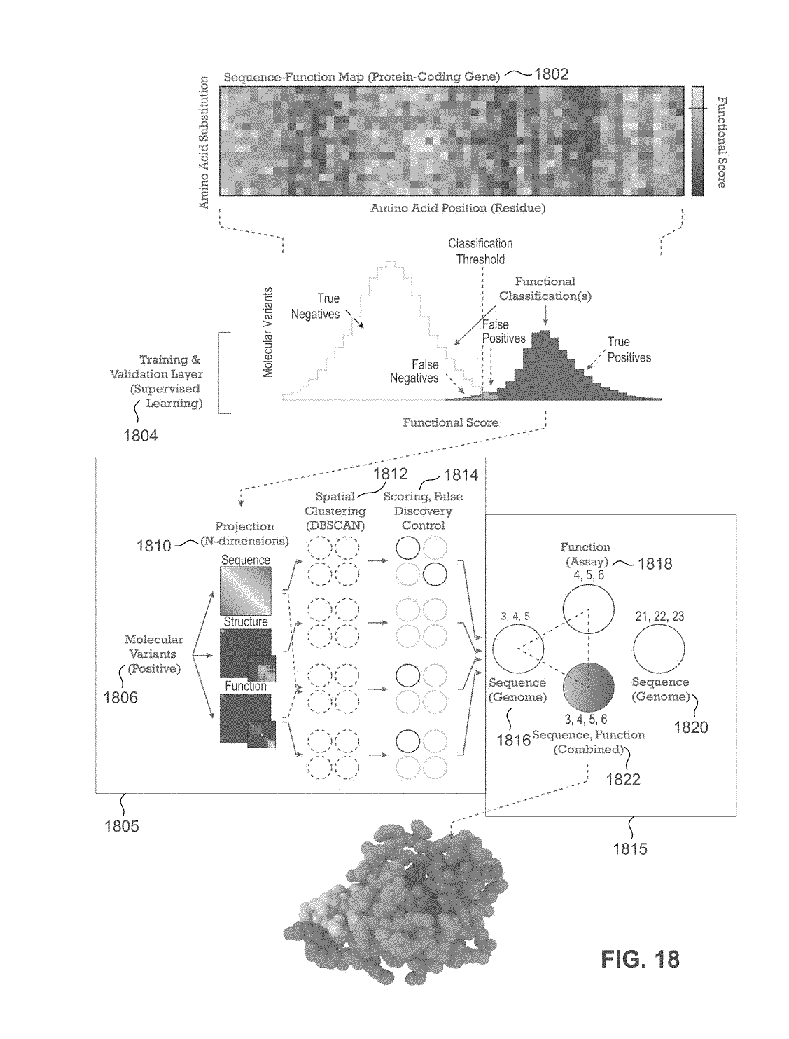

In some embodiments, and as illustrated in FIG. 18, the identification of SMRs/SMNs may apply a Training/Validation Layer 1804 to identify spatial clustering among phenotypically-related or functionally-related molecular variants 1806 as determined on the basis of commonalities in the functional scores of molecular variants. In some embodiments, these commonalities may be identified from the functional scores of molecular variants in a sequence-function map of a protein-coding gene 1802.

In some embodiments, and as illustrated in FIG. 18, the identification of SMRs/SMNs in the Training/Validation Layer 1804 may comprise a series of steps, including but not limited to: (1) SMR/SMN-detection techniques 1805 for the identification of single-residues or networks of residues that are enriched in molecular variants with specific phenotypic associations as have been previously described (Araya et al. 2016, U.S. Patent Application 20160378915A1), and (2) SMR/SMN-selection techniques 1815.

SMR/SMN-detection techniques 1805 can comprise a series of steps including but not limited to: (1.1) projection 1810 of phenotype-associated molecular variants 1806 in functional, sequence, structural, or (co)evolutionary dimensions (or combinations thereof), (1.2) application of spatial clustering techniques 1812 (e.g., DBSCAN) to detect clusters of spatially-proximal phenotype-associated variants, and (1.3) measurement of mutation density, scoring number of phenotype-associated variants per residue in cluster.

SMN-detection techniques 1805 can further comprise the steps denoted in 1814 including, but not limited to: (1.4) scoring of mutation density probability by, for example, computing the (e.g., binomial) probability of obtaining k-or-more (e.g., greater than or equal to k) observed phenotype-associated variants per cluster, given the per-residue mutation rate within each functional element (e.g., protein-coding gene), (1.5) applying multiple hypothesis correction (MHC) across mutation density probabilities of discovered clusters, and (1.6) computing false-discovery rates (FDRs) for the observed (e.g., raw or corrected) mutation density probabilities using background models of mutation density probabilities derived by randomizing positions of the observed phenotype-associated variants within each functional element.

Training/Validation Layer 1804 can further perform the SMR/SMN-selection techniques 1815. SMR/SMN-selection techniques can comprise the steps of (2.1) defining (e.g., raw or corrected) mutation density probabilities and/or false discovery rates (FDRs) as hotspot scores and applying cutoffs to statistically define hotspot classifications, thereby nominating residues in candidate clusters (e.g., sequence 1816, function 1818, and sequence 1820), (2.2) detecting residues in candidate clusters from multiple, distinct projections/spaces, (2.3) assigning residues to individual clusters applying an assignment heuristic (e.g., selecting the cluster largest in size (e.g., cluster with the highest number of residues), and (2.4) identifying SMRs/SMNs as the final set of clusters meeting these criteria. The final set of SMRs/SMNs can be derived from multiple, distinct projections (e.g., sequence 1820, function 1818, or sequence, function (combined) 1822).

In some embodiments, the present disclosure describes systems and methods for the identification of SMRs/SMNs by measuring and scoring the phenotype-associated mutation density (e.g., number of observed phenotype-associated variants per residue) within spatially-proximal residues of functional elements (e.g., protein-coding genes) through the application of spatial clustering techniques across a plurality of spatial distance metrics, where the phenotype-associated variants may be defined on the basis of the functional scores and functional classifications herein described. As would be appreciated by a person of ordinary skill in the art, these methods may allow the determination of clusters of residues in which variants with specifically-defined phenotypic impacts occur.

In some embodiments, the present disclosure describes systems and methods for evaluating the accuracy, performance, or robustness of independent evidence datasets for the interpretation of molecular variants, such as quantitative (e.g., scores) or qualitative (classifications) evidence from computational predictors (e.g., M-CAP, REVEL, SIFT, and PolyPhen2), as well as gene-specific predictors (e.g., PON-P2), mutational hotspots, and population genomics metrics (e.g., allele frequency-based variant classifications), (Amendola et al. 2016) against the herein described functional scores and functional classifications.

In some embodiments, the present disclosure describes systems and methods for computing evaluation metrics to assess concordance between an evidence dataset and the herein described functional scores and functional classifications, and based on these evaluation metrics selecting the best-performing evidence dataset for use in variant interpretation and prioritization. As would be appreciated by a person of ordinary skill in the art, various evaluation metrics can be used to assess the concordance of an evidence dataset against the herein described functional scores or functional classifications. For quantitative evidence (e.g., scores), these may include the Pearson's correlation coefficient, Spearman's rank-order correlation, Kendall correlation, and various others as would be appreciated by a person of ordinary skill in the art. For qualitative evidence (e.g., classifications), these may include accuracy, Matthew's correlation coefficient, Cohen's kappa coefficient, Youden's index (e.g., informedness), F-measure (e.g., F₁ score), true positive rate (e.g., sensitivity or recall), true negative rate (e.g., specificity), positive predictive value (e.g., precision), negative predictive value, positive likelihood ratio, negative likelihood ratio, and diagnostic odds ratio, and various others as would be appreciated by a person of ordinary skill in the art.

In some embodiments, the present disclosure describes systems and methods that may continuously evaluate, validate, and optimize (e.g., select, remove, or modify) diverse evidence datasets on the basis of the above described evaluation metrics, and distribute the best-performing (e.g., independent) evidence datasets to client systems via an Application Program Interface (API) for use in variant interpretation and prioritization practices determining the phenotypic impact (e.g., pathogenicity, functionality, or relative effect) of molecular variants identified within a biological sample or record thereof of a subject.

In some embodiments, the present disclosure describes systems and methods for determining the degree of ascertainment bias, reporting bias, or outcome bias present within a dataset of variants, including clinical datasets (e.g., ClinVar, HumVar, VariBench, SwissVar, PhenCode, or locus-specific databases), population datasets (e.g., ExAC, GnomAD, and 1000 Genomes), or independent evidence datasets for the interpretation of molecular variants, such as but not limited to computational predictors (e.g., M-CAP, REVEL, SIFT, PolyPhen2, and PON-P2). In some embodiments, the present disclosure describes systems and methods for determining biases on the basis of the expected distributions of the herein described functional scores, functional classifications, and molecular signals associated with molecular variants and residues.

In some embodiments, the present disclosure describes systems and methods for the evaluation of a target variant dataset by measuring and scoring the difference between the distributions of functional scores, functional classifications, and molecular signals of molecular variants and residues within the target dataset against the expected distributions of functional scores, functional classifications, and molecular signals of molecular variants from a reference dataset. In some embodiments, the measurement of inherent biases within a target variant dataset may comprise a series of steps, including but not limited to: (1) collection of functional scores, functional classifications, and molecular signals associated with molecular variants in the target and reference datasets, (2) estimating the probability density function of functional scores, functional classifications, or molecular signals associated with molecular variants within the reference dataset, (3) estimating the probability density function of functional scores, functional classifications, or molecular signals associated with molecular variants within the target dataset, and (4) measuring the statistical distance between the target dataset-derived probability density function and the reference dataset-derived probability density function of functional scores, functional classifications, or molecular signals. In some embodiments, the measurement of inherent biases within a target variant dataset comprises a series of steps, including: (5) sampling variants from the reference dataset (e.g., to match the sample population size of the target dataset), (6) estimating the probability density function of functional scores, functional classifications, or molecular signals of the sampled reference dataset in step 5, (7) measuring the statistical distance between the target dataset-derived probability density function and the sampled reference dataset-derived probability density function of functional scores, functional classifications, or molecular signals, (8) iterating steps 5-8 to obtain a robust estimate and confidence intervals of the statistical distance between the probability density function of functional scores, functional classifications, or molecular signals of the target and reference datasets. In some embodiments, the above systems and methods for the detection and statistical evaluation of bias permit the identification of clinical datasets, population datasets, or evidence datasets in which the contained variants have different functional scores, functional classifications, or molecular signals from that expected in a reference dataset.

In some other embodiments, the present disclosure describes systems and methods for evaluating underlying biases within evidence datasets by a series of steps, including but not limited to: (1) partitioning evidence and reference datasets into matching sets of quantiles (e.g., for quantitative evidence scores) or classes (e.g., qualitative evidence classifications); (2) scoring variants within each set (e.g., evidence vs. reference) across a plurality of properties (e.g., evolutionary, population, functional (e.g., annotation-based), structural, dynamical, and physicochemical features associated with variants); (3) estimating the probability density function of each property score within each set (e.g., evidence vs. reference); (4) measuring the statistical distance between the evidence set-derived probability density function and the reference set-derived probability density function of each property score; and (5) identifying properties with statistically significant differences in scores between reference and evidence sets.

In some embodiments, the present disclosure describes systems and methods that may continuously evaluate and select diverse evidence datasets on the basis of the above described bias metrics, and distribute the least-biased (e.g., independent) evidence datasets to client systems via an Application Program Interface (API) for use in variant interpretation and prioritization practices determining the phenotypic impact (e.g., pathogenicity, functionality, or relative effect) of molecular variants identified within a biological sample or record thereof of a subject.

In some embodiments, the present disclosure describes systems and methods for determining the phenotypic impact (e.g., pathogenicity, functionality, or relative effect) of molecular variants identified within a biological sample or record of a subject on the basis of herein described functional scores, functional classifications, predictor scores, predictor classifications, hotspot scores, and hotspot classifications, in functional elements (e.g., genes) and pathways associated with Mendelian disorders (e.g., Table 1), that are known cancer-drivers (e.g., Table 2), pharmacogenomic genes in which genotypic (e.g., sequence) variation is associated with variation in drug response (Table 3), or other clinically-valuable genes (e.g., Table 4).

In some embodiments, the present disclosure describes systems and methods for evaluating, selecting, distributing and utilizing independent evidence—determined to be the best-performing and least biased on the basis of the herein described functional scores and classifications—for the interpretation and prioritization of variants in functional elements (e.g., genes) and pathways associated with Mendelian disorders (e.g., Table 1), that are known cancer-drivers (e.g., Table 2), pharmacogenomic genes in which genotypic (e.g., sequence) variation is associated with variation in drug response (e.g., Table 3), or other clinically-valuable genes (e.g., Table 4).

As discussed above, Table 1 is an example table of functional elements and pathways associated with Mendelian disorders, according to some embodiments. Table 2 is an example table of functional elements and pathways that are known cancer-drivers, according to some embodiments. Table 3 is an example table of pharmacogenomic genes in which genotypic (e.g., sequence) variation is associated with variation in drug response, according to some embodiments. Table 4 is an example table of other clinically-valuable genes, according to some embodiments. Tables 1-4 may be found on page 49 of the specification.

In some embodiments, the present disclosure describes systems and methods for determining the phenotypic impact (e.g., pathogenicity, functionality, or relative effect) of molecular variants identified within a biological sample or record of a subject on the basis of herein described and enabled functional scores, functional classifications, predictor scores, predictor classifications of variants within known targets of pathogenic variation, including (but not limited) to mutational hotspots, or for variants within, for example, 50, 100, 500, and 1,000 base pair (bp) of such hotspots. In some embodiments, the present disclosure describes systems and methods for determining the phenotypic impact (e.g., pathogenicity, functionality, or relative effect) of molecular variants identified within a biological sample or record of a subject on the basis of functional scores, functional classifications, predictor scores, or predictor classifications of variants within regions of constrained variation in a population, or for variants within, for example, 50, 100, 500, and 1,000 bp of such regions. As would be appreciated by a person of ordinary skill in the art, a variety of methods for determining mutational hotspots and regions of constrained variation can be applied.

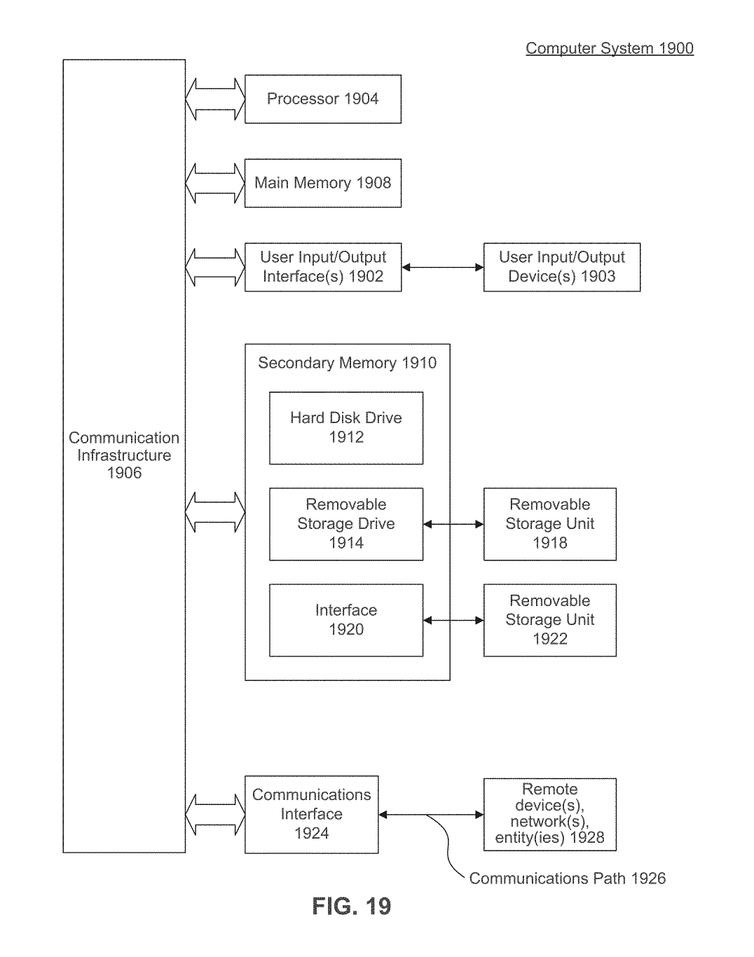

Various embodiments can be implemented, for example, using one or more computer systems, such as computer system 1900 shown in FIG. 19. Computer system 1900 can be used, for example, to implement methods of FIGS. 1A, 6-13, and 15-18. Computer system 1900 can be any computer capable of performing the functions described herein.

Computer system 1900 can be any well-known computer capable of performing the functions described herein.

Computer system 1900 includes one or more processors (also called central processing units, or CPUs), such as a processor 1904. Processor 1904 is connected to a communication infrastructure or bus 1906.

One or more processors 1904 may each be a graphics processing unit (GPU). In an embodiment, a GPU is a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 1900 also includes user input/output device(s) 1903, such as monitors, keyboards, pointing devices, etc., that communicate with communication infrastructure 1906 through user input/output interface(s) 1902.

Computer system 1900 also includes a main or primary memory 1908, such as random access memory (RAM). Main memory 1908 may include one or more levels of cache. Main memory 1908 has stored therein control logic (e.g., computer software) and/or data.

Computer system 1900 may also include one or more secondary storage devices or memory 1910. Secondary memory 1910 may include, for example, a local, network, or cloud-accessible hard disk drive 1912 and/or a removable storage device or drive 1914. Removable storage drive 1914 may be a floppy disk drive, a magnetic tape drive, a compact disk drive, an optical storage device, tape backup device, and/or any other storage device/drive.

Removable storage drive 1914 may interact with a removable storage unit 1918. Removable storage unit 1918 includes a computer usable or readable storage device having stored thereon computer software (control logic) and/or data. Removable storage unit 1918 may be a floppy disk, magnetic tape, compact disk, DVD, optical storage disk, and/any other computer data storage device. Removable storage drive 1914 reads from and/or writes to removable storage unit 1918 in a well-known manner.

According to an exemplary embodiment, secondary memory 1910 may include other means, instrumentalities or other approaches for allowing computer programs and/or other instructions and/or data to be accessed by computer system 1900. Such means, instrumentalities or other approaches may include, for example, a removable storage unit 1922 and an interface 1920. Examples of the removable storage unit 1922 and the interface 1920 may include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 1900 may further include a communication or network interface 1924. Communication interface 1924 enables computer system 1900 to communicate and interact with any combination of remote devices, remote networks, remote entities, etc. (individually and collectively referenced by reference number 1928). For example, communication interface 1924 may allow computer system 1900 to communicate with remote devices 1928 over communications path 1926, which may be wired and/or wireless, and which may include any combination of LANs, WANs, the Internet, etc. Control logic and/or data may be transmitted to and from computer system 1900 via communication path 1926.

In an embodiment, a tangible apparatus or article of manufacture comprising a tangible computer useable or readable medium having control logic (software) stored thereon is also referred to herein as a computer program product or program storage device. This includes, but is not limited to, computer system 1900, main memory 1908, secondary memory 1910, and removable storage units 1918 and 1922, as well as tangible articles of manufacture embodying any combination of the foregoing. Such control logic, when executed by one or more data processing devices (such as computer system 1900), causes such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 12. In particular, embodiments can operate with software, hardware, and/or operating system implementations other than those described herein.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

TABLE 1
Mendelian Disorders
Gene (HGNC Symbol)

BRCA1
BRCA2
APOB
LDLR
PCSK9
SCN5A
APC
MLH1
MSH2
MSH6
STK11
MUTYH
MYH7
LMNA
MYBPC3
TNNI3
TNNT2
KCNQ1
KCNH2
SDHB
ACTA2
MYH11
VHL
RET
SDHAF2
SDHC
SDHD
TP53
TSC1
TSC2
NF2
PTEN
RB1
RYR1
GLA
RYR2
TGFBR1
TGFBR2
ACTC1
CACNA1S
COL3A1
DSC2
DSG2
DSP
FBN1
MEN1
MYL2
MYL3
PKP2
PMS2
PRKAG2
SMAD3
TMEM43
TPM1
WT1
BMPR1A
SMAD4
ATP7B
OTC

TABLE 2
Cancer Drivers (CCG La)
Gene (HGNC Symbol)

TP53
PIK3CA
ARID1A
RB1
PTEN
KRAS
BRAF
CDKN2A
NRAS
FBXW7
STAG2
NFE2L2
NF1
IDH1
ATM
PIK3R1
CASP8
HRAS
MLL2
SF3B1
ERBB2
CREBBP
AKT1
HLA-A
CTCF
ERBB3
CTNNB1
RUNX1
MYD88
SMARCA4
EP300
SETD2
SMARCB1
EGFR
TBL1XR1
U2AF1
EZH2
RAC1
MLL3
IL7R
CD79B
POU2AF1
MAP2K1
PTPN11
CCND1
MAP2K4
TCF7L2
KIT
CDK4
FOXA1
TSC1
FAT1
WT1
BCOR
XPO1
PRDM1
KEAP1
NSD1
PPP2R1A
CDKN1B
ASXL1
MET
RPL5
MYCN
TNFRSF14
FLT3
ALK
KDM5C
KDM6A
APC
PBRM1
STK11
RAD21
EZR
SPOP
TET2
PHF6
IRF4
DDX5
CCDC6
HIST1H3B
CARD11
IDH2
MLL
FGFR2
CDK12
ERCC2
B2M
MED12
CEBPA
NOTCH1
BRCA1
MAP3K1
VHL
DNMT3A
FGFR3
NPM1
FAM46C
CBFB
GATA3
MYB
CDH1
BAP1
ELF3
ZNF198
MALT1
WIF1
KDR
SFRS3
MXRA5
SS18
TAL1
RXRA
TCEA1
HEAB
THRAP3
RUNDC2A
SLC44A3
TNF
TAL2
FLJ27352
LAF4
STK19
DDX10
MSI2
NUTM2A
POU5F1
TRIP11
STAT5B
NCOA2
AZGP1
NCOA1
STAT3
NCOA4
OR52N1
CDKN2a(p14)
CEP1
TFPT
SUFU
HOXA13
DDB2
HOXA11
P2RY8
ECT2L
TRD@
IGH@
SMAD4
RBM10
LASP1
ROS1
KMT2D
WASF3
RBM15
PRKAR1A
KCNJ5
ATRX
EPHA2
BIRC3
HNRNPA2B1
OR4A16
NUTM2B
KLF4
MAP2K2
C15orf21
ERG
CD79A
SRGAP3
MLLT3
MTF
MN1
MLLT2
MLLT7
MLLT6
FAS
C15orf55
POU2F2
EIF2S2
MLLT4
EPS15
HERPUD1
TBC1D12
MLLT1
ALO17
CNOT3
FIP1L1
CBL
OLIG2
HOXC13
NT5C2
ABL1
ZNF521
PLAG1
TPM4
LMO1
LMO2
BLM
NTN4
SLC4A5
IRTA1
JAK3
PMS2
ATP1A1
TERT
CDH11
PTCH
DDX3X
HEY1
MORC4
TLX3
PALB2
BCR
BRCA2
MDM4
MDM2
BRD4
TFG
CSF3R
RPL10
PER1
ITPKB
PDSS2
CREB1
AF3p21
TRIM27
WRN
KIF5B
CHD8
RAB40A
GATA1
ATIC
CD1D
SETBP1
CRTC3
TNFRSF17
COL1A1
DUX4
ACVR1B
C16orf75
NIN
ZNF278
MAF
NF2
AKAP9
CCND2
MAX
MECT1
ARHGEF12
SEPT6
CBLB
FACL6
ALKBH6
CHN1
CBFA2T1
IL6ST
TCEB1
MEN1
FBXO11
HIST1H4I
RALGDS
BUB1B
FHIT
CRLF2
RASA1
TLX1
IGK@
SELP
TXNDC8
CACNA1D
GUSB
NUP214
NKX2-1
INPPL1
CBFA2T3
BCLAF1
TSC2
SDH5
CDC73
ZNF384
CDC27
OTUD7A
SIL
RANBP17
NDRG1
SMC3
FH
PAX7
CD273
HLA-B
PHOX2B
CD274
GNAS
GNAQ
PSIP1
ASPSCR1
GPHN
XIRP2
PAX8
MYOCD
FRMD7
RAP1GDS1
PAX3
AJUBA
SLC34A2
HLF
UBR5
REL
RPS2
GNA11
LHFP
TBX3
SMO
RET
PAPD5
RPS15
SS18L1
MYH11
EIF4A2
LCK
XPA
HSPCA
PPARG
CHIC2
HOXC11
H3F3B
JAK2
TFRC
ZNF620
SOX17
MTCP1
JUN
LCTL
TAF15
NONO
SRSF2
CHCHD7
MAML2
PPM1D
DAXX
H3F3A
JAK1
RIT1
CCND3
TRRAP
MED23
IGL@
SPEN
DIAPH1
CMKOR1
ZNF471
STL
POLE
MAP4K3
ING1
FOXO1A
LIFR
CHEK2
LCP1
AKT2
TPR
NFKB2
FOXL2
COL5A1
FEV
HMGA1
BCL3
HMGA2
CARS
PCSK7
ELL
GMPS
LYL1
BMPR1A
TGFBR2
SLC45A3
GRAF
HLXB9
HIST1H1E
DIS3
WWTR1
PDGFRA
PDE4DIP
ARID5B
ALDH2
STX2
SACS
ARNT
GOPC
SOS1
ITK
DICER1
KEL
CIC
RAB5EP
FVT1
PML
ADNP
FANCA
ABL2
C12orf9
BRIP1
MALAT1
FANCD2
PAFAH1B2
MUTYH
POT1
JAZF1
GNPTAB
FGFR1OP
RAD51L1
DNER
ZNF331
CD70
IKZF1
NCOR1
MLF1
MYH9
SYK
HCMOGT-1
FANCE
FANCF
FANCG
TPM3
NUP210L
INTS12
SDHC
RUNXBP2
BTG1
TTLL9
EML4
SDHB
CDK6
PMX1
PDGFRB
FOXO3A
NTRK1
CLTCL1
SH2B3
EBF1
GPC3
FGFR1
ETV6
NR4A3
SBDS
PIM1
ALPK2
PDGFB
CUL4B
YWHAE
ETV1
BCL10
PBX1
IL21R
CREB3L1
ATF1
FANCC
C2orf44
HSPCB
CANT1
PTPRC
WAS
NFIB
CREB3L2
AF1Q
NOTCH2
ABI1
SH3GL1
NBS1
OMD
SUZ12
TRA@
AF5q31
RSBN1L
BCL11B
MSH6
ERCC5
BCL11A
ERCC3
MSH2
NUMA1
KTN1
TFE3
IL2
MYCL1
LPP
HOXA9
RPL22
MSN
EVI1
BCL7A
AXIN1
NBPF1
ZNF9
MLH1
SFRS2
TRIM33
SIRT4
AXIN2
CIITA
ARHGAP35
SET
ELF4
HIP1
MSF
SOX2
FNBP1
CD74
TCL1A
RAF1
MADH4
COPEB
FLI1
CBLC
GATA2
EXT1
EXT2
MICALCL
DDIT3
D10S170
CDKN2C
MYC
GOLGA5
TRIM23
NTRK3
KLK2
SLC1A3
PRF1
ACSL3
NUP98
ELK4
CYLD
TMPRSS2
DDX6
CCNB1IP1
TTL
ZNF750
TIF1
SOCS1
PNUTL1
FOXQ1
ATP2B3
PMS1
FSTL3
PCBP1
KDM5A
ZNF145
PICALM
EWSR1
AF15Q14
BCL6
GNA13
BCL5
BCL9
ANK3
RHEB
BHD
QKI
PPP6C
CALR
PRCC
FCGR2B
BCL2
RPN1
SSX4
MDS2
TPX2
RARA
ZFHX3
TRB@
MDS1
MAFB
SLC26A3
SGK1
SDHD
CDX2
SSX1
ZRANB3
KIAA1549
SSX2
HOOK3
MTOR
SNX25
TCF1
MGA
LRIG3
PRDM16
ELKS
RHOA
ACO1
ELN
VTI1A
BRD3
MLLT10
RNF43
CDKN1A
ARID2
LCX
TFEB
WHSC1L1
ETV5
ETV4
HOXD11
GAS7
ARHH
IPO7
GOT1
SMAD2
WHSC1
TNFAIP3
TCL6
HOXD13
SDC4
PAX5
MPL
MPO
SFPQ
TCF3
NACA
RECQL4
SMC1A
ERCC4
TCF12
KLHL8
DNM2
CLTC
SMARCE1
DEK
XPC
USP6
FUBP1
PCM1
TRAF7
ZRSR2
FUS
FOXP1
FLG
TOP1
MUC1
TCP11L2
COX6C
MYST4
MUC17
CAMTA1
C3orf70
CUX1
CAP2
TRAF3
MKL1
CCNE1
TSHR
AMER1
CCDC120
CHD4
TAP1

TABLE 3
Pharmacogenomics (Pharm)
Gene (HGNC Symbol)

A2M
ABAT
ABCA1
ABCA12
ABCA3
ABCA8
ABCB1
ABCB11
ABCB4
ABCB5
ABCB6
ABCB9
ABCC1
ABCC10
ABCC11
ABCC2
ABCC3
ABCC4
ABCC5
ABCC6
ABCC8
ABCC9
ABCD1
ABCD2
ABCG1
ABCG2
ABCG8
ABL1
ABO
ACBD4
ACE
ACE2
ACHE
ACP5
ACSS2
ACTG1
ACY3
ACYP2
ADA
ADAM12
ADAM33
ADAMTS1
ADAMTS14
ADCK4
ADCY2
ADCY9
ADD1
ADH1A
ADH1B
ADH1C
ADH7
ADIPOQ
ADK
ADM
ADORA1
ADORA2A
ADORA2A-AS1
ADRA1A
ADRA2A
ADRA2B
ADRA2C
ADRB1
ADRB2
ADRB3
ADRBK2
AFAP1L1
AGAP1
AGBL4
AGO1
AGT
AGTR1
AGXT
AHR
AIDA
AK4
AKR1C3
AKR1C4
AKR7A2
AKT1
AKT2
ALDH1A1
ALDH1A2
ALDH2
ALDH3A1
ALDH5A1
ALG10
ALOX12
ALOX15
ALOX5
ALOX5AP
AMHR2
AMPD1
ANGPT2
ANGPTL4
ANKFN1
ANKK1
ANKRD55
ANKS1B
ANXA11
AOX1
APBB1
APEH
APLF
APOA1
APOA4
APOA5
APOB
APOBEC2
APOC1
APOC3
APOE
APOH
AQP2
AQP9
ARAP1
ARAP2
AREG
ARG1
ARHGEF10
ARHGEF4
ARID5B
ARMS2
ARNT
ARNTL
ARRB2
ARVCF
AS3MT
ASIC2
ASPH
ASS1
ATF3
ATG16L1
ATG5
ATIC
ATM
ATP2B1
ATP5E
ATP7A
ATP7B
AXIN2
B4GALT2
BACH1
BAD
BAG6
BAZ2B
BCAP31
BCHE
BCL2
BCL2L11
BCR
BDKRB1
BDKRB2
BDNF
BDNF-AS
BGLAP
BLK
BLMH
BMP5
BMP7
BRAF
BRD2
BTG4
BTRC
C10orf107
C10orf11
C11orf30
C11orf65
C12orf40
C17orf51
C18orf21
C18orf56
C1orf167
C2
C20orf194
C3
C5
C5orf22
C8orf34
C9orf72
CA10
CA12
CACNA1A
CACNA1C
CACNA1E
CACNA1H
CACNA1S
CACNB2
CACNG2
CALU
CAMK1D
CAMK2N1
CAMK4
CAP2
CAPG
CAPN10
CAPZA1
CARD16
CARTPT
CASP1
CASP3
CASP7
CASP9
CASR
CAT
CBR1
CBR3
CBS
CCDC22
CCHCR1
CCL2
CCL21
CCND1
CCNH
CCNY
CCR5
CD14
CD28
CD38
CD3EAP
CD40
CD58
CD69
CD74
CD84
CDA
CDC5L
CDCA3
CDH13
CDH4
CDK1
CDK4
CDK9
CDKAL1
CDKN2B-AS1
CELF4
CELSR2
CEP68
CEP72
CERKL
CERS6
CES1
CES1P1
CES2
CETP
CFAP44
CFB
CFH
CFI
CFLAR
CFTR
CHAT
CHIA
CHIC2
CHL1
CHRM2
CHRM3
CHRM4
CHRNA1
CHRNA3
CHRNA4
CHRNA5
CHRNA7
CHRNB1
CHRNB2
CHRNB3
CHRNB4
CHST13
CHST3
CHUK
CLASP1
CLCN6
CLMN
CLNK
CLOCK
CMPK1
CNKSR3
CNOT1
CNPY4
CNR1
CNTF
CNTN4
CNTN5
CNTNAP2
COL18A1
COL1A1
COL1A2
COL22A1
COL26A1
COLEC10
COMT
COQ2
CPA2
CPS1
CR1
CR1L
CREB1
CRH
CRHR1
CRHR2
CRP
CRTC2
CRY1
CSK
CSMD1
CSMD2
CSMD3
CSNK1E
CSPG4
CSRNP3
CSRP3
CST5
CTH
CTLA4
CTNNA2
CTNNA3
CTNNB1
CUX1
CUX2
CXCL10
CXCL12
CXCL5
CXCL8
CXCR2
CXCR4
CXXC4
CYB5A
CYB5R3
CYBA
CYCSP5
CYP11B2
CYP19A1
CYP1A1
CYP1A2
CYP1B1
CYP24A1
CYP27B1
CYP2A6
CYP2B6
CYP2B7P1
CYP2C18
CYP2C19
CYP2C8
CYP2C9
CYP2D6
CYP2E1
CYP2J2
CYP2R1
CYP39A1
CYP3A
CYP3A4
CYP3A43
CYP3A5
CYP3A7
CYP4A11
CYP4B1
CYP4F11
CYP4F2
CYP51A1
CYP7A1
DAOA
DAPK1
DBH
DCAF4
DCBLD1
DCK
DCP1B
DCTD
DDC
DDHD1
DDRGK1
DDX20
DDX53
DDX58
DEAF1
DGCR5
DGKH
DGKI
DHFR
DHODH
DIAPH3
DIO1
DIO2
DKK1
DLEU7
DLG5
DLGAP1
DMPK
DNAH12
DNAJB13
DNMT3A
DOCK4
DOK5
DOT1L
DPP4
DPYD
DPYS
DRD1
DRD2
DRD3
DRD4
DROSHA
DSCAM
DTNBP1
DUSP1
DUX1
DYNC2H1
E2F7
EBF1
ECT2L
EDN1
EGF
EGFR
EGLN3
EHF
EIF2AK4
EIF3A
EIF4E2
ENG
ENOSF1
EPAS1
EPB41
EPHA5
EPHA6
EPHA8
EPHX1
EPM2A
EPM2AIP1
EPO
ERAP1
ERBB2
ERCC1
ERCC2
ERCC3
ERCC4
ERCC5
ERCC6L2
EREG
ERICH3
ESR1
ESR2
ETS2
EXO1
F11
F12
F13A1
F2
F3
F5
F7
FAAH
FABP1
FABP2
FADS1
FAM19A5
FAM65B
FARS2
FAS
FASLG
FASTKD3
FAT1
FBXL17
FBXL19
FCAR
FCER1A
FCER1G
FCER2
FCGR2A
FCGR2B
FCGR3A
FDPS
FEN1
FGD4
FGF2
FGF5
FGFBP1
FGFBP2
FGFR2
FGFR4
FHIT
FKBP5
FLOT1
FLT1
FLT3
FLT4
FMO1
FMO2
FMO3
FMO5
FNTB
FOLH1
FOLR3
FOXC1
FOXP3
FPGS
FSHR
FSIP1
FSTL5
FTO
FYN
FZD3
FZD4
G6PD
GABRA1
GABRA3
GABRA6
GABRB1
GABRB2
GABRG2
GABRG3
GABRP
GABRQ
GAD2
GADL1
GAL
GALNT14
GALNT18
GALNT2
GALR1
GAPDHP64
GAPVD1
GATA3
GATA4
GATM
GBP6
GCG
GCKR
GCLC
GDNF
GEMIN4
GFRA2
GGCX
GGH
GHSR
GIPR
GJA1
GLCCI1
GLDC
GLP1R
GLRB
GNAS
GNB3
GNMT
GP1BA
GP6
GPR1
GPR83
GPX1
GPX3
GPX5
GRIA1
GRIA3
GRID2
GRIK1
GRIK2
GRIK3
GRIK4
GRIN1
GRIN2A
GRIN2B
GRIN3A
GRK4
GRK5
GRM3
GRM7
GSK3B
GSR
GSTA1
GSTA2
GSTA5
GSTM1
GSTM3
GSTM4
GSTP1
GSTT1
GSTZ1
H19
HAS3
HCG22
HCP5
HDAC1
HES6
HFE
HIF1A
HLA-A
HLA-B
HLA-C
HLA-DOB
HLA-DPA1
HLA-DPB1
HLA-DPB2
HLA-DQA1
HLA-DQB1
HLA-DRA
HLA-DRB1
HLA-DRB3
HLA-DRB5
HLA-E
HLA-G
HMGB1
HMGB2
HMGCR
HNF1A
HNF1B
HNF4A
HNMT
HOMER1
HOTAIR
HOTTIP
HRH1
HRH2
HRH3
HRH4
HS3ST4
HSD11B1
HSD3B1
HSPA1A
HSPA1L
HSPA5
HSPG2
HTR1A
HTR1B
HTR1D
HTR2A
HTR2C
HTR3A
HTR3B
HTR5A
HTR6
HTR7
HTRA1
HUS1
HYKK
IBA57
IDO1
IFIT1
IFNAR1
IFNB1
IFNG
IFNGR1
IFNGR2
IFNL3
IFNL4
IGF1
IGF1R
IGF2BP2
IGF2R
IGFBP3
IGFBP7
IKBKG
IKZF3
IL10
IL11
IL12A
IL12B
IL13
IL16
IL17A
IL17F
IL17RA
IL18
IL1A
IL1B
IL1RN
IL2
IL21R
IL23R
IL27
IL2RA
IL2RB
IL3
IL4
IL4R
IL6
IL6R
IL6ST
IL7R
ILKAP
IMPA2
IMPDH1
IMPDH2
INSIG2
INSR
IP6K2
IRS1
ITGA1
ITGA2
ITGA9
ITGB1
ITGB3
ITGBL1
ITIH3
ITPA
ITPKC
JAK2
KANSL1
KCNE1
KCNH2
KCNH7
KCNIP1
KCNIP4
KCNJ1
KCNJ11
KCNJ6
KCNMA1
KCNMB1
KCNQ1
KCNQ5
KCNT1
KCNT2
KDM4A
KDR
KIAA0391
KIF6
KIR2DL2
KIRREL2
KIT
KL
KLC1
KLC3
KLRC1
KLRD1
KLRK1
KRAS
KYNU
LAMB3
LARP1B
LCE3B
LCE3C
LDLR
LECT2
LEP
LEPR
LGALS3
LGR5
LIG3
LINC00251
LINC00478
LIPC
LPA
LPHN3
LPIN1
LPL
LRP1
LRP1B
LRP2
LRP5
LRRC15
LST1
LTA
LTA4H
LTB
LTC4S
LUC7L2
LYN
LYRM5
MAD1L1
MAFB
MAFK
MALAT1
MAML3
MAN1B1
MAP3K1
MAP3K5
MAP4K4
MAPK1
MAPK14
MAPT
March1
MC1R
MC4R
MCPH1
MDGA2
MDM2
MDM4
MECP2
MED12L
MEG3
MET
METTL21A
MEX3C
MGAT4A
MGMT
MIA3
MICA
MICB
MIR1206
MIR1307
MIR133B
MIR146A
MIR2053
MIR27A
MIR300
MIR423
MIR4278
MIR449B
MIR492
MIR577
MIR595
MIR604
MIR611
MIR618
MIR7-2
MISP
MLLT3
MLN
MME
MMP1
MMP10
MMP2
MMP3
MMP9
MOB3B
MOCOS
MOV10
MPO
MPZ
MS4A2
MSH2
MSH3
MSH6
MT-RNR1
MTCL1
MTHFD1
MTHFR
MTMR12
MTOR
MTR
MTRF1L
MTRR
MTTP
MUC5B
MUTYH
MVK
MYC
MYLIP
MYOCD
N6AMT1
NALCN
NANOGP6
NAT1
NAT2
NAV2
NBAS
NBEA
NCF4
NCOA1
NCOA3
NEDD4
NEDD4L
NEFM
NELFCD
NELL1
NEUROD1
NFATC1
NFATC2
NFE2L2
NFKB1
NFKBIA
NGF
NGFR
NLGN1
NLRP3
NLRP8
NOD2
NOS1AP
NOS2
NOS3
NPAS3
NPC1L1
NPHS1
NPPA
NPPA-AS1
NQO1
NQO2
NR1D1
NR1H3
NR1I2
NR1I3
NR3C1
NR3C2
NRAS
NRG1
NRG3
NRP1
NRP2
NRXN1
NT5C1A
NT5C2
NT5C3A
NT5E
NTRK1
NTRK2
NUBPL
NUDT15
NUMA1
OAS1
OASL
OCRL
OPN1SW
OPRD1
OPRK1
OPRM1
OR10AE3P
OR4D6
OR52E2
OR52J3
ORM1
ORM2
ORMDL3
OSMR
OTOS
OXT
P2RY1
P2RY12
PACSIN2
PADI4
PAPD7
PAPLN
PAPPA2
PARD3B
PARP11
PAX4
PCK1
PCSK9
PDCD1LG2
PDE4B
PDE4C
PDE4D
PDGFRA
PDGFRB
PDLIM5
PDZRN3
PEAR1
PEMT
PER2
PER3
PGLYRP4
PGR
PHACTR1
PHB2
PHTF1
PI4KA
PICALM
PICK1
PIGB
PIK3CA
PIK3R1
PITPNM2
PKLR
PLA2G4A
PLAGL1
PLCB1
PLCD3
PLCG1
PLEKHH2
PLEKHN1
PLG
PLXNB3
PMCH
POLA2
POLG
POLR3G
POMT2
PON1
PON2
POR
POU2F1
POU2F2
POU5F1
PPARA
PPARD
PPARG
PPARGC1A
PPFIA1
PPM1A
PPP1R13L
PPP1R1C
PPP2R5E
PRB2
PRCP
PRDM1
PRDM16
PRDX4
PRIMPOL
PRKAA1
PRKAA2
PRKCA
PRKCB
PRKCE
PRKCQ
PRKG1
PROC
PROCR
PROM1
PROS1
PROX1
PRRC2A
PRSS53
PSMA4
PSMB3P
PSMB4
PSMB8
PSMD14
PSORS1C1
PSORS1C3
PSRC1
PTCHD1
PTEN
PTGER2
PTGER3
PTGER4
PTGES
PTGFR
PTGIR
PTGS1
PTGS2
PTH
PTH1R
PTPN22
PTPRC
PTPRD
PTPRM
PTPRN2
PYGL
RAB27A
RABEPK
RAC2
RAD18
RAD52
RAF1
RALBP1
RAPGEF5
RARG
RARS
RBFOX1
RBMS3
REEP5
REL
REN
REPS1
RET
REV1
REV3L
RFK
RGS17
RGS2
RGS4
RGS5
RHBDF2
RHOA
RICTOR
RND1
RNFT2
RORA
RPL13
RRAS2
RRM1
RRM2
RRM2B
RSBN1
RSRP1
RUNX1
RXRA
RYR1
RYR2
RYR3
SACM1L
SCAP
SCARB1
SCGB3A1
SCN10A
SCN1A
SCN2A
SCN4A
SCN5A
SCN8A
SCN9A
SCNN1B
SCNN1G
SELE
SELP
SEMA3C
SERPINA3
SERPINA6
SERPINE1
SERPINF1
SERPING1
SETD4
SFRP5
SH2B3
SH2D5
SH3BP2
SHMT1
SIK3
SIN3A
SKIV2L
SKOR2
SLC10A2
SLC12A3
SLC12A8
SLC14A2
SLC15A1
SLC15A2
SLC16A5
SLC16A7
SLC17A3
SLC18A2
SLC19A1
SLC1A1
SLC1A2
SLC1A3
SLC1A4
SLC22A1
SLC22A11
SLC22A12
SLC22A16
SLC22A17
SLC22A2
SLC22A3
SLC22A4
SLC22A5
SLC22A6
SLC22A7
SLC22A8
SLC24A4
SLC25A13
SLC25A14
SLC25A27
SLC25A31
SLC26A9
SLC28A1
SLC28A2
SLC28A3
SLC29A1
SLC2A1
SLC2A2
SLC2A9
SLC30A8
SLC30A9
SLC31A1
SLC37A1
SLC39A14
SLC47A1
SLC47A2
SLC5A2
SLC5A7
SLC6A12
SLC6A2
SLC6A3
SLC6A4
SLC6A5
SLC6A9
SLC7A5
SLC7A8
SLCO1A2
SLCO1B1
SLCO1B3
SLCO1C1
SLCO2B1
SLCO3A1
SLCO4C1
SLCO6A1
SLIT1
SMARCAD1
SMYD3
SNAP25
SNORA59B
SNORD68
SOCS3
SOD2
SOD3
SORT1
SOX10
SP1
SPARC
SPATS2L
SPECC1L
SPG7
SPIDR
SPINK5
SPP1
SPTA1
SQSTM1
SREBF1
SREBF2
SRP19
SRR
ST13
STAT3
STAT4
STAT6
STIM1
STIP1
STK39
STMN1
STMN2
STX1B
STX4
SUGCT
SULT1A1
SULT1A2
SULT1C4
SULT1E1
SULT2B1
SV2C
SYN3
SYNE3
SZRD1
T
TAAR6
TAC1
TAGAP
TANC1
TANC2
TAP1
TAP2
TAPBP
TAS2R16
TBC1D1
TBC1D32
TBX21
TBXA2R
TBXAS1
TCF19
TCF7L2
TCL1A
TDP1
TDRD6
TERT
TET2
TF
TGFB1
TGFBR2
TGFBR3
TH
THBD
THRA
THRB
TIGD1
TK1
TLR2
TLR3
TLR4
TLR5
TLR7
TLR9
TMCC1
TMCO6
TMEFF2
TMEM205
TMEM258
TMEM57
TMPRSS11E
TNF
TNFAIP3
TNFRSF10A
TNFRSF11A
TNFRSF11B
TNFRSF1A
TNFRSF1B
TNFSF10
TNFSF11
TNFSF13B
TNRC6A
TNRC6B
TOLLIP
TOMM40
TOMM40L
TOP1
TOP2B
TP53
TPH1
TPH2
TPMT
TRAF1
TRAF3IP2
TRIB3
TRIM5
TRPM6
TSC1
TSPAN5
TTC6
TUBB1
TUBB2A
TXNRD2
TYMP
TYMS
UBASH3B
UBE2I
UCP2
UCP3
UGGT2
UGT1A
UGT1A1
UGT1A10
UGT1A3
UGT1A4
UGT1A5
UGT1A6
UGT1A7
UGT1A8
UGT1A9
UGT2B10
UGT2B15
UGT2B17
UGT2B4
UGT2B7
ULK3
UMPS
UPB1
USH2A
USP24
USP5
UST
VAC14
VASP
VDR
VEGFA
VKORC1
WBP2NL
WBSCR17
WDR7
WIF1
WNK1
WNT5B
WT1
WWOX
XBP1
XDH
XPA
XPC
XPO1
XPO5
XRCC1
XRCC3
XRCC4
XRCC5
YAP1
YBX1
YEATS4
ZBTB22
ZBTB4
ZCCHC6
ZFP91-CNTF
ZMAT4
ZNF100
ZNF215
ZNF423
ZNF432
ZNF652
ZNF697
ZNF804A
ZNF816
ZNRD1-AS1
ZSCAN25

TABLE 4
Clinical Testing Genes
Gene (HGNC Symbol)

LMNA
PTEN
TP53
BRCA2
MLH1
MSH2
BRCA1
MSH6
FGFR3
MECP2
CFTR
RET
PTPN11
SCN5A
MYH7
CAV3
PMS2
KRAS
APC
ATM
ARX
DMD
DES
STK11
POLG
NF1
BRAF
TSC1
CDKL5
TSC2
TTN
COL2A1
FMR1
FKTN
KCNQ1
VHL
SLC2A1
FBN1
EPCAM
HRAS
PALB2
RAF1
TNNT2
CEP290
SMAD4
MUTYH
SCN1A
SCN1B
KCNJ2
RYR2
GLA
CDH1
NRAS
FKRP
KCNH2
LDB3
CACNA1A
MYBPC3
FGFR2
UBE3A
CACNA1C
GJB2
TAZ
SDHB
TNNI3
ACTC1
GAA
TCAP
CHEK2
LAMP2
COL1A1
TTR
DSP
HBB
SDHD
SOS1
NBN
COL1A2
TGFBR2
POMT1
TPM1
FLNA
KCNE1
PCDH19
MAP2K1
CHD7
FOXG1
SDHC
TGFBR1
RYR1
MTHFR
SGCD
CDKN2A
PMP22
POMT2
FH
WT1
EMD
SCN4A
FGFR1
PLP1
PAX6
POMGNT1
TMEM43
MEN1
PKP2
SLC9A6
RHO
F5
GCK
BRIP1
TRIM32
DSG2
RAD51C
TRPV4
SCN2A
CPT2
KCNE2
GJB6
COL3A1
MAP2K2
NPHP1
DNM2
BMPR1A
PRKAG2
ACADM
OFD1
MYOT
CASQ2
HEXA
DSC2
MEF2C
HFE
CLN3
PTCH1
CRYAB
JUP
PLN
MED12
ZEB2
FHL1
ABCC8
F2
ACADVL
BAG3
ATP7A
CASR
SCN9A
BSCL2
PDHA1
SHOC2
ETFDH
KCNQ2
HADHA
TNNC1
PRRT2
TPP1
ANO5
COL5A1
ETFB
MPZ
ETFA
ACTA1
PPT1
CASK
STXBP1
ABCD1
KCNJ11
ATRX
GNAS
ABCA4
DYSF
ABCC9
TCF4
BLM
SLC22A5
SDHA
MYH6
HCN4
ATP7B
PLA2G6
FANCC
MYL2
CBS
ANK2
KCNE3
MYL3
CLN5
DCX
PANK2
ALDH7A1
NKX2-5
GBA
TIMM8A
PNKP
ACTA2
WFS1
MFN2
FOLR1
JAG1
SMN1
SMARCB1
L1CAM
GPC3
KIT
NSD1
OPA1
DHCR7
NF2
SGCA
MITF
CLRN1
TPM2
SPRED1
MKS1
NIPBL
AGL
OTC
RB1
CSRP3
GLB1
TMEM67
CLN6
HNF1B
SMC1A
SCN4B
CACNB2
ACVRL1
DLD
CBL
FXN
ARSA
PSEN1
COL6A3
LAMA2
SMAD3
ENG
PRPS1
ACTN2
TWNK
CAPN3
GDAP1
COL5A2
EYA1
PCDH15
GCH1
SURF1
SGCB
SCN3B
TMEM216
PITX2
COL6A1
PEX1
MYH11
VCL
NOTCH3
LARGE1
SLC26A4
CLN8
BTD
GAMT
USH2A
MYH9
AR
NPC1
TERT
GABRG2
GCDH
HNF1A
FLNC
IDS
COL6A2
BBS1
RPGR
FLCN
GNE
RPGRIP1L
MEFV
CALM1
CDKN1C
MFSD8
PRPH2
SMPD1
OPHN1
CNTNAP2
BCKDHB
PLOD1
PLEC
CREBBP
SDHAF2
ARHGEF9
AKAP9
RAD51D
NEB
OPA3
MBD5
NPC2
MYO7A
CTSD
VPS13B
GALC
KCNJ5
PAFAH1B1
PYGM
GRN
ASPA
CDK4
PEX7
MET
FBN2
CC2D2A
GARS
NRXN1
PIK3CA
COL11A2
HTT
SLC26A2
SETX
NEXN
TGFB3
SELENON
KCNJ10
CPT1A
HPRT1
ELN
UGT1A1
WAS
OCRL
KCND3
MUT
VCP
HADHB
GPD1L
KCNQ3
SUCLA2
SCO2
FTL
EGR2
PMM2
ALPL
SNTA1
BBS2
G6PC
HADH
PKD2
PKHD1
COQ2
MMACHC
GJB1
BEST1
SGCG
BCKDHA
LDLR
NPHP3
SLC25A20
ACADS
DYNC1H1
KCTD7
MAPT
FIG4
TREX1
MMAB
PQBP1
GRIN2A
COL4A5
MMAA
MKKS
RPE65
GBE1
NDP
HSD17B10
GATA1
APOB
TTC8
SPG7
PDX1
GABRA1
APTX
IKBKAP
NEFL
PEX6
COL11A1
TBC1D24
TGFB2
CRX
APOE
GUCY2D
PHOX2B
ISPD
ATP1A2
ATP13A2
ATL1
SYNE1
ATXN2
SLC6A8
ALMS1
HNF4A
AHI1
ACAD9
PRKAR1A
SNRPN
COL4A1
NOTCH1
SLC25A22
GLDC
ADGRV1
GALT
PEX26
TRDN
PHF6
PNPO
KCNT1
MTM1
COX15
SLC4A1
RRM2B
PRSS1
TPM3
BBS10
BAP1
BCS1L
CDH23
MRE11
PCCA
TBX5
MPL
PAH
SPTAN1
SCN8A
AMT
ASS1
PSEN2
CACNA1S
USH1C
FANCA
CYP21A2
FGD1
PEX12
SLC2A10
WDR62
FAH
GLI3
RUNX1
ANKRD1
GNPTAB
SLC25A4
SERPINA1
RELN
BARD1
RAPSN
DKC1
CSTB
SGCE
F8
KCNJ8
MYPN
MVK
PEX10
REEP1
CRB1
CHRNA1
RBM20
PCCB
BCOR
NLRP3
HBA1
EPM2A
SKI
GATA2
MYLK
FANCB
TYR
ABCB4
C12orf65
PEX2
LRP5
TTC21B
SLC25A13
HSPB1
HSPB8
MPV17
SPAST
SLC37A4
IQCB1
IDUA
EYA4
KCNA1
PGK1
CYP1B1
WHRN
SMARCA4
TERC
ADSL
DMPK
ATXN1
ATP6AP2
SYNGAP1
RDH12
TARDBP
KMT2D
PRKN
NPHP4
TK2
NHLRC1
GJA1
SUCLG1
GATA4
NDUFA1
COL4A3
ATXN3
VWF
TH
DBT
KIF1A
MMADHC
MID1
PKD1
AP3B1
CHRNA4
DNAJB6
APP
SHH
FA2H
CHRNB2
EDN3
SLC16A2
ELANE
FUS
INS
RPS6KA3
INVS
MYOZ2
TNNT1
ALK
TMEM70
CACNB4
JAK2
CNGB3
SPINK1
AGXT
PAX3
MCOLN1
PEX5
ASPM
DGUOK
IGHMBP2
CFH
SOD1
TUBA1A
DOLK
PROM1
SYN1
HMGCL
KDM5C
RAB39B
DNAJC5
AUH
SHOX
ATXN7
CENPJ
SRPX2
SOX10
CYP2D6
DCTN1
TBX1
ALDOB
ARL6
BBS12
COQ8A
TWIST1
RECQL4
OTX2
PC
DPAGT1
TP63
GP1BA
ARG1
POLD1
SACS
AKT1
PEX3
SMC3
OCA2
CYP2C19
RMRP
IL2RG
DNAH5
SPG11
NDRG1
COL4A4
FOXC1
BMPR2
MCCC2
MAX
F9
ERCC6
C9orf72
TYMP
RAI1
AIPL1
MCCC1
SLC25A19
COL9A1
BTK
P3H1
PDSS2
PCNT
NOTCH2
ATP8B1
ATP1A3
ETHE1
HEXB
SLC25A15
CP
COL9A2
CHRNA2
CHRNE
CUL4B
DOK7
CHRND
GUSB
SLC19A3
IVD
SH3TC2
EFHC1
IMPDH1
CRTAP
CYP27A1
HSPD1
SOX2
SDCCAG8
CYP2C9
ALS2
RPS19
GOSR2
RARS2
GFAP
PEX14
CYP11B1
GMPPB
BBS4
SGSH
GJC2
GLUD1
GATM
TMEM127
RPGRIP1
PDGFRA
LGI1
MT-ATP6
ADAMTS13
BBS5
WDR45
MTMR2
GATA6
BBS7
LITAF
POLG2
ABCB11
PRX
ALG2
ABCC6
RNASEH2B
FANCG
ADA
SIL1
RP2
RASA1
NTRK1
TNFRSF1A
SCNN1B
CHAT
USH1G
FLNB
DNAI1
CFL2
OPTN
NDUFS4
ARL13B
BBS9
TOR1A
LRPPRC
ATPAF2
SAMHD1
TSEN54
NPHS2
TSFM
HBA2
GALNS
FKBP14
CHST14
FOXRED1
TRPM4
NHS
RNASEH2A
RNASEH2C
ADGRG1
MT-RNR1
AGK
CEP152
ASL
SNCA
GRIN2B
DTNA
SIX1
CPS1
KIF7
AIFM1
PDHX
NAGLU
MT-TL1
NSDHL
HDAC8
HGSNAT
LRRK2
SBF2
RAB7A
SCNN1G
LRAT
DARS2
KIF5A
RIT1
PCSK9
GFM1
PINK1
NPHS1
ARSB
NDUFS7
POLE
PFKM
SCN2B
IDH2
FBLN5
INPP5E
PDSS1
GABRD
ATP6V0A2
PRICKLE1
ACAT1
SOX9
CACNA2D1
G6PD
SPG20
SCARB2
NLGN3
ANOS1
NLGN4X
GABRB3
HAX1
AFG3L2
GJB3
TINF2
KRIT1
GPR143
CDC73
EDNRB
MLYCD
AARS2
JAK3
SDHAF1
JPH2
NDUFV1
PEX13
PLCB1
ABHD12
PEX16
IRF6
SUMF1
BSND
DAG1
HLCS
ATR
EGFR
AFF2
EZH2
PEX19
ABCA3
PAK3
NDUFS1
PHYH
PRKCG
TMPO
TULP1
COMP
MPI
MYLK2
HESX1
YARS
BIN1
DPM3
LYST
AARS
SIX3
ACTG1
C19orf12
PDHB
COQ9
MLC1
NODAL
DPYD
CHM
DPM1
LIPA
SFTPC
DLAT
VRK1
TUBB2B
ATP6V1B1
HSD17B4
CERKL
EP300
SLC12A3
GATA3
FANCE
FGD4
CFI
SCN10A
COLQ
COX6B1
FKBP10
EXT1
ADAMTS2
SBDS
CD46
TGIF1
SALL1
ERCC4
KIF1B
SLC17A5
WNK1
KCNA5
ARFGEF2
FANCF
ELOVL4
SALL4
CYP7B1
KARS
GRIA3
ALDH5A1
SPR
CLCN1
HCCS
GNS
EIF2AK3
PUS1
PDE6B
PLOD2
PAX2
DHDDS
WDR19
ALG6
PPARG
VAPB
CHD2
RP1
PSAP
WRN
LMBRD1
INSR
CEBPA
LPIN1
SMS
MT-TK
PARK7
SUFU
UMOD
PRNP
AGA
RAD50
FUCA1
SLC39A13
NDUFA2
ISCU
MT-TS1
SEMA4A
FOXP3
TACO1
LIG4
AIRE
SRY
KBTBD13
EIF2B5
MT-ND1
IKBKG
DICER1
TRMU
MUSK
SLC25A3
OTOF
POMK
TBP
RAG2
UPF3B
EDA
RLBP1
RAB3GAP1
LAMB2
CEP41
RAD21
KDM6A
MCPH1
CABP4
SPATA7
MTRR
LAMA4
EFEMP2
NDUFS8
GALK1
SAG
LCA5
NR2E3
EXT2
GCSH
PPIB
PORCN
EHMT1
CTNNB1
CTNS
TFR2
C3
HCN1
EIF2B1
SLX4
POU3F4
WDPCP
INF2
LIAS
CHRNB1
ACTB
AP1S2
PHEX
SPTB
NEUROD1
RS1
NPPA
SOX3
FGF23
MAN2B1
DNAH11
ERCC2
DGKE
CCM2
NDUFAF2
EVC
RAG1
HPS1
NDUFS3
NDUFS2
ZIC2
FGF8
LPL
FASTKD2
TCTN2
CACNA1D
HPS4
CACNA1F
CLCN5
GJA5
SYP
GP1BB
FANCL
ACSL4
IDH1
CLCNKB
CISD2
ROR2
NEU1
GATAD1
MYH3
NDE1
PRPF31
ABCG5
NKX2-1
PGM1
TMEM237
FBP1
CDK5RAP2
NDUFAF5
ZFYVE26
DPM2
PHKA1
MT-ND6
STIL
TUBB3
BICD2
IQSEC2
SPTA1
ITGA7
QDPR
TJP2
PTS
EIF2B3
NOD2
GLRA1
CSF1R
PRF1
ATN1
PAX4
GPSM2
CHMP2B
CFB
EYS
FANCI
ST3GAL3
AGPAT2
PDP1
IL7R
HK1
PNPLA2
RAB27A
DCLRE1C
MC4R
GYS2
B9D1
SCNN1A
ANG
ENPP1
PRPF8
SFTPB
FANCM
AXIN2
LMX1B
NHEJ1
SYNE2
TTC19
PROP1
MAGT1
COL7A1
FANCD2
FSCN2
NDUFAF1
MT-ND4
KCNJ1
COL12A1
CNGA3
STAT3
TYRP1
NDUFS6
GUCA1B
SLC2A2
SIX5
ADAR
SLC33A1
CCDC39
AMACR
GAN
HFE2
B3GLCT
EFNB1
UQCRB
SLC12A6
FGA
HPS3
XRCC2
MTR
C8orf37
ACTN4
EVC2
THAP1
TRPS1
IDH3B
RUNX2
LAMB3
SH2D1A
GDI1
TMC1
DNMT1
PDCD10
MRPS22
LAMA3
TOPORS
CHKB
MTPAP
CYP17A1
POMGNT2
SLC12A1
ZIC3
GLI2
RD3
ALAS2
RPL35A
CNGB1
LDLRAP1
DEPDC5
THBD
DYRK1A
SLC19A2
DNAI2
PGAM2
PNKD
ASAH1
WDR35
VKORC1
DOCK8
PHGDH
SLC45A2
GP9
CCDC78
SPTLC1
IL1RAPL1
SLC35C1
UBE2A
NR0B1
CAVIN1
ACOX1
AGRN
CA4
COL9A3
CNGA1
LAMC2
DTNBP1
EIF2B2
TTPA
FLVCR1
MYH14
ERBB2
ITGB3
VLDLR
WASHC5
NDUFA11
C2orf71
PTCHD1
NRL
ALDH4A1
RSPH9
ATP5E
GK
CTDP1
ABL1
TCTN1
ANK1
CTSA
SLC40A1
AKT3
B4GAT1
ZMPSTE24
MERTK
EIF2B4
ERCC8
NUBPL
PPOX
PDLIM3
PNPLA6
TNXB
PRKG1
FOXH1
COG7
RPL11
GPHN
ABCG8
PDE6C
B4GALT7
G6PC3
GNA11
CLCN2
NME8
KCNJ13
HEPACAM
SLCO1B1
UQCRQ
NDUFAF4
TMEM138
MT-ND5
NDUFAF3
HMBS
NHP2
IFITM5
MBTPS2
SMN2
PDE6A
VSX2
MYO6
CPOX
ALG13
CCDC40
ALDH3A2
NIPA1
TSHR
ZNF423
SQSTM1
MOCS2
L2HGDH
SCO1
TUBB4A
TCOF1
MOCS1
MTO1
CIB2
HINT1
KIAA2022
ERCC3
PITX3
PRPF3
DNM1L
TCTN3
FHL2
CA2
GRHPR
PLEKHG5
CDON
KLHL40
TSEN2
SLC1A3
RGR
NEBL
C5orf42
HPS6
GFI1
MYCN
LZTR1
BRWD3
TSEN34
F11
SNRNP200
GNAT2
ALG1
TMEM126A
SP7
KLHL7
TUFM
DLG3
DNAAF2
DNAAF1
VPS13A
NOP10
TMEM5
MCEE
STXBP2
MED25
SHANK3
SLC3A1
TECTA
COX10
CHRNG
RDH5
CDHR1
PHF8
RPL5
MAOA
GFPT1
RAB3GAP2
CALM2
NAGS
POLR1C
HSD3B2
AMPD1
BUB1B
NEK8
TUBA8
B3GALNT2
FLT3
MATR3
KRT5
GDF6
GREM1
AVPR2
DNAL1
ZDHHC9
CTC1
ALDOA
NR5A1
CYBB
FTSJ1
BLOC1S3
EBP
DCAF17
SPG21
ACAD8
ABCB7
F12
GLRB
GLIS2
EXOSC3
HUWE1
BMP4
TMIE
GNPTG
RPS26
ITGA2B
LRSAM1
SLC6A3
ALDH18A1
SERPINC1
KLF11
F7
RPS10
WNT10A
NFIX
MGAT2
ACSF3
RBBP8
CFHR5
COQ6
UBQLN2
CDKN1B
SUOX
FAM126A
COG8
NDUFA10
SMARCE1
ALG8
GSS
EPB42
RPL10
DNAJC19
NAA10
KCNMA1
RPS24
STX11
ALG3
XK
MFRP
TMPRSS3
TSPAN7
SERPINH1
IMPG2
ALG12
SERPINE1
SLC16A1
TCIRG1
STIM1
ETV6
CLCN7
GDF2
SLC35A1
FAM161A
ARID1B
TMEM231
SLC35A2
NGF
COX4I2
POU1F1
GLIS3
TAF1
PNP
POMC
KIF1BP
BLK
YARS2
TCN2
UNC13D
HAMP
HOGA1
ACADSB
B4GALT1
MANBA
KAT6B
RSPH4A
ACE
EDAR
WWOX
FARS2
GNAQ
GNPAT
ANKH
ENO3
FRAS1
RANGRF
GALE
TREM2
CD3D
LEP
TFG
IER3IP1
DYNC2H1
NPM1
KMT2A
CD40LG
PYGL
MT-CYB
DFNB59
MRPS16
RTN2
KCNE5
MATN3
TAT
NDUFV2
CDAN1
STS
CAV1
B3GALT6
CTSK
CALR3
KCNV2
AP4M1
SERPING1
GYS1
HPS5
ST3GAL5
SLC6A5
ARID1A
PRKRA
COG1
COL4A2
EFEMP1
PIK3R2
MTFMT
SEPT9
FOXP1
NDUFAF6
ROM1
KRT14
SLC25A12
SEC23B
TNNI2
CD3E
HPD
PHKB
AIP
FZD4
XPNPEP3
CEP164
ITGB4
SLMAP
PABPN1
TBCE
GHR
NOG
CACNA2D4
ALG9
FOXL2
TYROBP
THRB
AP4E1
BDNF
AKT2
DSPP
MPDU1
EDARADD
TPMT
SPTBN2
BLOC1S6
FGF14
CTSF
PRCD
SRD5A3
PRPF6
TRAPPC11
PHKA2
COCH
AGPS
EARS2
FOXE3
IGBP1
RBP3
PKLR
PIGA
MAT1A
SPTLC2
CEP63
FBXO7
SETBP1
OTOA
RTEL1
PTF1A
LEPR
SMARCAL1
SCP2
PCBD1
DMP1
MOGS
CNTN1
TNPO3
POLR3A
SLC46A1
FOXI1
MYO15A
KCNQ4
MYOC
PYCR1
APOA5
GRHL2
POR
AICDA
KISS1R
PRDM16
ARSE
LHFPL5
PDE6G
HARS
SNAI2
VCAN
SMPX
CSF3R
COL17A1
LOXHD1
MTTP
SERPINF1
PROKR2
GNRHR
D2HGDH
B9D2
ZAP70
AP5Z1
CTNNA3
CSF2RA
SLC34A3
ZNF513
TNFRSF11A
CTRC
RP9
HSPG2
KANSL1
RPS7
TRIOBP
CEL
SHROOM4
SLC7A7
RFT1
ADAMTSL4
ABCA12
ABAT
LPIN2
ERCC5
HGF
PROC
LHX4
ROGDI
ABCA1
DIABLO
ESCO2
PRDM5
PHKG2
FREM1
PRODH
DIS3L2
RDX
WRAP53
MC1R
ACVR1
ZNF711
IFT80
ACVR2B
EFTUD2
LTBP2
MEGF10
RAB18
CLDN14
FLT4
CCT5
SRCAP
ESRRB
PDZD7
NEK1
NR3C2
TBX20
DNAJB2
FAS
ATXN10
CFHR1
GDF5
PSTPIP1
ARHGEF6
TDP1
GUCA1A
OXCT1
PPP2R2B
AQP2
TRPC6
MARVELD2
FECH
OAT
PEX11B
PRICKLE2
APOC2
PDGFRB
CACNA1H
LHCGR
SARS2
LRTOMT
COL10A1
XIAP
UNG
MGME1
SLC26A5
CYBA
PITPNM3
PTH1R
TIMP3
DRD2
PDE6H
ALX4
TXNRD2
OBSL1
ORC1
GH1
CSPP1
LEFTY2
CCDC50
ABCD4
DIAPH1
CDH3
CHCHD10
PAX8
GDNF
MT-CO1
HARS2
HTRA1
BMP1
MSRB3
ZDHHC15
CAVIN4
AP4S1
CFHR3
ACADL
NDUFA9
MSX1
MYO3A
CYP11B2
CTF1
MAK
AP4B1
IFT122
ABHD5
MARS
A2ML1
CHST3
CYLD
GDF1
XPA
MT-TH
TPRN
MT-TQ
POU4F3
XPC
GRIN1
GIPC3
CYP27B1
POLR1D
LHX3
TGFB1
TOR1AIP1
CNBP
GM2A
DDHD2
TRPM1
BCKDK
DNAAF3
HSD11B2
ADAM9
CLCNKA
NDUFB3
LAS1L
MAGI2
ANKRD11
NMNAT1
ZFYVE27
DNMT3A
PROK2
SMARCA2
GFER
POLR3B
NDUFA12
PLCE1
STRA6
EMX2
HMGCS2
ASCL1
COMT
PROS1
KCNC3
ILK
FGB
C10orf11
ILDR1
ANKRD26
GRXCR1
SZT2
HNRNPDL
KIF11
FGG
DDC
TTBK2
FREM2
ZNF469
TUSC3
TFAP2A
DLL3
CLIC2
GDF3
MT-TS2
CYP3A5
AHCY
LDHA
SLC52A3
PRKCSH
ACY1
ACO2
KCNK3
AMER1
WNT1
MARS2
NYX
VPS35
UROS
COG6
REN
AVP
MTOR
TBX3
RBM10
PFN1
TPO
MYBPC1
SERPINB6
PTPRC
H19
ABCB6
WNT7A
MYO5A
CCDC88C
ATP6V0A4
OSTM1
SRD5A2
CDT1
DFNA5
ESPN
MYF6
USB1
DDOST
CRYM
APOA1
ATXN8OS
AGTR2
SLC17A8
MSX2
DST
LTBP4
KLHL3
AAAS
RFX6
LBR
CYP3A4
F13A1
RAX2
RAC2
PREPL
ERLIN2
ANK3
NFU1
LRP4
TNFRSF13B
TNFSF11
SNAP29
LAMC3
RBM8A
ORC6
GRM6
COG5
ORC4
PDYN
CRELD1
SLC5A7
ITGA3
SPINK5
WNT4
ENAM
C1QTNF5
PDK3
HTRA2
GNB4
WNK4
COG4
MT-TI
HSPB3
MT-TL2
HCFC1
POT1
ICOS
SIGMAR1
ATP2A1
GNAT1
SOS2
CTSC
FOXP2
TMEM165
CXCR4
SH3BP2
TACR3
CFC1
ABCC2
DNAJC6
DHODH
CPA6
AK2
HOXD13
VPS45
PLOD3
KRT1
MT-ATP8
DNAAF5
TGM1
TSPAN12
IFT172
CD2AP
MRPL3
LIFR
RIMS1
CNNM4
CDC6
F10
FOXC2
STAT5B
PIK3R1
ORAI1
ZNF81
ZFP57
CYP24A1
GLE1
COL18A1
TIA1
RPL26
GNAO1
LCAT
VDR
ANO10
TNNT3
LZTFL1
COL4A6
SHANK2

### REFERENCES

Aoki et al., “The RAS/MAPK Syndromes: Novel Roles of the RAS Pathway in Human Genetic Disorders,” Human Mutation, 2008.
KARCZEWSKI et al., “Analysis of protein-coding genetic variation in 60,706 humans,” Nature, 2016.
LANDRUM et al., “ClinVar: public archive of interpretations of clinically relevant variants,” Nucleic Acids Res., 2015.
MAXWELL et al., “Evaluation of ACMG-Guideline-Based Variant Classification of Cancer Susceptibility and Non-Cancer-Associated Genes in Families Affected by Breast Cancer,” Am. J. Hum. Genet., 2016.
MYERS et al., “The lipid phosphatase activity of PTEN is critical for its tumor supressor function,” Proc. Natl. Acad. Sci. U.S.A., 1998.
MYERS et al., “P-TEN, the tumor suppressor from human chromosome 10q23, is a dual-specificity phosphatase,” Proc. Natl. Acad. Sci. U.S.A, 1997,
H E et al., “Cowden syndrome-related mutations in PTEN associate with enhanced proteasome activity,” Cancer Res., 2013.
HEIKKINEN et al., “Variants on the promoter region of PTEN affect breast cancer progression and patient survival,” Breast Cancer Res., 2011.
JOHNSTON et al., “Conformational stability and catalytic activity of PTEN variants linked to cancers and autism spectrum disorders,” Biochemistry, 2015.
MARKKANEN et al., “DNA Damage and Repair in Schizophrenia and Autism: Implications for Cancer Comorbidity and Beyond,” Int. Sci., 2016.
SCHARNER et al., “Genotype-phenotype correlations in larninopathies: how does fate translate?,” Biochem. Soc. Trans., 2010.
ARAYA et al., “Deep mutational scanning: assessing protein function on a massive scale,” Trends Biotechnol., 2011.
SHENDURE et al., “Massively Parallel Genetics,” Genetics, 2016.
KELSIC et al., “RNA Structural Determinants of Optimal Codons Revealed by MAGE-Seq,” Cell Cyst, 2016.
PATWARDHAN et al., “High-resolution analysis of DNA regulatory elements by synthetic saturation mutagenesis,” Nat. Biotechnol., 2009.
BIJENROSTRO et al., “Quantitative analysis of RNA-protein interactions on a massively parallel array reveals biophysical and evolutionary landscapes,” Nat. Biotechnol., 2014.
GUENTHER et al., “Hidden specificity in an apparently nonspecific RNA-binding protein,” Nature, 2013.
ARAYA et al., “A fundamental protein property, thermodynamic stability, revealed solely from large-scale measurements of protein function,” Proc. Natl. Acord. Sci. U.S.A., 2012.
FOWLER et al., “High-resolution mapping of protein sequence-function relationships,” Nat. Methods, 2010.
MAJITHIA et al., “Prospective functional classification of all possible missense variants in PPARG,” Nat, Genet, 2016.
STARITA et al., “Massively Parallel Functional Analysis of BRCA1 RING Domain Variants,” Genetics, 2015.
BUENROSTRO et al., “Single-cell chromatin accessibility reveals principles of regulatory variation,” Nature, 2015.
CUSANOVICH et al., “Multiplex single-cell profiling of chromatin accessibility by combinatorial cellular indexing,” Science, 2015.
CAO et al., “Comprehensive single cell transcriptional profiling of a multicellular organism by combinatorial indexing,” bioRxiv, 2017.
ZHENG et al., “Massively parallel digital transcriptional profiling of single cells,” Nat. Connnun., 2017.
DATLINGER et al., “Pooled CRISPR screening with single-cell transcriptome readout,” Nat. Methods, 2017.
JAITIN et al., “Dissecting Immune Circuits by Linking CRISPR-Pooled Screens with Single-Cell RNA-Seq,” Cell, 2016.
ADAMSON et al., “A Multiplexed Single-Cell CRISPR Screening Platform Enables Systematic Dissection of the Unfolded Protein Response,” Cell, 2016.
DIXIT et al., “Perturb-Seq: Dissecting Molecular Circuits with Scalable Single-Cell RNA Profiling of Pooled Genetic Screens,” Cell, 2016.
MACOSKO et al., “Highly Parallel Genome-wide Expression Profiling of Individual Cells Using Nanoliter Droplets,” Cell, 2015.
GAWAD et al., “Single-cell genome sequencing: current state of the science,” Nat. Rev. Genet., 2016.
TANAY et al., “Scaling single-cell genomics from phenomenology to mechanism,” Nature, 2017.
SCHWARTZMAN et al., “Single-cell epigenomics: techniques and emerging applications,” Nat. Rev. Genet., 2015,
BUZDIN et al., “The OncoFinder algorithm for minimizing the errors introduced by the high-throughput methods of transcriptome analysis,” Front Mol Biosci, 2014.
MACOSKO et al., “Highly Parallel Genome-wide Expression Profiling of Individual Cells Using Nanoliter Droplets,” Cell, 2015.
WHITFIELD et al., “Identification of genes periodically expressed in the human cell cycle and their expression in tumors,” Mol. Biol. Cell, 2002.
PAN et al., “Using input dependent weights for model combination and model selection with multiple sources of data,” Stat. Sin., 2006.
EFRON et al., “Improvements on Cross-Validation: The 632+ Bootstrap Method,” J. Am. Stat. Assoc., 1997.
EFRON, “How Biased is the Apparent Error Rate of a Prediction Rule?,” J. Am. Stat. Assoc., 1986.
EFRON, “Estimating the Error Rate of a Prediction Rule: Improvement on Cross-Validation,” J. Am. Stat. Assoc., 1983.
SHEN et al., “Adaptive Model Selection and Assessment for Exponential Family Distributions,” Technometrics, 2004.
SHEN et al., “Adaptive Model Selection,” J Am. Stat. Assoc., 2002.
GEORGE et al., “Calibration and Empirical Bayes Variable Selection,” Biometrika, 2000.
RIPLEY et al., “Pattern Recognition and Neural Networks,” Cambridge University Press, 2008.
HASTIE et al., “The Elements of Statistical Learning. Data Mining, Inference, and Prediction,” Springer, 2001.
BURNHAM et al., “Model Selection and Multimodel Inference: A Practical Information-Theoretic Approach,” Springer, 2003.
YUVAL, “Bootstrapping with Noise: An Effective Regularization Technique,” Connection Science, 1996.
AMENDOLA et al., “Performance of ACMG-AMP Variant-Interpretation Guidelines among Nine Laboratories in the Clinical Sequencing Exploratory Research Consortium,” Am. J. Hum, Genet., 2016.
BERGER, et al., “High-throughput Phenotyping of Lung Cancer Somatic Mutations,” Cancer Cell, 2016 30(2); pp. 214-228.
MACOSKO, et al, “Highly Parallel Genome-wide Expression Profiling of Individual Cells Using Nanoliter Droplets,” Cell, 2015 161(5); pp. 1202-1214.
STARITA et al., “Deep Mutational Scanning: A Highly Parallel Method to Measure the Effects of Mutation on Protein Function,” Cold Spring Harb Protoc, 2015(8); pp. 711-714.
SHENDURE et al., “A framework for determining the relative effect of genetic variants,” U.S. patent application Ser. No. 15/023,355, filed Mar. 18, 2016.
REGEV et al., “A droplet-based method and apparatus for composite single-cell nucleic acid analysis,” International Patent Publication No. WO 2016/040476, published Mar. 17, 2016.
KALIA S S, et al, “Recommendations for reporting of secondary findings in clinical exome and genome sequencing, 2016 update (ACMG SF v2.0): a policy statement of the American College of Medical Genetics and Genomics,” Genet Med., 2016.
FUTREAL A P, et al., “A census of human cancer genes,” Nat Rev Cancer, 2004 4(3); pp. 177-183.
LAWRENCE M S, et al., “Discovery and saturation analysis of cancer genes across 21 tumour types,” Nature, 2014 505(7484); pp. 495-501.
WHIRL-CARRILLO et al., “Pharmacogenomics knowledge for personalized medicine,” Clin Pharmacol Ther, 2012 92(4); pp. 414-417.
RUBINSTEIN et al., “The NIH genetic testing registry: a new, centralized database of genetic tests to enable access to comprehensive information and improve transparency,” Nucleic Acids Res, 2013 4; pp. D925-35.
SAMOCHA K E, et al. (2017) “Regional missense constraint improves variant deleteriousness prediction,” bioRxiv:148353.
Kitzman, J. O., Starita., L. M., Lo, R. S., Fields. S. & Shendure, J. Massively parallel single-amino-acid mutagenesis. Nat. Methods 12, 203-206 (2015).
Findlay, G. M., Boyle, E. a., Hause, R. J., Klein, and Shendure, J. (2014). Saturation editing of genomic regions by multiplex homology-directed repair. Nature 513, 1-2.
Finiberg, E. & Ostermeier, M. PFunkel: Efficient, Expansive, User-Defined Mutagenesis. PLoS One 7, 1-10 (2012).
Wrenbeck, E. E. et al. Plasmid-based one-pot saturation mutagenesis. Nat. Methods 13, 928-930 (2016).
Wissink, E. M., Fogarty, E. A. & Grimson, A. High-throughput discovery of post-transcriptional cis-regulatory elements. BMC Genomics 17, 1-14 (2016).
Araya et al. 2016, U.S. Patent Application 20160378915A1.

What is claimed:

1. A computer implemented method for determining phenotypic impacts of molecular variants identified within a biological sample, comprising:
receiving molecular variants associated with one or more functional elements within a model system, wherein the model system comprises single-cells, cellular compartments, subcellular compartments, or synthetic compartments;
determining molecular scores or phenotype scores of the single-cells, the cellular compartments, the subcellular compartments, or the synthetic compartments;
determining molecular signals or phenotype signals associated with the molecular variants based on the respective molecular scores or phenotype scores of the single-cells, the cellular compartments, the subcellular compartments, or the synthetic compartments harboring specific molecular variants;
determining population signals associated with the molecular variants based on the molecular scores or phenotype scores of the single-cells, the cellular compartments, the subcellular compartments, or the synthetic compartments harboring specific molecular variants;
determining functional scores or functional classifications for the molecular variants based on statistical learning, wherein the statistical learning associates the molecular signals, the phenotype signals, or the population signals of molecular variants with phenotypic impacts of the molecular variants;
deriving evidence scores or evidence classifications of the molecular variants based on the functional scores or functional classifications, a modeling of the functional scores or functional classifications, a modeling of predictor scores or predictor classifications, or a modeling of hotspot scores or hotspot classifications; and
determining the phenotypic impacts of the molecular variants based on the functional scores, the functional classifications, the evidence scores, or the evidence classifications.

2. The method of claim 1, wherein the evidence scores or the evidence classifications are determined based on the molecular signals, the phenotype signals, or the population signals from the molecular variants in one or more functional elements.

3. The method of claim 1, wherein the evidence scores or evidence classifications are derived from the functional scores or functional classifications, the predictor scores or predictor classifications, or the hotspots scores or hotspot classifications.

4. The method of claim 1, wherein the evidence scores or evidence classifications are derived by applying the statistical learning using regression or classification to associate evidence scores and evidence classifications to phenotypic impacts of the molecular variants.

5. The method of claim 1, wherein the functional scores or functional classifications of the molecular variants are derived by applying statistical learning using regression or classification to associate molecular signals to phenotypic impacts of the molecular variants.

6. The method of claim 4, wherein the phenotypic impacts of the molecular variants are derived based on clinical databases, phenotype databases, population databases, molecular annotation databases, or functional databases of variants, subjects or populations.

7. The method of claim 4, wherein the phenotypic impacts of the molecular variants are derived based on molecular signals such as mutation burden, mutation rate, and mutation signatures.

8. The method of claim 1, wherein the functional scores or functional classifications of the molecular variants are derived from a plurality of statistical models generated using independent or disjoint estimates of the molecular signals, the phenotype signals, or the population signals.

9. The method of claim 1, wherein the functional scores or functional classifications of the molecular variants are derived from a Functional Modeling Engine (FME), wherein the FME is generated by applying machine learning techniques to associate non-assayed features of the molecular variants to the functional scores or functional classifications, and wherein the non-assayed features include evolutionary, population, functional, structural, dynamical, and physicochemical features.

10. The method of claim 1, wherein the predictor scores or predictor classifications of the molecular variants are derived from a Variant Interpretation Engine (VIE), wherein the VIE is generated by applying machine learning techniques to associate the functional scores or functional classifications and non-assayed features with the phenotypic impacts of the molecular variants.

11. The method of claim 1, wherein the predictor scores or predictor classifications are derived from lower-order Variant Interpretation Engines (VIEs), wherein the lower-order VIEs are functional element, functional type, or condition-specific.

12. The method of claim 1, wherein the predictor scores or predictor classifications are derived from higher-order Variant Interpretation Engines (VIEs), wherein the higher-order VIEs are pathway-, homolog family, enzyme family, or condition-specific.

13. The method of claim 1, wherein the predictor scores or predictor classifications are derived from higher-order Variant Interpretation Engines (VIEs), wherein the VIEs inform on multiple pathways-, homolog families, enzyme families, or conditions.

14. The method of claim 1, wherein the hotspot scores or hotspot classifications of the molecular variants are derived from Significantly Mutated Regions and Networks (SMRs/SMNs) computed applying spatial clustering techniques to detect regions and networks of residues with high densities of molecular variants with high or low functional scores, or specific functional classifications.

15. The method of claim 1, wherein the molecular signals comprise lower-order molecular signals of the molecular variants that are derived as summary statistics, summary statistics, descriptive statistics, inferential statistics, or Bayesian inference models of the molecular scores measured in the single-cells, the cellular compartments, the subcellular compartments, or the synthetic compartments harboring the molecular variants.

16. The method of claim 1, wherein the molecular signals comprise higher-order molecular signals of the molecular variants that are derived by applying pre-existing models that associate lower-order molecular signals to regulatory, signaling, pathway, processing, cell-cycle activities, alterations, defects, or states.

17. The method of claim 1, wherein the molecular signals comprise higher-order molecular signals of the molecular variants that are derived via unsupervised learning, feature learning, or dimensionality reduction techniques from lower-order molecular signals.

18. The method of claim 1, wherein the molecular signals comprise lower-order molecular scores corresponding to molecular measurements, molecular processes, molecular features from the single-cells, the cellular compartments, the subcellular compartments, or the synthetic compartments.

19. The method of claim 1, wherein the molecular signals comprise higher-order molecular scores of the single-cells, the cellular compartments, the subcellular compartments, or the synthetic compartments that are derived by applying pre-existing models that associate lower-order molecular scores to regulatory, signaling, pathway, processing, cell-cycle activities, alterations, defects, or states.

20. The method of claim 1, wherein the molecular signals comprise higher-order molecular scores of the single-cells, the cellular compartments, the subcellular compartments, or the synthetic compartments that are derived via unsupervised learning, feature learning, or dimensionality reduction techniques from lower-order molecular scores.

21. The method of claim 20, wherein an Autoencoder neural network is trained to learn compressed representations of lower-order molecular scores, and the Autoencoder is utilized to encode lower-order molecular signals into higher-order compressed representations.

22. The method of claim 21, wherein the Autoencoder is trained as a Denoising Autoencoder (DAE), or the Autoencoder is constructed as a neural network with fully-connected layers, or the Autoencoder is constructed as a neural network with symmetric numbers of neurons, or the Autoencoder is built with a rectified linear-units (ReLu) for activation, or the Autoencoder is trained using an Adam optimizer or the Autoencoder is celltype-, gene-, pathway-, or disorder-specific.

23. The method of claim 18, wherein the molecular measurements correspond to locus-specific measurements of gene expression, protein expression, chromatin accessibility, epigenetic modification, regulatory activity, post-transcriptional processing, post-translational modification, mutation status, mutation burden, or mutation rate of molecules within the single-cells, the cellular compartments, the subcellular compartments, or the synthetic compartments.

24. The method of claim 18, wherein the molecular processes correspond to multi-locus measurements of gene expression, protein expression, chromatin accessibility, epigenetic modification, regulatory activity, transcriptional activity, translational activity, signaling activity, pathway activity, mutation status, mutation burden, or mutation rate, among others, derived from molecular measurements within the single-cells, the cellular compartments, the subcellular compartments, or synthetic compartments.

25. The method of claim 18, wherein the molecular features correspond to global measurements of gene expression, protein expression, chromatin accessibility, epigenetic modification, regulatory activity, transcriptional activity, translational activity, signaling activity, pathway activity, mutation status, mutation burden, or mutation rate, among others, derived from molecular measurements or molecular processes within the single-cells, the cellular compartments, the subcellular compartments, or the synthetic compartments.

26. The method of claim 18, wherein the molecular measurements are derived by applying single-cell barcoding and nucleic acid sequencing techniques on populations of the single-cells, the cellular compartments, the subcellular compartments, or the synthetic compartments.

27. The method of claim 18, wherein the molecular measurements may comprise: sequencing read quality control, cellular barcode identification or quality control, molecular barcode identification or quality control, sequencing read alignment to a reference genome, sequencing read alignment filtering or quality control, mapping filtered and quality-controlled sequencing reads to functional elements, mapping filtered and quality-controlled molecular barcodes to functional elements, and mapping filtered and quality-controlled sequencing reads or molecular barcodes for specific cellular barcodes to functional elements.

28. The method of claim 1, wherein the molecular signals, the phenotype signals, or the population signals are molecular state-specific, derived from populations of the single-cells, the cellular compartments, the subcellular compartments, or the synthetic compartments from a specific molecular state to permit learning in a state-specific learning layer.

29. The method of claim 1, wherein the molecular signals, the phenotype signals, or the population signals are molecular state-agnostic, derived from populations of the single-cells, the cellular compartments, the subcellular compartments, or the synthetic compartments from a plurality of molecular states to permit learning in a state-agnostic learning layer.

30. The method of claim 1, wherein the molecular signals, the phenotype signals, or the population signals are molecular state-ordered, derived from populations of the single-cells, the cellular compartments, the subcellular compartments, or the synthetic compartments from a plurality of molecular states to permit learning in a multi-state learning layer.

31. The method of claim 1, wherein molecular states of the single-cells, the cellular compartments, the subcellular compartments, or the synthetic compartments are derived by applying pre-existing models associating molecular scores or phenotype scores to the molecular states, wherein the models assign single-cells to phases of cell-cycle based on previously characterized gene-expression signatures.

32. The method of claim 1, wherein molecular states of the single-cells, the cellular compartments, the subcellular compartments, or the synthetic compartments are derived via unsupervised learning, feature learning, or dimensionality reduction techniques of molecular scores or phenotype scores across the single-cells, the cellular compartments, the subcellular compartments, or the synthetic compartments.

33. The method of claim 1, wherein the molecular signals, the phenotype signals, or the population signals are computed from independent or disjoint populations of single-cells, cellular compartments, subcellular compartments, or synthetic compartments selected from the single-cells, the cellular compartments, the subcellular compartments, or they synthetic compartments harboring a same molecular variant via random sampling.

34. The method of claim 1, wherein the molecular variants correspond to coding or non-coding variants within functional elements, genes and pathways associated with Mendelian disorders.

35. The method of claim 1, wherein the molecular variants correspond to coding or non-coding variants within functional elements, genes and pathways associated with known cancer-drivers.

36. The method of claim 1, wherein the molecular variants correspond to coding or non-coding variants within functional elements, genes and pathways associated with variation in drug response.

37. The method of claim 1, wherein the molecular variants correspond to coding or non-coding variants within previously identified mutational hotspots of functional elements, genes and pathways associated with other clinically-valuable genes.

38. The method of claim 1, wherein the molecular variants correspond to coding or non-coding variants within previously identified mutational hotspots of functional elements, genes and pathways associated with Mendelian disorders.

39. The method of claim 1, wherein the molecular variants correspond to coding or non-coding variants within previously identified mutational hotspots of functional elements, genes and pathways associated with known cancer-drivers.

40. The method of claim 1, wherein the molecular variants correspond to coding or non-coding variants within previously identified mutational hotspots of functional elements, genes and pathways associated with variation in drug response.

41. The method of claim 1, wherein the molecular variants correspond to coding or non-coding variants within previously identified mutational hotspots of functional elements, genes and pathways associated with other clinically-valuable genes.

42. The method of claim 1, wherein the molecular variants correspond to coding or non-coding variants within 10 bp of previously identified mutational hotspots of functional elements, genes and pathways associated with Mendelian disorders.

43. The method of claim 1, wherein the molecular variants correspond to coding or non-coding variants within 10 bp of previously identified mutational hotspots of functional elements, genes and pathways associated with known cancer-drivers.

44. The method of claim 1, wherein the molecular variants correspond to coding or non-coding variants within 10 bp of previously identified mutational hotspots of functional elements, genes and pathways associated with variation in drug response.

45. The method of claim 1, wherein the molecular variants correspond to coding or non-coding variants within 10 bp of previously identified mutational hotspots of functional elements, genes and pathways associated with other clinically-valuable genes.

46. The method of claim 1, wherein the molecular variants correspond to coding or non-coding variants within 50 bp of previously identified mutational hotspots of functional elements, genes and pathways associated with Mendelian disorders.

47. The method of claim 1, wherein the molecular variants correspond to coding or non-coding variants within 50 bp of previously identified mutational hotspots of functional elements, genes and pathways associated with known cancer-drivers.

48. The method of claim 1, wherein the molecular variants correspond to coding or non-coding variants within 50 bp of previously identified mutational hotspots of functional elements, genes and pathways associated with variation in drug response.

49. The method of claim 1, wherein the molecular variants correspond to coding or non-coding variants within 50 bp of previously identified mutational hotspots of functional elements, genes and pathways associated with other clinically-valuable genes.

50. The method of claim 1, wherein the molecular variants correspond to coding or non-coding variants within 100 bp of previously identified mutational hotspots of functional elements, genes and pathways associated with Mendelian disorders.

51. The method of claim 1, wherein the molecular variants correspond to coding or non-coding variants within 100 bp of previously identified mutational hotspots of functional elements, genes and pathways associated with known cancer-drivers.

52. The method of claim 1, wherein the molecular variants correspond to coding or non-coding variants within 100 bp of previously identified mutational hotspots of functional elements, genes and pathways associated with variation in drug response.

53. The method of claim 1, wherein the molecular variants correspond to coding or non-coding variants within 100 bp of previously identified mutational hotspots of functional elements, genes and pathways associated with other clinically-valuable genes.

54. The method of claim 1, wherein the molecular variants correspond to coding or non-coding variants within 500 bp of previously identified mutational hotspots of functional elements, genes and pathways associated with Mendelian disorders.

55. The method of claim 1, wherein the molecular variants correspond to coding or non-coding variants within 500 bp of previously identified mutational hotspots of functional elements, genes and pathways associated with known cancer-drivers.

56. The method of claim 1, wherein the molecular variants correspond to coding or non-coding variants within 500 bp of previously identified mutational hotspots of functional elements, genes and pathways associated with variation in drug response.

57. The method of claim 1, wherein the molecular variants correspond to coding or non-coding variants within 500 bp of previously identified mutational hotspots of functional elements, genes and pathways associated with other clinically-valuable genes.

58. The method of claim 1, wherein the molecular variants correspond to coding or non-coding variants within 1,000 bp of previously identified mutational hotspots of functional elements, genes and pathways associated with Mendelian disorders.

59. The method of claim 1, wherein the molecular variants correspond to coding or non-coding variants within 1,000 bp of previously identified mutational hotspots of functional elements, genes and pathways associated with known cancer-drivers.

60. The method of claim 1, wherein the molecular variants correspond to coding or non-coding variants within 1,000 bp of previously identified mutational hotspots of functional elements, genes and pathways associated with variation in drug response.

61. The method of claim 1, wherein the molecular variants correspond to coding or non-coding variants within 1,000 bp of previously identified mutational hotspots of functional elements, genes and pathways associated with other clinically-valuable genes.

62. The method of claim 1, wherein the molecular variants correspond to coding or non-coding variants within previously identified constrained regions of functional elements, genes and pathways associated with Mendelian disorders.

63. The method of claim 1, wherein the molecular variants correspond to coding or non-coding variants within previously identified constrained regions of functional elements, genes and pathways associated with known cancer-drivers.

64. The method of claim 1, wherein the molecular variants correspond to coding or non-coding variants within previously identified constrained regions of functional elements, genes and pathways associated with variation in drug response.

65. The method of claim 1, wherein the molecular variants correspond to coding or non-coding variants within previously identified constrained regions of functional elements, genes and pathways associated with other clinically-valuable genes.

66. The method of claim 1, wherein the molecular variants correspond to coding or non-coding variants within 10 bp of previously identified constrained regions of functional elements, genes and pathways associated with Mendelian disorders.

67. The method of claim 1, wherein the molecular variants correspond to coding or non-coding variants within 10 bp of previously identified constrained regions of functional elements, genes and pathways associated with known cancer-drivers.

68. The method of claim 1, wherein the molecular variants correspond to coding or non-coding variants within 10 bp of previously identified constrained regions of functional elements, genes and pathways associated with variation in drug response.

69. The method of claim 1, wherein the molecular variants correspond to coding or non-coding variants within 10 bp of previously identified constrained regions of functional elements, genes and pathways associated with other clinically-valuable genes.

70. The method of claim 1, wherein the molecular variants correspond to coding or non-coding variants within 50 bp of previously identified constrained regions of functional elements, genes and pathways associated with Mendelian disorders.

71. The method of claim 1, wherein the molecular variants correspond to coding or non-coding variants within 50 bp of previously identified constrained regions of functional elements, genes and pathways associated with known cancer-drivers.

72. The method of claim 1, wherein the molecular variants correspond to coding or non-coding variants within 50 bp of previously identified constrained regions of functional elements, genes and pathways associated with variation in drug response.

73. The method of claim 1, wherein the molecular variants correspond to coding or non-coding variants within 50 bp of previously identified constrained regions of functional elements, genes and pathways associated with other clinically-valuable genes.

74. The method of claim 1, wherein the molecular variants correspond to coding or non-coding variants within 100 bp of previously identified constrained regions of functional elements, genes and pathways associated with Mendelian disorders.

75. The method of claim 1, wherein the molecular variants correspond to coding or non-coding variants within 100 bp of previously identified constrained regions of functional elements, genes and pathways associated with known cancer-drivers.

76. The method of claim 1, wherein the molecular variants correspond to coding or non-coding variants within 100 bp of previously identified constrained regions of functional elements, genes and pathways associated with variation in drug response.

77. The method of claim 1, wherein the molecular variants correspond to coding or non-coding variants within 100 bp of previously identified constrained regions of functional elements, genes and pathways associated with other clinically-valuable genes.

78. The method of claim 1, wherein the molecular variants correspond to coding or non-coding variants within 500 bp of previously identified constrained regions of functional elements, genes and pathways associated with Mendelian disorders.

79. The method of claim 1, wherein the molecular variants correspond to coding or non-coding variants within 500 bp of previously identified constrained regions of functional elements, genes and pathways associated with known cancer-drivers.

80. The method of claim 1, wherein the molecular variants correspond to coding or non-coding variants within 500 bp of previously identified constrained regions of functional elements, genes and pathways associated with variation in drug response.

81. The method of claim 1, wherein the molecular variants correspond to coding or non-coding variants within 500 bp of previously identified constrained regions of functional elements, genes and pathways associated with other clinically-valuable genes.

82. The method of claim 1, wherein the molecular variants correspond to coding or non-coding variants within 1,000 bp of previously identified constrained regions of functional elements, genes and pathways associated with Mendelian disorders.

83. The method of claim 1, wherein the molecular variants correspond to coding or non-coding variants within 1,000 bp of previously identified constrained regions of functional elements, genes and pathways associated with known cancer-driver.

84. The method of claim 1, wherein the molecular variants correspond to coding or non-coding variants within 1,000 bp of previously identified constrained regions of functional elements, genes and pathways associated with variation in drug response.

85. The method of claim 1, wherein the molecular variants correspond to coding or non-coding variants within 1,000 bp of previously identified constrained regions of functional elements, genes and pathways associated with other clinically-valuable genes.

86. The method of claim 1, wherein the phenotype scores of the single-cells, the cellular compartments, the subcellular compartments, or the synthetic compartments represent phenotypic associations of the molecular variants identified within the single-cells, the cellular compartments, the subcellular compartments, or the synthetic compartments.

87. The method of claim 1, wherein the phenotype scores of the single-cells, the cellular compartments, the subcellular compartments, or the synthetic compartments comprise lower-order phenotype scores, wherein the lower-order phenotype scores correspond to scores or classifications generated by a phenotype model through the use of statistical learning techniques that associate molecular scores and molecular states of model systems with the phenotypic impacts of molecular variants within each model system.

88. The method of claim 87, wherein the phenotype model is generated using a neural network architecture for single-task or multi-task statistical learning that associates molecular scores from one or more functional elements with one or more phenotypic impacts of molecular variants in the one or more functional elements.

89. The method of claim 1, wherein the phenotype scores of the single-cells, the cellular compartments, the subcellular compartments, or the synthetic compartments comprise higher-order phenotype scores, wherein the higher-order phenotype scores are derived by applying pre-existing models that associate lower-order phenotype scores to regulatory, signaling, pathway, processing, cell-cycle activities, alterations, defects, or states.

90. The method of claim 1, wherein the phenotype scores of the single-cells, the cellular compartments, the subcellular compartments, or the synthetic compartments comprise higher-order phenotype scores, wherein the higher-order phenotype scores are derived via unsupervised learning, feature learning, or dimensionality reduction techniques from lower-order phenotype scores.

91. The method of claim 1, wherein the phenotype signals associated with the molecular variants comprise lower-order phenotype signals associated with the molecular variants, wherein the lower-order phenotype signals associated with the molecular variants are derived as summary statistics, descriptive statistics, inferential statistics, Bayesian inference models of the phenotype scores measured in the single-cells, the cellular compartments, the subcellular compartments, or the synthetic compartments harboring the molecular variants.

92. The method of claim 1, wherein the phenotype signals associated with the molecular variants comprise higher-order phenotype signals associated with the molecular variants, wherein the higher-order phenotype signals associated with the molecular variants are derived by applying pre-existing models that associate lower-order phenotype signals to regulatory, signaling, pathway, processing, cell-cycle activities, alterations, defects, or states.

93. The method of claim 1, wherein the phenotype signals associated with the molecular variants comprises higher-order phenotype signals associated with the molecular variants, wherein the higher-order phenotype signals associated with the molecular variants are derived via unsupervised learning, feature learning, or dimensionality reduction techniques from lower-order phenotype signals.

94. The method of claim 1, further comprising:
accessing a collection of molecular variants with putative or known phenotypic impacts from pre-existing sources;
increasing the collection of molecular variants with putative or known phenotypic impacts using a prediction model;
selecting a first set of genotypes with putative or known phenotypic impacts using a sampling model;
selecting a second set of genotypes with unknown, putative, or known phenotypic impacts using a sampling model;
selecting a third set of genotypes with unknown, putative, or known phenotypic impacts using a sampling model;
generating a functional model by applying statistical learning techniques that associates molecular signals, phenotype signals, or population signals of the first set of genotypes with putative or known phenotypic impacts;
generating predicted phenotypic impacts for the second set of genotypes by applying the functional model to make predictions based on molecular signals, phenotype signals, or population signals of the second set of genotypes;
generating an inference model by applying statistical learning techniques, wherein the inference model associates non-assayed features with phenotypic impacts of molecular variants; and
generating predicted phenotypic impacts of the third set of genotypes by applying the inference model to make predictions based on non-assayed features of the third set of genotypes.

95. The method of claim 94, wherein the prediction model is gene-specific, domain-specific, homolog-specific, or a genome-wide computational predictor or functional assay.

96. The method of claim 94, wherein the prediction model provides performance or confidence estimates for each prediction of the prediction model.

97. The method of claim 94, wherein a positive predictive value (PPV) of the prediction model comprises a function of a performance or confidence estimate of a prediction of the prediction model.

98. The method of claim 94, wherein a negative predictive value (NPV) of the prediction model comprises a function of a performance or confidence estimate of a prediction of the prediction model.

99. The method of claim 94, wherein the prediction model is a molecular impact predictor.

100. The method of claim 94, wherein the prediction model predicts early termination, non-sense, or truncating molecular variants in protein-coding functional elements are loss-of-function variants.

101. The method of claim 94, wherein the prediction model predicts synonymous or silent molecular variants in protein-coding functional elements are neutral variants.

102. The method of claim 1, further comprising:
generating a functional model by applying statistical learning techniques that combine the molecular signals, the phenotype signals, or the population signals and the phenotypic impacts of the molecular variants of the functional elements.

103. The method of claim 102, wherein the generating the functional model further comprises:
generating the functional model using a neural network architecture for single-task or multi-task learning that associates the molecular signals, the phenotype signals, or the population signals from the functional elements with the one or more phenotypic impacts of the molecular variants of the functional elements.

104. The method of claim 1, further comprising:
generating a phenotype model by applying statistical learning techniques that combine the molecular scores and the phenotypic impacts of the molecular variants of the functional elements.

105. The method of claim 104, wherein the generating the phenotype model further comprises:
generating a phenotype model using a neural network architecture for single-task or multi-task learning that associates the molecular scores from the functional elements with the one or more phenotypic impacts of the molecular variants of the functional elements.

106. The method of claim 1, further comprising:
introducing the molecular variants into the functional elements within the single-cells, the cellular compartments, the subcellular compartments, or the synthetic compartments;
identifying the molecular variants within the single-cells, the cellular compartments, the subcellular compartments, or the synthetic compartments;
determining the phenotypic impacts of the molecular variants within the single-cells, the cellular compartments, the subcellular compartments, or the synthetic compartments; and
determining molecular measurements, molecular features, or molecular processes within the single-cells, the cellular compartments, the subcellular compartments, or the synthetic compartments.

107. The method of claim 1, wherein the population signals associated with the molecular variants describe a distribution of the single-cells, the cellular compartments, the subcellular compartments, or the synthetic compartments associated with the molecular variants across subpopulations of single-cells, cellular compartments, subcellular compartments, or synthetic compartments from distinct molecular states.

108. The method of claim 1, wherein the population signals associated with molecular variants describe dynamics of the single-cells, the cellular compartments, the subcellular compartments, or the synthetic compartments associated with the molecular variants across subpopulations of the single-cells, the cellular compartments, the subcellular compartments, or the synthetic compartments from distinct molecular states.

109. The method of claim 1, wherein the population signals associated with the molecular variants describe changes to a distribution of the single-cells, the cellular compartments, the subcellular compartments, or the synthetic compartments across subpopulations of the single-cells, the cellular compartments, the subcellular compartments, or the synthetic compartments from distinct molecular states that are associated with the molecular variants.

110. The method of claim 1, wherein the population signals associated with the molecular variants describe changes to dynamics of the single-cells, the cellular compartments, the subcellular compartments, or the synthetic compartments across subpopulations of the single-cells, the cellular compartments, the subcellular compartments, or the synthetic compartments from distinct molecular states that are associated with the molecular variants.

111. The methods of claim 107, wherein clustering techniques are applied to cluster and assign the single-cells, the cellular compartments, the subcellular compartments, or the synthetic compartments based on the molecular scores or the phenotype scores.

112. The method of claim 111, wherein Gaussian Mixture Models (GMMs) are applied to cluster and assign the single-cells, the cellular compartments, the subcellular compartments, or the synthetic compartments to a defined number of molecular states.

113. The method of claim 111, wherein Variational Gaussian Mixture Models (VGMMs) are applied to cluster and assign the single-cells, the cellular compartments, the subcellular compartments, or the synthetic compartments to an inferred number of molecular states using Dirichlet processes.

114. The method of claim 107, wherein the population signals associated with the molecular variants are determined as a fraction of the single-cells, the cellular compartments, the subcellular compartments, or the synthetic compartments associated with the molecular variants corresponding to specific molecular states.

115. The method of claim 1, wherein the molecular scores or the phenotype scores of the molecular variants comprise adjusted molecular scores or phenotype scores computed as a difference between the molecular scores or the phenotype scores of the molecular variants and the molecular scores or the phenotype scores of reference molecular variants or reference single-cells, cellular compartments, subcellular compartments, or synthetic compartments.

116. The method of claim 1, wherein the molecular scores or the phenotype scores of the molecular variants comprise adjusted molecular scores or phenotype scores computed by normalizing the molecular scores or the phenotype scores of the molecular variants against molecular scores or phenotype scores of reference molecular variants or reference single-cells, cellular compartments, subcellular compartments, or synthetic compartments.

117. The method of claim 1, wherein molecular signals, phenotype signals, or population signals of molecular variants comprise adjusted molecular signals, phenotype signals, or population signals, respectively, computed as the difference between the molecular signals, phenotype signals, or population signals of molecular variants and the molecular signals, phenotype signals, or population signals of reference molecular variants.

118. The method of claim 1, wherein the molecular signals, the phenotype signals, or the population signals associated with the molecular variants comprise adjusted molecular signals, phenotype signals, or population signals, respectively, computed by normalizing the molecular signals, the phenotype signals, or the population signals associated with the molecular variants by molecular signals, phenotype signals, or population signals of reference molecular variants.

119. The method of claim 1, wherein the molecular signals, the phenotype signals, or the population signals associated with the molecular variants comprise adjusted molecular signals, phenotype signals, or population signals, respectively, computed as quantiles of the molecular signals, the phenotype signals, or the population signals associated with the molecular variants among molecular signals, phenotype signals, or population signals of reference molecular variants.

120. A computer implemented method, further comprising:
selecting a first set of genotypes with phenotypic impacts;
selecting a second set of genotypes with phenotypic impacts;
applying single-cell capture or barcoding techniques to obtain molecules from a first cell number of single-cells, cellular compartments, subcellular compartments, or synthetic compartments associated with the first set of genotypes;
obtaining a first read number of molecular reads per model system by performing sequencing, sequencing read quality control, cellular barcode identification or quality control, molecular barcode identification or quality control, sequencing read alignment to a reference genome, or read alignment filtering or quality control using a model system associated with the first set of genotypes;
applying single-cell capture or barcoding techniques to obtain molecules from a second cell number of the single-cells, the cellular compartments, the subcellular compartments, or the synthetic compartments associated with the first set of genotypes;
obtaining a second read number of molecular reads per model by performing sequencing, sequencing read quality control, cellular barcode identification or quality control, molecular barcode identification or quality control, sequencing read alignment to a reference genome, or read alignment filtering or quality control using the model system associated with the first set of genotypes;
deriving total molecular reads or total molecular measurements from a total read number of molecular reads per model system from a total cell number of single-cells, cellular compartments, subcellular compartments, or synthetic compartments per genotype;
generating a total dimensionality reduction model by applying statistical learning techniques for feature selection or dimensionality reduction to determine molecular scores, phenotype scores, molecular signals, phenotype signals, or population signals for the first set of genotypes utilizing the total molecular reads and the total molecular measurements;
generating a total functional model by applying statistical learning techniques that associate molecular signals, phenotype signals, or population signals from the total dimensionality reduction model with phenotypic impacts for the first set of genotypes utilizing the total molecular reads and the total molecular measurements;
determining a threshold performance of functional scores or functional classifications using the total cell number, the total read number, the total dimensionality reduction model, or the total functional model for prediction of the phenotypic impacts of the first set of genotypes;
deriving optimal molecular reads or optimal molecular measurements from an optimal read number of molecular reads per model system from an optimal cell number of single-cells, cellular compartments, subcellular compartments, or synthetic compartments per genotype, where the optimal molecular reads and the optimal molecular measurements are obtained by subsampling the total molecular reads or the total molecular measurements;
generating an optimal dimensionality reduction model by applying statistical learning techniques for feature selection or dimensionality reduction to determine molecular scores, phenotype scores, molecular signals, phenotype signals, or population signals for the first set of genotypes using the optimal molecular reads and the optimal molecular measurements;
generating an optimal functional model by applying statistical learning techniques that associate molecular signals, phenotype signals, or population signals from the optimal dimensionality reduction model with phenotypic impacts for the first set of genotypes using the optimal molecular reads and the optimal molecular measurements;
validating the threshold performance of the functional scores or functional classifications based on the optimal cell number, the optimal read number, the optimal dimensionality reduction model, or the optimal functional model for prediction of the phenotypic impacts of the first set of genotypes;
applying single-cell capture or barcoding techniques to obtain molecules from the optimal cell number of single-cells, cellular compartments, subcellular compartments, or synthetic compartments associated with the second set of genotypes;
obtaining the optimal read number of molecular reads per model system by performing sequencing, sequencing read quality control, cellular barcode identification or quality control, molecular barcode identification or quality control, sequencing read alignment to a reference genome, or read alignment filtering or quality control using a model system associated with the second set of genotypes; and
generating functional scores or functional classifications for the second set of genotypes based on the optimal cell number, the optimal read number, the optimal dimensionality reduction model, or the optimal functional model.

121. A computer implemented method for scoring phenotypic impacts of molecular variants, comprising:
evaluating an evidence dataset based on an accuracy of the evidence dataset;
validating the evidence dataset based on the accuracy of the evidence dataset;
optimizing the evidence dataset based on the accuracy of the evidence dataset; and
determining the phenotypic impacts of the molecular variants based on the evaluating, validating, and optimizing of the evidence dataset.

122. The method of claim 121, wherein the evidence dataset comprises functional scores or functional classifications of molecular variants based on machine learning models associating molecular signals, phenotype signals, or population signals of the molecular variants with the phenotypic impacts of the molecular variants.

123. The method of claim 121, wherein the evidence dataset comprises predictor scores or predictor classifications from genome-wide, homolog-specific, enzyme class-specific, domain-specific, or gene-specific computational predictors.

124. The method of claim 121, wherein the evidence dataset comprises hotspot scores or hotspot classifications from mutational hotspots.

125. The method of claim 121, wherein the evidence datasets comprises population scores or population classifications from variant classifications derived on a basis of population genomics metrics.

126. The method of claim 121, further comprising:
computing evaluation metrics to assess concordance between the evidence dataset and functional scores or functional classifications.

127. The method of claim 121, wherein the evaluation metrics comprise a Pearson's correlation coefficient, a Spearman's rank-order correlation, a Kendall correlation, a Matthew's correlation coefficient, a Cohen's kappa coefficient, a Youden's index, a F-measure, a true positive rate, a true negative rate, a positive predictive value, a negative predictive value, a positive likelihood ratio, a negative likelihood ratio, or a diagnostic odds ratio.

128. The method of claim 121, wherein the validating of the evidence dataset comprises validating the evidence dataset based on the evaluation metrics.

129. The method of claim 121, wherein the optimizing of the evidence dataset comprises selecting or removing data within the evidence dataset based on the evaluation metrics.

130. A computer implemented method for scoring phenotypic impacts of molecular variants, comprising;
evaluating an evidence dataset based on an inherent bias of the evidence dataset;
validating the evidence dataset based on the inherent bias of the evidence dataset;
optimizing the evidence dataset based on the inherent bias of the evidence dataset; and
determining scores of the phenotypic impacts of the molecular variants based on the evaluating, validating, and optimizing evidence dataset.

131. The method of claim 130, wherein a bias of the evidence dataset is measured as a statistical distance between an observed evidence score or evidence classification of variants in the evidence dataset against expected evidence scores or evidence classifications of variants in a reference dataset.

132. The method of claim 130, wherein an ascertainment bias of the evidence dataset is measured as a statistical distance between observed features and properties of variants in the evidence dataset against expected features and properties of variants in a reference dataset defined on a basis of a matching quantiles or classifications.

133. The method of claim 130, wherein an ascertainment bias of the evidence dataset is measured as a statistical distance between observed features and properties of the variants in the evidence dataset against expected features and properties of variants in a reference dataset defined on a basis of a matching distribution of evidence scores or evidence classifications.

134. The method of claim 130, wherein the validating of the evidence dataset comprises validating the evidence dataset based on a target evaluation bias metric.

135. The method of claim 130, wherein the optimizing of the evidence dataset comprises selecting or removing data within the evidence dataset based on target validation criteria.

136. A system, comprising:
a memory; and
at least one processor coupled to the memory and configured to:
receive molecular variants associated with one or more functional elements within a model system, wherein the model system comprises single-cells, cellular compartments, subcellular compartments, or synthetic compartments;
determine molecular scores or phenotype scores of the single-cells, the cellular compartments, the subcellular compartments, or the synthetic compartments;
determine molecular signals or phenotype signals associated with the molecular variants based on the respective molecular scores or phenotype scores of the single-cells, the cellular compartments, the subcellular compartments, or the synthetic compartments harboring specific molecular variants;
determine population signals associated with the molecular variants based on the molecular scores or phenotype scores of the single-cells, the cellular compartments, the subcellular compartments, or the synthetic compartments harboring specific molecular variants;
determine functional scores or functional classifications for the molecular variants based on statistical learning, wherein the statistical learning associates the molecular signals, the phenotype signals, or the population signals of molecular variants with phenotypic impacts of the molecular variants;
derive evidence scores or evidence classifications of the molecular variants based on the functional scores or functional classifications, a modeling of the functional scores or functional classifications, a modeling of predictor scores or predictor classifications, or a modeling of hotspot scores or hotspot classifications; and
determine the phenotypic impacts of the molecular variants based on the functional scores, the functional classifications, the evidence scores, or the evidence classifications.

137. A tangible computer-readable device having instructions stored thereon that, when executed by at least one computing device, causes the at least one computing device to perform operations comprising:
receive molecular variants associated with one or more functional elements within a model system, wherein the model system comprises single-cells, cellular compartments, subcellular compartments, or synthetic compartments;
determining molecular scores or phenotype scores of the single-cells, the cellular compartments, the subcellular compartments, or the synthetic compartments;
determining molecular signals or phenotype signals associated with the molecular variants based on the respective molecular scores or phenotype scores of the single-cells, the cellular compartments, the subcellular compartments, or the synthetic compartments harboring specific molecular variants;
determining population signals associated with the molecular variants based on the molecular scores or phenotype scores of the single-cells, the cellular compartments, the subcellular compartments, or the synthetic compartments harboring specific molecular variants;
determining functional scores or functional classifications for the molecular variants based on statistical learning, wherein the statistical learning associates the molecular signals, the phenotype signals, or the population signals of molecular variants with phenotypic impacts of the molecular variants;
deriving evidence scores or evidence classifications of the molecular variants based on the functional scores or functional classifications, a modeling of the functional scores or functional classifications, a modeling of predictor scores or predictor classifications, or a modeling of hotspot scores or hotspot classifications; and
determining the phenotypic impacts of the molecular variants based on the functional scores, the functional classifications, the evidence scores, or the evidence classifications.
